(19) United States
(12) Patent Application Publication
(10) Pub. No.: US 2024/0108618 A1
(43) Pub. Date: Apr. 4, 2024

(54) KRAS G12C INHIBITOR DOSING REGIMENS

(71) Applicant: Eli Lilly and Company, Indianapolis, IN (US)

(72) Inventors: David Michael HYMAN, Westport, CT (US); Xueqian GONG, Carmel, IN (US); Sheng-Bin PENG, Carmel, IN (US); Chong SI, Zionsville, IN (US); Melinda Dale WILLARD, Zionsville, IN (US)

(21) Appl. No.: 18/344,134

(22) Filed: Jun. 29, 2023

Related U.S. Application Data
(60) Provisional application No. 63/496,447, filed on Apr. 17, 2023.
(60) Provisional application No. 63/486,785, filed on Feb. 24, 2023.
(60) Provisional application No. 63/357,227, filed on Jun. 30, 2022.

(51) Int. Cl. A61K 31/4985; A61K 9/48; A61K 31/519; A61K 31/555; A61K 33/243; A61P 35/00

(52) U.S. Cl. CPC A61K 31/4985; A61K 9/48; A61K 31/519; A61K 31/555; A61K 33/243; A61P 35/00; A61K 2039/505

(57) ABSTRACT

Disclosed herein are dosing regimens for the administration of a compound of Formula I:

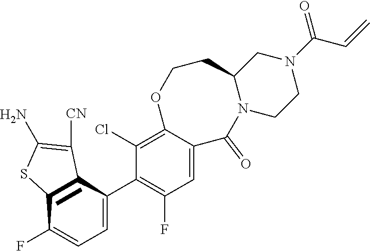

or pharmaceutically acceptable salt thereof, or in combination with one or more of a second therapeutic agent, to a patient in need of such treatment.

### BACKGROUND

Oncogenic KRas mutations have been identified in approximately 30% of human cancers and have been demonstrated to activate multiple downstream signaling pathways. Despite the prevalence of KRas mutations, it has been a difficult therapeutic target.

WO 2021/118877 and US 2021/0179633 A1 each disclose compounds or salts thereof that can be used as KRas G12C inhibitors. An example of a KRAS G12C inhibitor of interest is 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, which has the following structure:

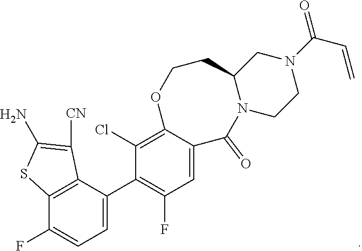 [SMILES: C=CC(=O)N1CCN2C(=O)c3cc(F)c(-c4ccc(F)c5sc(N)c(C#N)c45)c(Cl)c3OCC[C@H]2C1]

This compound was disclosed as Example 35 in each of WO 2021/118877 and US 2021/0179633 A1. As disclosed therein, this compound exists as atropisomers. Further as discussed in Preparations 167 and 168 of these references, the atropisomers may be separated using silica gel flash column chromatography. Preparation 167 further teaches the desired diastereomer is the second diastereomer to elute off the silica gel flash column, when eluting with 0-30% acetone/hexanes.

This second diastereomer corresponds to the M atropisomer. The name of this compound is 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M Atropisomer (hereinafter “Formula”) having the following structure:

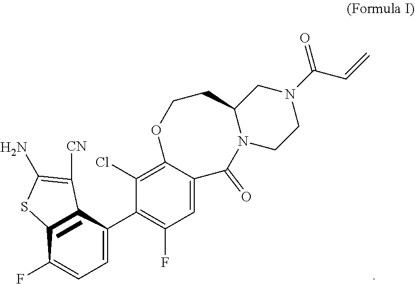

The compound of Formula I is currently undergoing clinical testing (ClinicalTrials.gov Identifier: NCT04956640) to assess its utility in treating patients having cancer that is treatable by inhibiting KRAS G12C.

It would be useful to develop new treatment regimens and protocols that use the compound of Formula I, either as a monotherapy, in combination with one or more other therapeutic agents, or as part of neoadjuvant, adjuvant, advanced, or metastatic therapy, to treat cancer. It would be useful to develop more tolerable treatment regimens and protocols than current treatment regimens or protocols. It would be useful to develop less toxic treatment regimens and protocols than current treatment regimens or protocols.

### SUMMARY

Disclosed herein are methods and uses of the compound of Formula I, or pharmaceutically acceptable salts thereof, to treat cancers that are treatable by inhibiting KRAS G12C.

In an aspect, disclosed herein is a method of treating a KRAS G12C mutant cancer comprising administering to a patient in need of such treatment, a dose between about 50 mg and about 200 m of compound of Formula I:

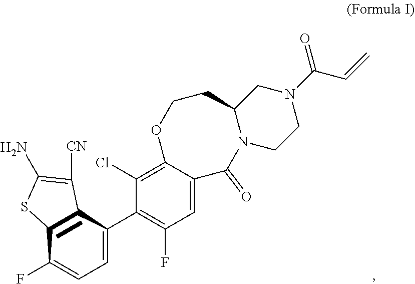

or a pharmaceutically acceptable salt thereof.

In another aspect, disclosed herein is a method of treating a KRAS G12C mutant cancer comprising administering to a patient in need of such treatment, a dose of about 150 mg of the compound of Formula I, or a pharmaceutically acceptable salt thereof.

In another aspect, disclosed herein is a method of treating a KRAS G12C mutant cancer comprising administering to a patient in need of such treatment, a dose between about 50 mg and about 200 mg of the compound of Formula I, or a pharmaceutically acceptable salt thereof.

In another aspect, disclosed herein is a method of treating a KRAS G12C mutant cancer comprising administering to a patient in need of such treatment, a dose of about 150 mg of the compound of Formula I, or a pharmaceutically acceptable salt thereof.

In still another aspect, disclosed herein is a method of treating a KRAS G12C mutant cancer comprising administering to a patient in need of such treatment, a dose between about 50 mg and about 200 mg of the compound of Formula I, or a pharmaceutically acceptable salt thereof, wherein the KRAS G12C mutant cancer is KRAS G12C-mutant advanced NSCLC.

In still another aspect, disclosed herein is a method of treating a KRAS G12C mutant cancer comprising administering to a patient in need of such treatment, a dose of about 150 mg of the compound of Formula I, or a pharmaceutically acceptable salt thereof, wherein the KRAS G12C mutant cancer is KRAS G12C-mutant advanced NSCLC.

In still another aspect, disclosed herein is a method of treating a KRAS G12C mutant cancer comprising administering to a patient in need of such treatment, a dose between about 50 mg and about 200 mg of the compound of Formula I, or a pharmaceutically acceptable salt thereof, wherein the KRAS G12C mutant cancer is KRAS G12C-mutant advanced CRC.

In still another aspect, disclosed herein is a method of treating a KRAS G12C mutant cancer comprising administering to a patient in need of such treatment, a dose of about 150 mg of the compound of Formula I, or a pharmaceutically acceptable salt thereof, wherein the KRAS G12C mutant cancer is KRAS G12C-mutant advanced CRC.

In still another aspect, disclosed herein is a method of treating a KRAS G12C mutant cancer comprising administering to a patient in need of such treatment, a dose between about 50 mg and about 200 mg of the compound of Formula I, or a pharmaceutically acceptable salt thereof, wherein the KRAS G12C mutant cancer is KRAS G12C-mutant pancreatic cancer.

In still another aspect, disclosed herein is a method of treating a KRAS G12C mutant cancer comprising administering to a patient in need of such treatment, a dose of about 150 mg of the compound of Formula I, or a pharmaceutically acceptable salt thereof, wherein the KRAS G12C mutant cancer is KRAS G12C-mutant pancreatic cancer.

In still another aspect, disclosed herein is a method of treating a KRAS G12C mutant cancer comprising administering to a patient in need of such treatment, a dose of about 100 mg of the compound of Formula I, or a pharmaceutically acceptable salt thereof, wherein the KRAS G12C mutant cancer is KRAS G12C-mutant pancreatic cancer.

In still another aspect, disclosed herein is a method of treating a KRAS G12C mutant cancer comprising administering to a patient in need of such treatment, a dose of about 50 mg of the compound of Formula I, or a pharmaceutically acceptable salt thereof, wherein the KRAS G12C mutant cancer is KRAS G12C-mutant pancreatic cancer.

In yet another aspect, disclosed herein is a method of treating a KRAS G12C mutant cancer, comprising
administering to a patient in need of such treatment, a first dose between about 50 mg and about 200 mg of the compound of Formula I, or a pharmaceutically acceptable salt thereof, or a pharmaceutically acceptable salt thereof;
monitoring the patient for a dose limiting toxicity (DLT); and
administering a second dose of the compound of Formula I, or a pharmaceutically acceptable salt thereof, if the patient exhibits the DLT, wherein the second dose is reduced as compared to the first dose.

In yet another aspect, disclosed herein is a method of treating a KRAS G12C mutant cancer, comprising
administering to a patient in need of such treatment, a first dose of about 150 mg of the compound of Formula I, or a pharmaceutically acceptable salt thereof;
monitoring the patient for a DLT; and
administering a second dose of the compound of Formula I, or a pharmaceutically acceptable salt thereof, if the patient exhibits the DLT, wherein the second dose is reduced as compared to the first dose.

In another aspect, disclosed herein is a method of treating a KRAS G12C mutant cancer comprising administering to a patient in need thereof, a dose between about 50 mg and about 200 mg of the compound of Formula I, or a pharmaceutically acceptable salt thereof, in simultaneous, separate or sequential combination with one or more of a second therapeutic agent. In an embodiment the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

In another aspect, disclosed herein is a method of treating a KRAS G12C mutant cancer comprising administering to a patient in need thereof, a dose of about 150 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer comprising administering to a patient in need thereof, a dose of about 100 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer comprising administering to a patient in need thereof, a dose of about 50 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a compound of Formula I:

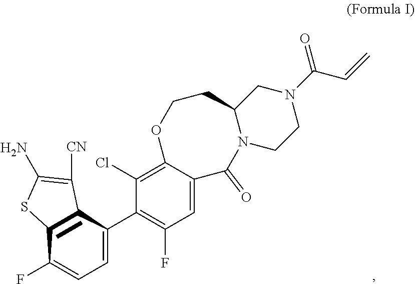

or a pharmaceutically acceptable salt thereof, for use in treatment of a KRAS G12C mutant cancer, wherein the compound of Formula I, or a pharmaceutically acceptable salt thereof, is administered at a dose between about 50 mg and about 200 mg.

In another aspect, disclosed herein is the compound of Formula I, or a pharmaceutically acceptable salt thereof, for use in treatment of a KRAS G12C mutant cancer, wherein the compound, is administered at a dose of about 150 mg.

In another aspect, disclosed herein is the compound of Formula I, or a pharmaceutically acceptable salt thereof, for use in treatment of a KRAS G12C mutant cancer, wherein the compound of Formula I, or a pharmaceutically acceptable salt thereof, is administered at a dose of about 100 mg.

In another aspect, disclosed herein is the compound of Formula I, or a pharmaceutically acceptable salt thereof, for use in treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with a second therapeutic agent.

In another aspect, disclosed herein is the compound of Formula I, or a pharmaceutically acceptable salt thereof, for use in treatment of a KRAS G12C mutant cancer, administering to a patient in need thereof, a dose of about 150 mg of the compound of Formula I, or a pharmaceutically acceptable salt thereof, in simultaneous, separate or sequential combination with a second therapeutic agent.

In another aspect, disclosed herein is the compound of Formula I, or a pharmaceutically acceptable salt thereof, for use in treatment of a KRAS G12C mutant cancer, administering to a patient in need thereof, a dose of about 100 mg of the compound of Formula I, or a pharmaceutically acceptable salt thereof, in simultaneous, separate or sequential combination with a second therapeutic agent.

In another aspect, disclosed herein is the compound of Formula I, or a pharmaceutically acceptable salt thereof, for use in treatment of a KRAS G12C mutant cancer, administering to a patient in need thereof, a dose of about 50 mg of the compound of Formula I, or a pharmaceutically acceptable salt thereof, in simultaneous, separate or sequential combination with a second therapeutic agent.

In another aspect, disclosed herein is the compound of Formula I, or a pharmaceutically acceptable salt thereof, for use in treatment of a KRAS G12C mutant cancer, administering to a patient in need thereof, a dose between about 50 mg and about 200 mg of, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of about 150 mg of, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of about 100 mg of, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of about 50 mg of, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose between of 150 mg of, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 100 mg of, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 50 mg of, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%.

In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50% In another aspect, disclosed herein is the compound of Formula I, or a pharmaceutically acceptable salt thereof, for use in treatment of a KRAS G12C mutant cancer, administering to a patient in need thereof, a dose of 150 mg of, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 100 mg of, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 50 mg of, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose between of 150 mg of, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 100 mg of, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 50 mg of, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 150 mg of, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 100 mg of, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49% In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is the compound of Formula I, or a pharmaceutically acceptable salt thereof, for use in treatment of a KRAS G12C mutant cancer, administering to a patient in need thereof, a dose of 50 mg of, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50% In another aspect, disclosed herein is a use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose between about 50 mg and about 200 mg.

In another aspect, disclosed herein is a use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose of about 150 mg.

In another aspect, disclosed herein is a use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose of about 100 mg.

In another aspect, disclosed herein is a use of a compound of Formula I, or a pharmaceutically acceptable salt thereof, in the manufacture of a medicament for treatment of a KRAS G12C mutant cancer, wherein
the compound of Formula I, or a pharmaceutically acceptable salt thereof, is administered at a first dose selected from the group consisting of about 50 mg, about 100 mg, about 150 mg, and about 200 mg;
the patient is monitored for DLT; and
if the patient exhibits DLT, a second dose of the compound of Formula I, or a pharmaceutically acceptable salt thereof, is administered, wherein the second dose is reduced as compared to the first dose.

In another aspect, disclosed herein is a use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose of about 150 mg;
the patient is monitored for DLT; and
if the patient exhibits DLT, a second dose of the compound of Formula I, or a pharmaceutically acceptable salt thereof, is administered, wherein the second dose is reduced as compared to the first dose.

In another aspect, disclosed herein is a use of a compound of Formula I, or a pharmaceutically acceptable salt thereof, in the manufacture of a medicament for treatment of a KRAS G12C mutant cancer, wherein
the compound of Formula I, or a pharmaceutically acceptable salt thereof, is administered at a first dose of about 100 mg;
the patient is monitored for DLT; and
if the patient exhibits DLT, a second dose of the compound of Formula I, or a pharmaceutically acceptable salt thereof, is administered, wherein the second dose is reduced as compared to the first dose.

In still another aspect, disclosed herein is a use of the compound of Formula I or a pharmaceutically acceptable salt thereof, in the manufacture of a medicament for treatment of a KRAS G12C mutant cancer, wherein a dose between about 50 mg and about 200 mg of a compound of Formula I or a pharmaceutically acceptable salt thereof is administered to a patient in need thereof, wherein the KRAS G12C mutant cancer is KRAS G12C-mutant advanced NSCLC.

In still another aspect, disclosed herein is a use of the compound of Formula I:

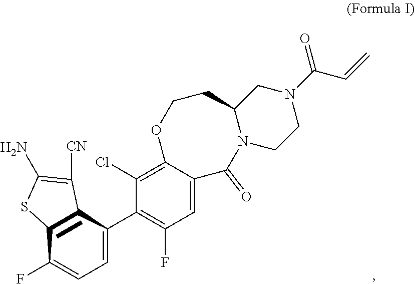

or a pharmaceutically acceptable salt thereof, in the manufacture of a medicament for treatment of a KRAS G12C mutant cancer, wherein a dose of about 150 mg of a compound of Formula I or a pharmaceutically acceptable salt thereof is administered to a patient in need thereof, wherein the KRAS G12C mutant cancer is KRAS G12C-mutant advanced NSCLC.

In another aspect, disclosed herein is use of the compound of Formula I, or a pharmaceutically acceptable salt thereof, in the manufacture of a medicament for treatment of a KRAS G12C mutant cancer, wherein the compound, is administered at a first dose selected from the group consisting of about 50 mg, about 100 mg, about 150 mg, and about 200 mg; the patient is monitored for DLT; and if the patient exhibits DLT, a second dose of the compound of Formula I, or a pharmaceutically acceptable salt thereof, is administered, wherein the second dose is reduced as compared to the first dose.

In another aspect, disclosed herein is the compound of Formula I, or a pharmaceutically acceptable salt thereof, for use in treatment of a KRAS G12C mutant cancer, wherein the compound of Formula I, or a pharmaceutically acceptable salt thereof, is administered at a first dose of about 150 mg; the patient is monitored for DLT; and if the patient exhibits DLT, a second dose of the compound of Formula I, or a pharmaceutically acceptable salt thereof, is administered, wherein the second dose is reduced as compared to the first dose. In one embodiment, the KRAS G12C mutant cancer is selected from the group consisting of KRAS G12C-mutant advanced NSCLC, KRAS G12C-mutant lung cancer, KRAS G12C-mutant colorectal cancer, KRAS G12C-mutant pancreatic cancer, KRAS G12C-mutant bladder cancer, KRAS G12C-mutant cervical cancer, KRAS G12C-mutant endometrial cancer, KRAS G12C-mutant ovarian cancer, KRAS G12C-mutant cholangiocarcinoma, and KRAS G12C-mutant esophageal cancer. In another embodiment, the KRAS G12C mutant cancer is selected from the group consisting of KRAS G12C-mutant non-small cell lung cancer, KRAS G12C-mutant pancreatic cancer, or KRAS G12C-mutant colorectal cancer. In another embodiment, the KRAS G12C mutant cancer is KRAS G12C-mutant non-small cell lung cancer. In another embodiment, the KRAS G12C mutant cancer is KRAS G12C-mutant pancreatic cancer. In another embodiment, the KRAS G12C mutant cancer is KRAS G12C-mutant colorectal cancer.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with a second therapeutic agent.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with a second therapeutic agent.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with a second therapeutic agent.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with a second therapeutic agent.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with abemaciclib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between of 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC.

In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between of 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100% In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49% In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, cisplatin is dosed (or administered) 75 mg/m2 once every three weeks. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In an embodiment, pembrolizumab is administered for up to thirty-five cycles. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In yet another embodiment, pemetrexed is dosed (or administered) 500 mg/m2 once every three weeks. In yet another embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced non-squamous NSCLC. In an embodiment, pembrolizumab is dosed (or administered) 200 mg once every three weeks. In another embodiment, pemetrexed is dosed (or administered) 500 mg/m2 once every three weeks. In yet another embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In yet another embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with erlotinib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 150 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 100 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg of the compound of Formula I, or a pharmaceutically acceptable salt thereof, in simultaneous, separate or sequential combination with Temuterkib in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 150 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 100 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg of the compound of Formula I, or a pharmaceutically acceptable salt thereof, in simultaneous, separate or sequential combination with Temuterkib in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid: 2-methylpropan-2-amine (1:1) salt in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced NSCLC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose between about 50 mg and about 200 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with cetuximab in the treatment of KRAS G12C-mutant advanced CRC.

In another aspect, disclosed herein is a use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with cetuximab in the treatment of KRAS G12C-mutant advanced CRC.

### BRIEF DESCRIPTION OF THE FIGURES

FIG. 1 is KRAS G12C Background.

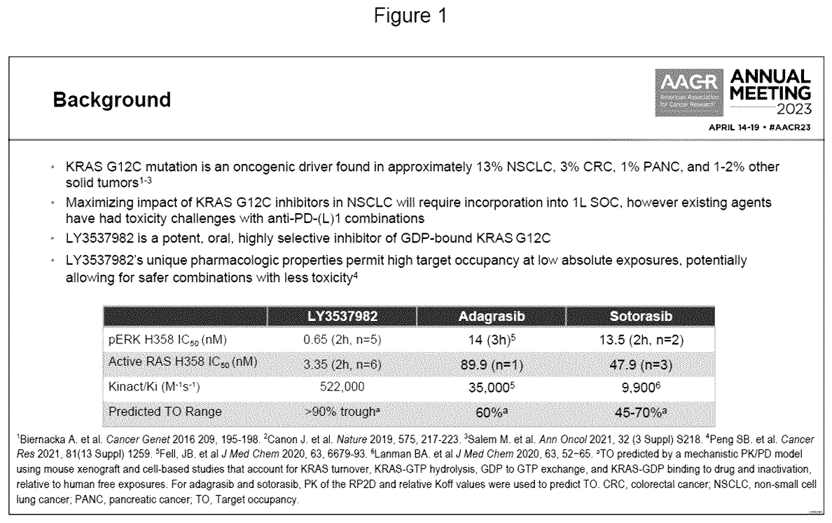

FIG. 2 is LOXO-RAS-20001 Study Eligibility, Design, Objectives.

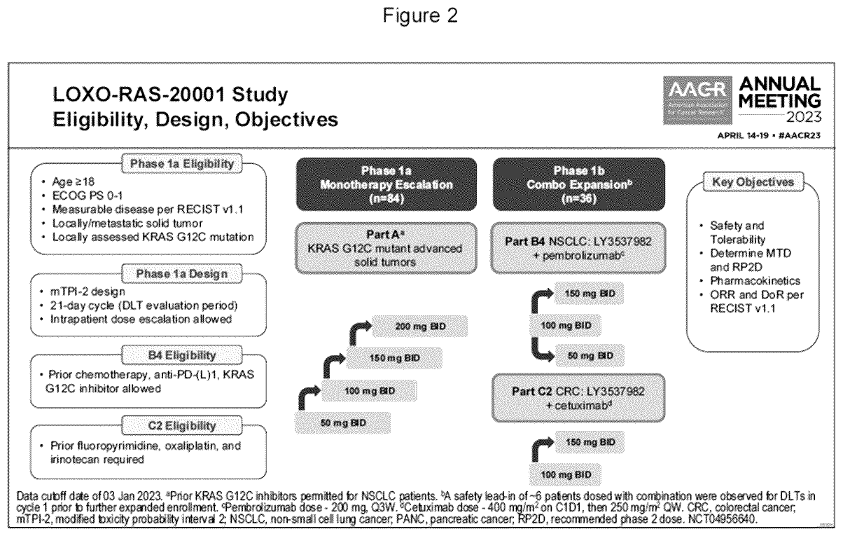

FIG. 3 is Patient and Disease Characteristics—Monotherapy.

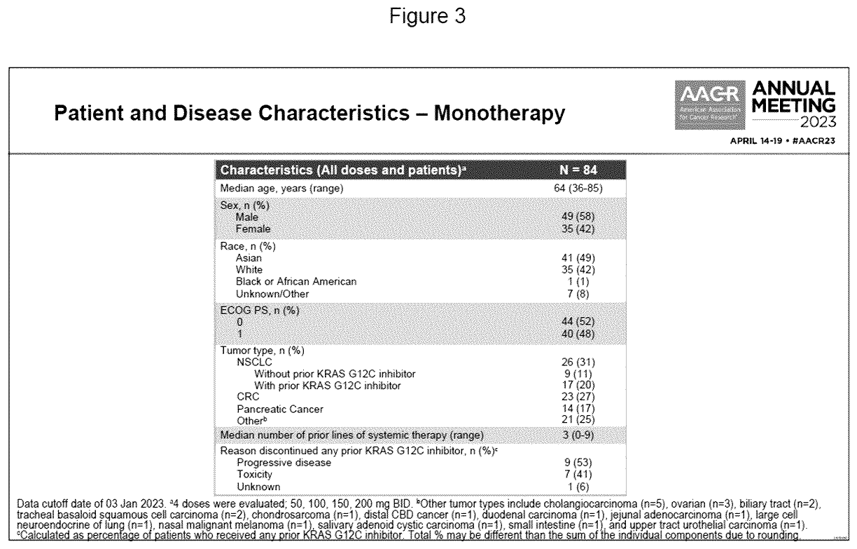

FIG. 4 is LY3537982 Pharmacokinetics.

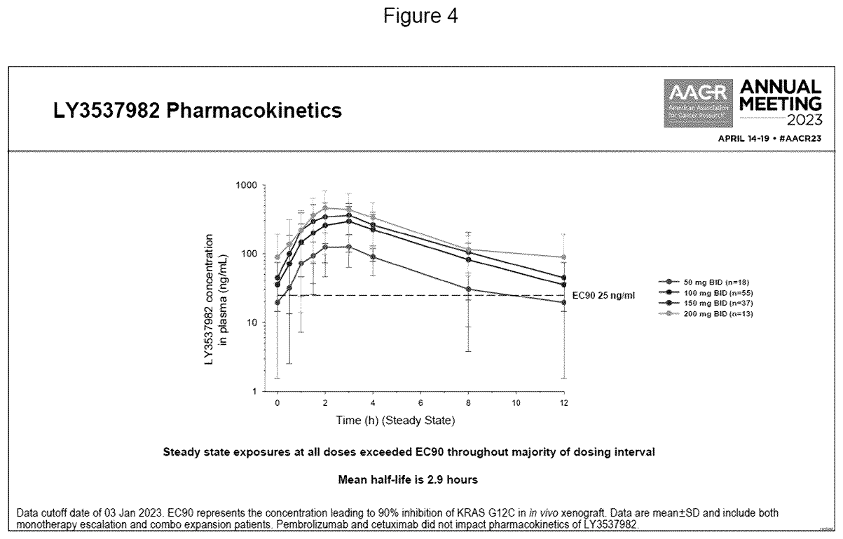

FIG. 5 is Safety Profile—Monotherapy Escalation.

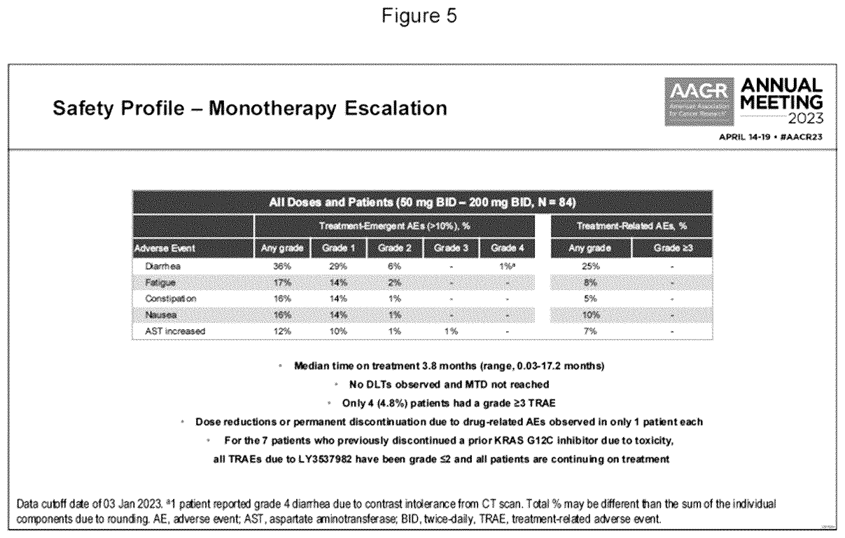

FIG. 5A is Safety Profile—Monotherapy Escalation. 50 mg BID has the lowest LY3537982 concentration in plasma. 100 mg BID is the second lowest. 150 mg BID is the second highest. 200 mg BID has the highest LY3537982 concentration in plasma.

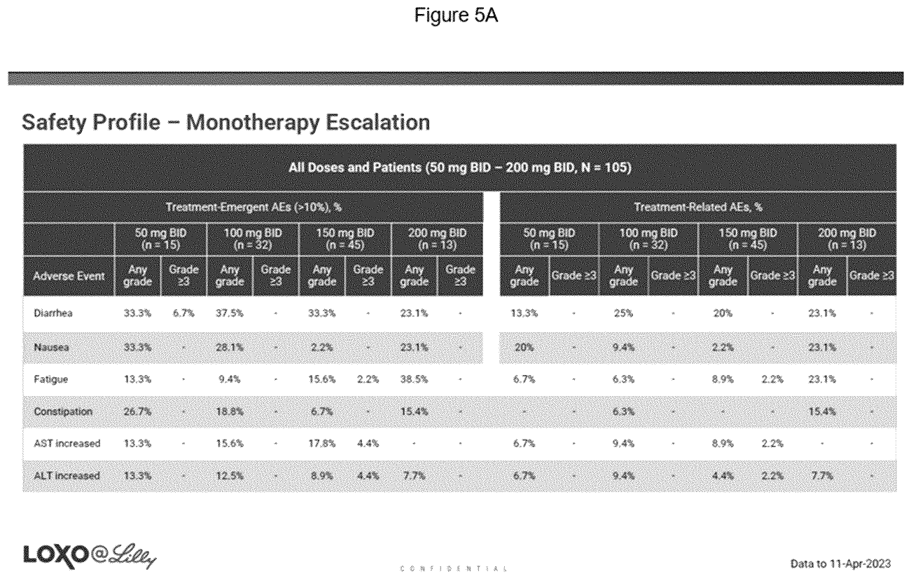

FIG. 6 is Efficacy of LY3537982—Monotherapy Escalation.

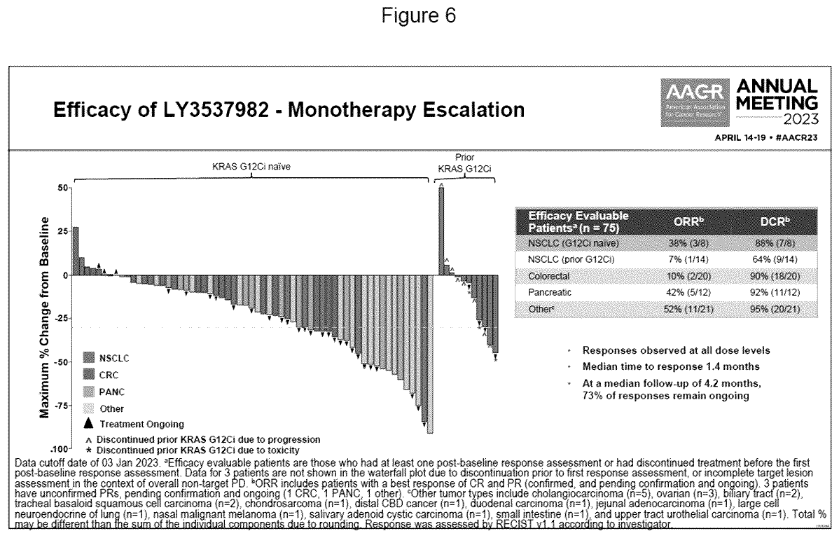

FIG. 7 is NSCLC Patient and Disease Characteristics, LY3537982+Pembrolizumab Combination (Cohort B4).

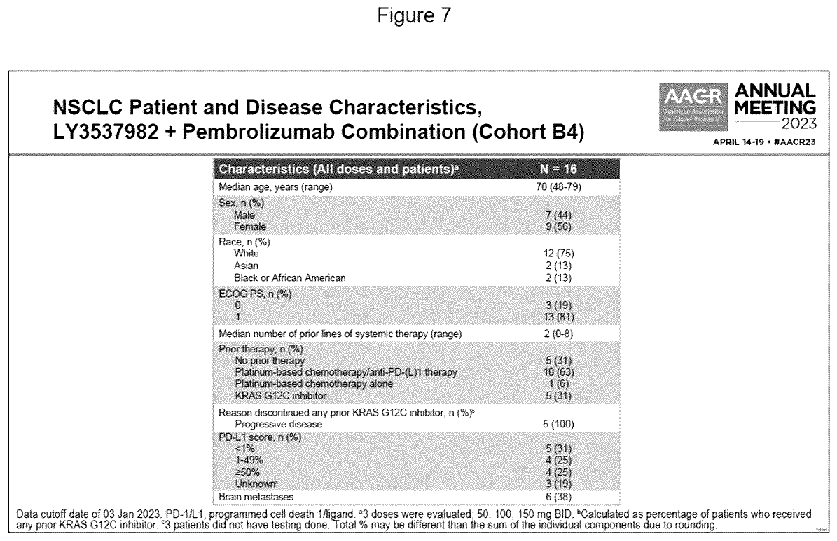

FIG. 8 is Safety Profile in NSCLC LY3537982+Pembrolizumab Combination (Cohort B4).

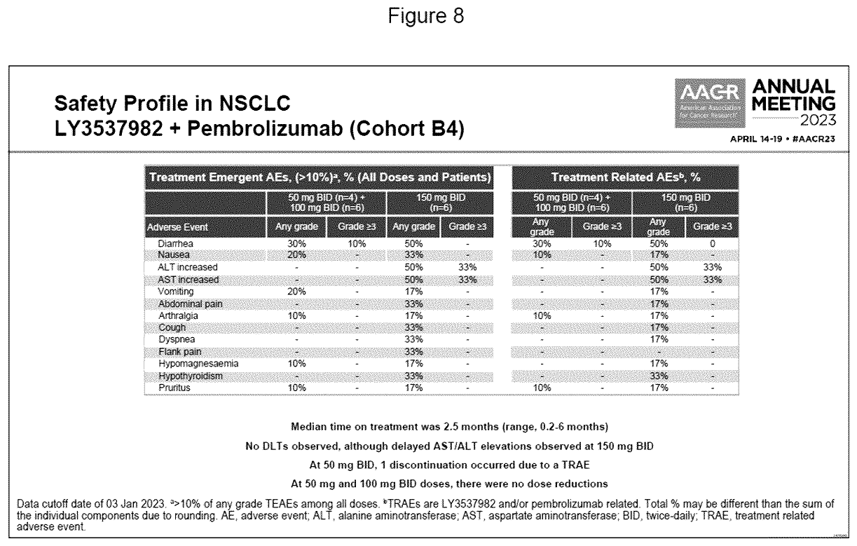

FIG. 8A is Safety Profile in NSCLC LY3537982+Pembrolizumab Combination (Cohort B4).

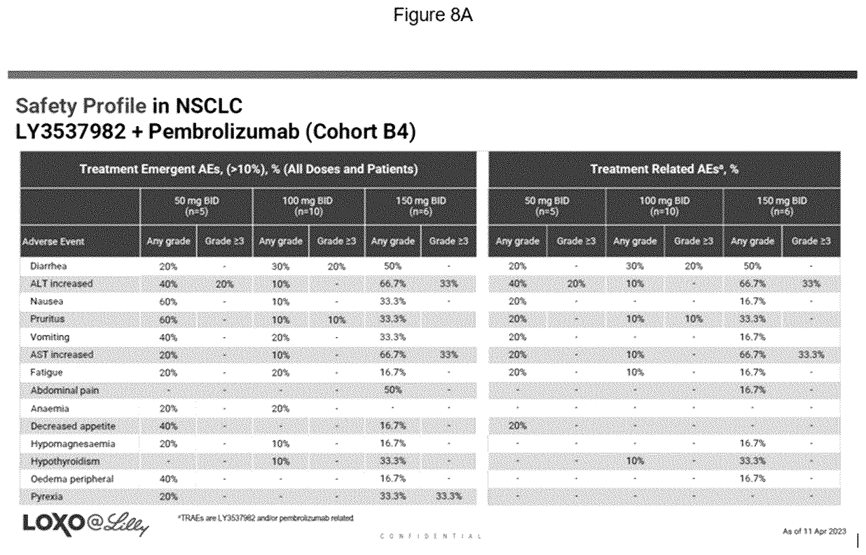

FIG. 9 is Safety Profile in NSCLC LY3537982+Pembrolizumab Combination (Cohort B4).

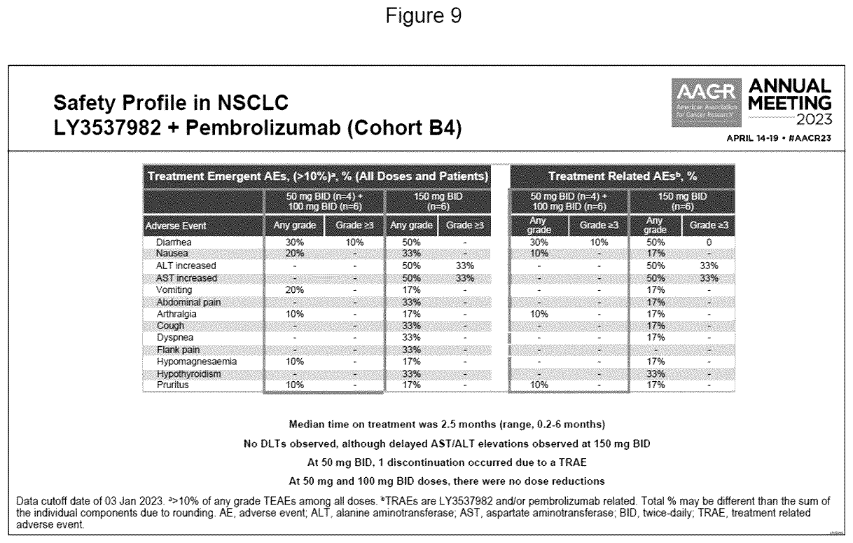

FIG. 10 is Efficacy and Treatment Duration in NSCLC LY3537982+Pembrolizumab Combination (Cohort B4).

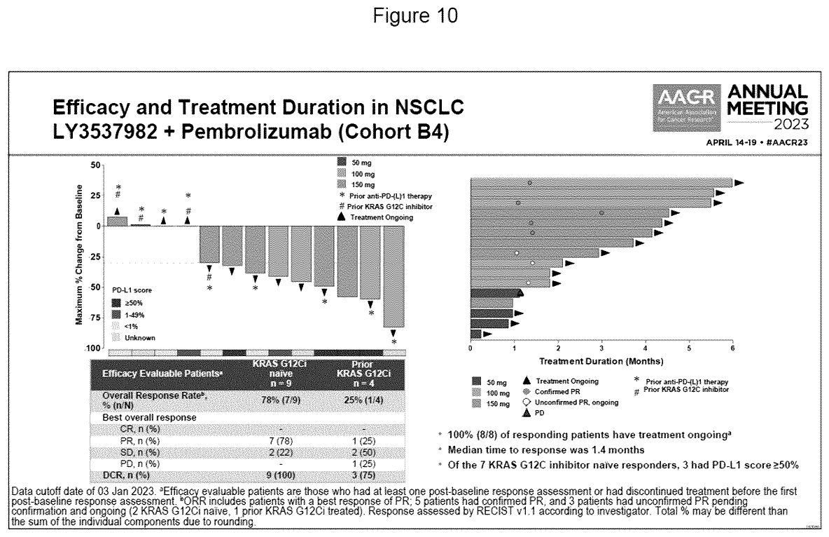

FIG. 11 is CRC Patient and Disease Characteristics, LY3537982+Cetuximab Combination (Cohort C2).

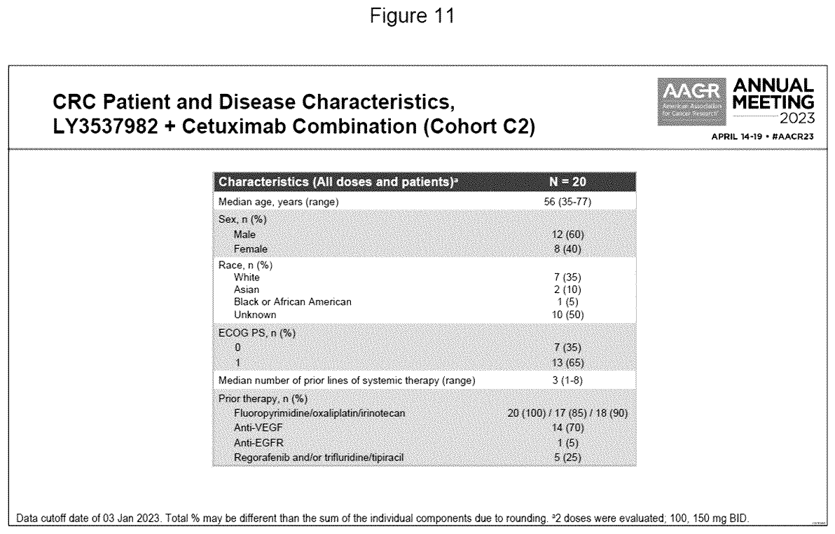

FIG. 12 is Safety Profile in CRC LY3537982+Cetuximab Combination (Cohort C2).

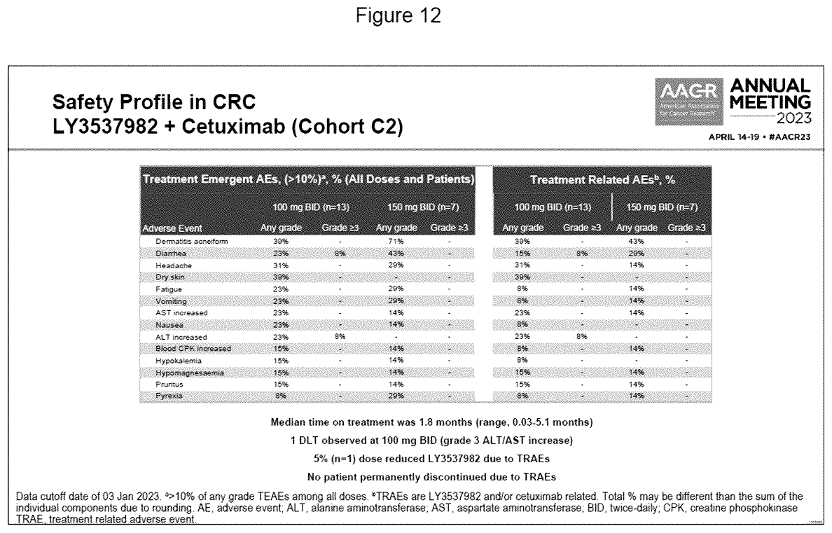

FIG. 13 is Efficacy and Treatment Duration in CRC LY3537982+Cetuximab Combination (Cohort C2).

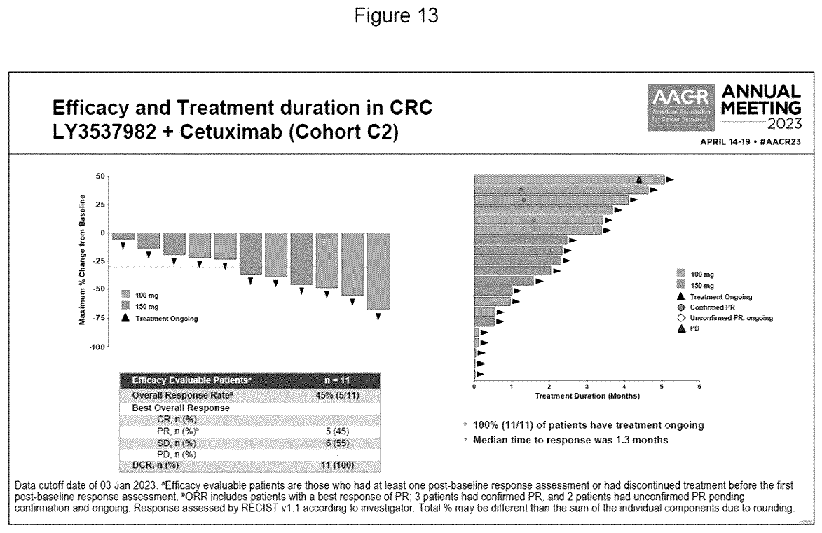

FIG. 14 is LOXO-RAS-20001 Trial in Progress Conclusions.

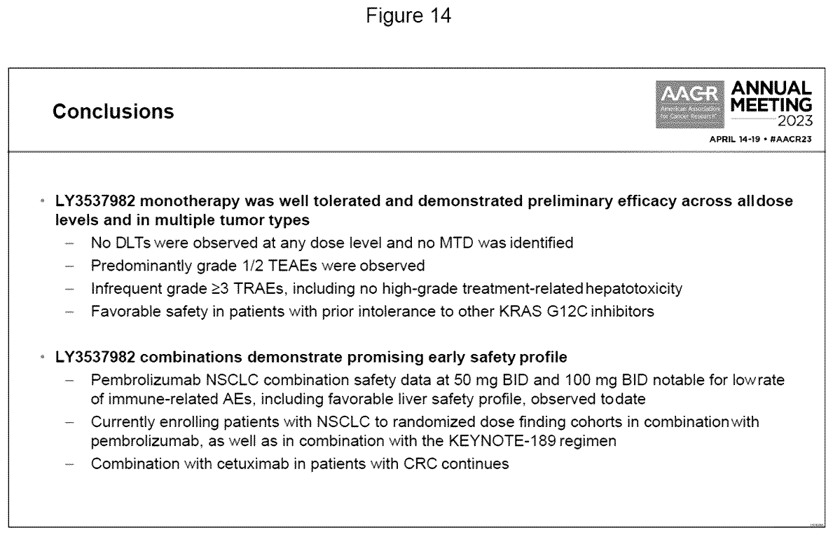

### DETAILED DESCRIPTION

Methods and uses that utilize the compound of Formula I or pharmaceutically acceptable salts thereof are described below.

### Definitions

The terms “+14 days” or “+14 day window” as used herein refers to an additional 14 day period of time to the number of days of treatment cycle. For example, a patient's one 21-day cycle of pembrolizumab must have been initiated within 35 days prior to administering to a patient in need thereof, a dose of a compound of Formula I. To be clear, in this embodiment, the cycle of pembrolizumab must have been initiated within 35 days of administering to a patient in need thereof, a dose of the compound of Formula I, or a pharmaceutically acceptable salt thereof.

The 95% confidence interval was calculated using the Clopper-Pearson method.

The term “BOR” as used herein refers to best overall response based on investigator assessments using RECIST criteria (version 1.1). The minimum duration for BOR of SD is at least 6 weeks after the date of first dose of study drug.

The term “CR” as used herein refers to complete response.

The term “CR Confirmed” as used herein refers to a confirmed complete response, wherein confirmation of CR is required at least 4 weeks after the date of initial documentation of response.

The term “cycle” as used herein refers to treatment cycles. In both Phase 1a and Phase 1b, each treatment cycle is 21 days. To be clear, in an embodiment, pembrolizumab is administered for no more than thirty-five cycles. In this embodiment, pembrolizumab is administered for up to four cycles in simultaneous, separate or sequential combination with the compound of Formula I, or a pharmaceutically acceptable salt thereof, pemetrexed, and cisplatin or carboplatin. To be clear, in this embodiment cisplatin or carboplatin are no longer administered after four cycles. In this embodiment, pembrolizumab is administered for up to an additional thirty-one cycles in simultaneous, separate or sequential combination with pemetrexed. In this embodiment, the additional thirty-one cycles of the compound of Formula I, or a pharmaceutically acceptable salt thereof, pembrolizumab and pemetrexed is in addition to the four cycles of the compound of Formula I, or a pharmaceutically acceptable salt thereof, pembrolizumab pemetrexed, and cisplatin or carboplatin resulting in no more than thirty-five cycles of pembrolizumab.

The disease control rate is the proportion of patients with best overall response of CR, PR, uPR, or SD.

The term “DOR” as used herein refers to duration of response, wherein DOR in months is calculated as the event or censoring date minus the response start date plus 1 day divided by 30.4375.

Efficacy evaluable analysis set is defined as all patients who had a baseline and greater than or equal to one post-baseline disease assessment or discontinued study treatment prior to the first post-baseline assessment.

The term “MTD” as used herein refers to maximum tolerable dose.

The term “NE” as used herein refers to not estimable.

The term “QT prolongation” as used herein refers to a measure of delayed ventricular repolarization (i.e., lengthening of the time between the start of the Q wave and the end of the T wave in an electrocardiogram measurement).

The term “ORR” as used herein refers to objective response rate, wherein objective response rate is the proportion of patients with BOR of CR or PR. Objective response rate, unconfirmed, is the proportion of patients with BOR of CR, PR, or uPR.

Overall response status is the status as of patient's last disease assessment on or before a data cutoff date.

The term “PD” as used herein refers to progressive disease.

The term “Pd” as used herein refers to palladium.

The term “PDX” as used herein refers to patient-derived xenograft.

The term “PD-1” as used herein refers to programmed death receptor 1.

Pembrolizumab, Nivolumab, Cemiplimab, Dostarlimab, and Retifanlimab are each PD-1 inhibitors.

The term “PD-L1” as used herein refers to programmed death ligand 1. Atezolizumab, Avelumab, and Durvalumab are each PD-L1 inhibitors.

The term “PR” as used herein refers to partial response.

The term “uPR” as used herein refers to a partial response pending confirmation, wherein confirmation of PR is required at least 4 weeks after the date of initial documentation of response.

The term “PR Confirmed” as used herein refers to a confirmed partial response, wherein confirmation of PR is required at least 4 weeks after the date of initial documentation of response.

The term “RP2D” as used herein refers to recommended Phase 2 dose. It is understood that RP2D could refer to the recommended Phase 2 dose for each of monotherapy or combination therapy.

The term “RP2DM” as used herein refers to recommended Phase 2 dose monotherapy.

The term “SD” as used herein refers to stable disease.

The term “TPS” as used herein refers to tumor proportion score. For example, a patient may have a PD-L1-positive (TPS ≥1%) tumor as determined by IHC using anti-PD-L1 antibody clone 22C3 at a local or sponsor-designated laboratory with CLIA, ISO/IEC, CAP, or other similar certification as per local guidelines including, but not limited to, IVDR compliance as applicable.

The term “TRAE” as used herein refers to treatment-related adverse event.

The term “TTR” as used herein refers to time to response, wherein TTR in months is calculated as response start date minus first dose date plus 1 day divided by 30.4375.

The term “ILD” as used herein refers to interstitial lung disease.

The term “AUC” as used herein refers to the area under the concentration versus time curve.

The term “AST/ALT ratio” as used herein refers to the ratio between the concentrations of the enzymes aspartate transaminase (AST) and alanine transaminase, aka alanine aminotransferase (ALT) in the blood of a human or animal.

The term “HBsAg” as used herein refers to Hepatitis B surface antigen.

The term “HBV” as used herein refers to Hepatitis B virus.

The term “AE” as used herein refers to adverse event.

The term “irAE” as used herein refers to immune-related AE.

The term “Gy” as used herein refers to the total amount of radiation exposure for a patient.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19, Gould, P. L., “Salt selection for basic drugs,” International Journal of Pharmaceutics, 3: 201-217 (1986); Bastin, R. J., et al. “Salt Selection and Optimization Procedures for Pharmaceutical New Chemical Entities,” Organic Process Research and Development, 4: 427-435 (2000).

As used herein, the term “effective amount” refers to an amount that is a dose, which is effective in treating a disorder or disease, such as a cancerous lesion or progression of abnormal cell growth and/or cell division. Factors considered in the determination of an effective amount or dose of a compound include: whether the compound or its salt will be administered; the co-administration of other agents, if used; the species of patient to be treated; the patient's size, age, and general health; the degree of involvement or stage and/or the severity of the disorder; the response of the individual patient; the mode of administration; the bioavailability characteristics of the preparation administered; the dose regimen selected; and the use of other concomitant medication.

Preferred pharmaceutical compositions can be formulated as a capsule for oral administration. A capsule can include the compound of Formula I, or a pharmaceutically acceptable salt thereof, in an amount effective for treating a patient in need of treatment for cancer.

As used herein, the terms “treating”, “to treat”, or “treatment”, includes slowing, reducing, or reversing the progression or severity of an existing symptom, disorder, condition, which can include specifically slowing the growth of a cancerous lesion or progression of abnormal cell growth and/or cell division.

As used herein, the term “patient” refers to a mammal in need of treatment. Preferably, the patient is a human that is in need of treatment for cancer, for example, KRas G12C mutant bearing cancers.

The term “pediatric patient” as used herein refers to a patient under the age of 21 years at the time of diagnosis or treatment.

The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In one embodiment, the patient is a human that has not received prior KRAS G12C inhibitor (KRAS G12Ci) therapy and is described herein as naïve to KRAS G12Ci. Patients may have received other therapies, including surgery or other pharmaceuticals, but not prior KRAS G12Ci therapy.

In one preferred embodiment, the patient is a human that has received at least one treatment, including prior KRAS G12C inhibitor. The patients may have received an unlimited number of other therapies, including surgery or one other prior therapy, or two other prior therapies, three other prior therapies or four or more other prior therapies.

Prior KRAS G12Ci therapy requires the patient to have previously had prior treatment with a KRAS G12Ci, such as sotorasib or adagrasib.

Certain abbreviations are defined as follows: “DMEM” refers to Dulbecco's modified Eagle's medium; “DPEPhosPdCl₂” refers to dichlorobis(diphenylphophinophenyl)ether palladium (II); “ERK” refers to extracellular signal-regulated kinases; “HPLC” refers to high-performance liquid chromatography; and “PCR” refers to polymerase chain reaction.

Salts

A compound of Formula I is readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group.

Form

A compound of Formula I may be in the form of, for example, a free base, or a pharmaceutically acceptable salt thereof. In an embodiment, a compound of Formula I is not a pharmaceutically acceptable salt, for example, it is a free base. In an alternate embodiment, a compound of Formula I is a pharmaceutically acceptable salt. In a preferred embodiment, the compound of Formula I is a free base.

For the avoidance of doubt, when a dose is described herein, the dose refers to the amount of the free base, for example, the non-salt version of the compound of Formula I that is administered. For example, a 200 mg dose would require more than 200 mg of a pharmaceutically acceptable salt of the compound of Formula I, because the weight of the salt counterion must be included.

Cancer

Cancers that may be treated using the methods and protocols described herein include those that are treatable by inhibiting KRAS G12C. In other embodiments, the cancer has one or more cancer cells that express the mutant KRas G12C protein.

Examples of these cancers include but are not limited to lung cancer, colorectal cancer, pancreatic cancer, bladder cancer, cervical cancer, endometrial cancer, ovarian cancer, cholangiocarcinoma, and esophageal cancer.

In preferred embodiments, the cancer is non-small cell lung cancer, pancreatic cancer, or colorectal cancer. In still more preferred embodiments, the cancer is non-small cell lung cancer or colorectal cancer. In still more preferred embodiments, the cancer is non-small cell lung cancer. In still more preferred embodiments, the cancer is pancreatic cancer. In still more preferred embodiments, the cancer is colorectal cancer.

Dose

In some embodiments, the dose is between about 5 mg to about 250 mg or about 25 mg to about 250 mg, or about 50 mg to about 200 mg. In some embodiments, the dose is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg of a compound of Formula I or a pharmaceutically acceptable salt thereof. In one preferred embodiment, the dose is about 50 mg, about 100 mg, about 150 mg, or about 200 mg. In an embodiment, the dose is about 50 mg. In an embodiment, the dose is about 100 mg. In an embodiment, the dose is about 150 mg. In an embodiment, the dose is about 200 mg. In another embodiment, the dose is about 5 mg of the compound of Formula I, or a pharmaceutically acceptable salt thereof. In an embodiment, the dose is about 50 mg of the compound of Formula I, or a pharmaceutically acceptable salt thereof.

The term “dose” as used herein refers to the total amount of the compound of Formula I, or a pharmaceutically acceptable salt thereof, that is administered at one time.

Dosing Frequency (or Dosage)

The term “dosage” refers to a dose administered at a specific frequency.

The compound may be administered as needed. Typically, the administration is once a day, twice a day, three times a day, or four times a day. In a preferred embodiment, the administration is administered BID, which is twice a day. Commonly, when administered twice a day, both doses are the same strength, e.g., both doses are 50 mg, both doses are 100 mg, both doses are 150 mg, or both doses are 200 mg. But as described elsewhere herein, the doses may be different, for example if the patient exhibits a DLT. In such as case, the second dose would be reduced, relative to the first dose. Alternatively, the first and second doses may be lower than the first and second doses that were administered on a previous day. For example, if about 150 mg was administered as the first and second doses on a day 15 of treatment, but the first and second doses on day 16 of treatment may be reduced relative to the doses on day 15; e.g., the first and second doses on day 16 could both be about 100 mg.

In an embodiment, the step of administering the dose occurs up to the end of the patient's life.

Maximum Daily Dose

The term “maximum daily dose” as used herein refers to the total amount of the compound of Formula I, or a pharmaceutically acceptable salt thereof, that is taken within a 24 hour period. The maximum daily dose depends on the dose administered and the dosing frequency. For example, the maximum daily dose can be 50 mg, 100 mg, 150 mg, 200 mg, 300 mg, 400 mg, 500 mg, 600 mg, 700 mg, or 800 mg. For example, if 50 mg was administered once a day, then the maximum daily dose for that day is 50 mg. If 100 mg was administered twice a day, then the maximum daily dose for that day is 200 mg. If 150 mg was administered three times a day, then the maximum daily dose for that day is 450 mg. If 200 mg was administered four times a day, then the maximum daily dose for that day is 800 mg.

Dose Limiting Toxicity (DLT)

A “DLT” is defined as any of the treatment-emergent adverse event (TEAEs), as defined by the National Cancer Institute's Common Terminology Criteria for Adverse Events, version 5.0 (NCI CTCAE v5.0), deemed clinically significant and occurring during the first 21 days of study drug per dose level per patient, unless the adverse event (AE) can be clearly related to the patient's underlying disease, other medical condition, or concomitant medications including any AEs attributed by the Investigator to the combination agent alone. A TEAE is defined as an AE that starts or worsens on or after the date of the first dose of study drug (for example on Study Day 1) through 28 days (+14 days window) after the date of the last dose of study drug or the first date starting new anticancer therapy, whichever is earlier. A Serious TEAE is defined as an TEAE that is grade 3 or higher based on severity grade assignment as defined by the National Cancer Institute's Common Terminology Criteria for Adverse Events, version 5.0 (NCI CTCAE v5.0). A Fatal TEAE is defined as an TEAE that is grade 5 based on severity grade assignment as defined by the National Cancer Institute's Common Terminology Criteria for Adverse Events, version 5.0 (NCI CTCAE v5.0).

In an embodiment, the treatment-emergent adverse event is hematologic toxicity, febrile neutropenia, hepatotoxicity, mucositis, diarrhea, ocular toxicity, QT prolongation, confirmed ILD, pneumonitis, constipation, nausea or vomiting, fatigue, AST/ALT ratio, creatinine elevations, elevated or increased bilirubin, neutropenia, anemia, or thrombocytopenia.

If the patient exhibits one or more symptoms, such as toxicity, a clinically significant adverse event, intolerability, and a drug-drug interaction, the dose of the drug may be reduced. The term “first dose” as used herein regarding DLT refers to a dose of the compound of Formula I, or a pharmaceutically acceptable salt thereof, that is administered prior to a second or subsequent dose. In an embodiment, it is not required that the first dose refer to only the first time a patient is administered a dose of the compound of Formula I, or a pharmaceutically acceptable salt thereof. For example, the first dose could refer to the third or fifth dose administered to a patient so long as the first dose precedes the patient experiencing a DLT. In an embodiment, the patient exhibits a clinically significant adverse event and the second and/or subsequent doses, are reduced, relative to the dose that was administered before the TEAE was identified.

Reducing the dose should help to alleviate one or more symptoms, and preferably, help the patient stay in treatment. When one or more symptoms is exhibited, each dose is typically reduced by about 200 mg or about 150 mg or about 100 mg or about 50 mg. Of course, the dose cannot be reduced by an amount that is greater than the original dose. For example, if a patient is receiving a 150 mg dose BID, it is impossible to reduce these doses by 200 mg. In such a case, the most the dose could be reduced would be about 100 mg or about 50 mg. In some embodiments, the second daily dose is reduced, as compared to the first daily dose. In one embodiment, the second daily dose is reduced by 50 mg as compared to the first daily dose. In another embodiment, the first and second daily doses are reduced as compared to the first and second daily doses administered on a prior day.

In an embodiment, the second dose is reduced by 50 mg as compared to the first dose except for when the first dose is 50 mg, when the first dose is 50 mg, the second dose is reduced by 5 mg, 10 mg, 15 mg, 20 mg, 25 mg, 30 mg, 35 mg, 40 mg, or 45 mg. In another embodiment, the second dose is reduced by 100 mg as compared to the first dose except for when the first dose is 50 mg or 100 mg, when the first dose is 50 mg, the second dose is reduced by 5 mg, 10 mg, 15 mg, 20 mg, 25 mg, 30 mg, 35 mg, 40 mg, or 45 mg, when the first dose is 100 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg. In an embodiment, the second dose is reduced by 150 mg as compared to the first dose except for when the first dose is 50 mg, 100 mg or 150 mg, when the first dose is 50 mg, the second dose is reduced by 5 mg, 10 mg, 15 mg, 20 mg, 25 mg, 30 mg, 35 mg, 40 mg, or 45 mg, when the first dose is 100 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg, when the first dose is 150 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, or 145 mg.

If needed, the second daily dose can be reduced to a third daily dose. When one or more symptoms are exhibited, the second daily dose is typically reduced to a third daily dose. The second daily dose may be reduced by about 200 mg or about 150 mg or about 100 mg or about 50 mg. As before, the dose cannot be reduced by an amount that is greater than the original dose. In a preferred embodiment, the third daily dose is about 50 mg less than the second daily dose. To be clear, the third daily dose is not necessarily the third dose in a single day. Rather, it is the dose that is administered if the second daily dose causes a symptom or otherwise needs to be decreased whether on the same day or on a subsequent day. For example, if a patient receives a 150 mg dose BID that results in DLT, a second daily dose of 100 mg BID may be administered. If a patient is receiving a 100 mg dose BID that results in DLT, a third daily dose of 50 mg BID may be administered.

Dose Reduction

Also disclosed herein is a method of treating a KRAS G12C mutant cancer, comprising administering to a patient in need of such treatment, a first dose between about 50 mg and about 200 mg of the compound of Formula I, or a pharmaceutically acceptable salt thereof; monitoring the patient for a DLT; and administering a second dose of the compound of Formula I, or a pharmaceutically acceptable salt thereof, if the patient exhibits the DLT, wherein the second dose is reduced as compared to the first dose. In an embodiment, the first dose is selected from the group consisting of about 50 mg, about 100 mg, about 150 mg, and about 200 mg. In an embodiment, the second dose is selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, and about 150 mg.

In another aspect, disclosed herein is a method of treating a KRAS G12C mutant cancer comprising administering to a patient in need of such treatment a second dose selected from the group consisting of about 50 mg, about 100 mg, about 150 mg, and about 200 mg of the compound of Formula I, or a pharmaceutically acceptable salt thereof, monitoring the patient for a DLT; and administering a third dose of the compound of Formula I, or a pharmaceutically acceptable salt thereof, if the patient exhibits a DLT, wherein the third dose is reduced as compared to the second dose. In an embodiment, the third dose is reduced by 50 mg as compared to the second dose.

In another aspect, disclosed herein is a use of the compound of Formula I, or a pharmaceutically acceptable salt thereof, administering to a patient in need thereof, a dose between about 50 mg and about 200 mg of the compound of Formula I, or a pharmaceutically acceptable salt thereof, in simultaneous, separate or sequential combination with a second therapeutic agent, wherein the dose of the compound of Formula I, or a pharmaceutically acceptable salt thereof, is a reduced dose selected from the group consisting of about 50 mg, about 100 mg, and about 150 mg.

Methods

The methods of treating the cancers described herein comprise administering the doses described herein with the dosing frequencies described herein. Specific examples of the methods are described below.

In one aspect, disclosed herein is a method of treating a KRAS G12C mutant cancer, comprising administering to a patient in need thereof, a dose of about 50 mg of the compound of Formula I, or a pharmaceutically acceptable salt thereof, in which the cancer has one or more cells that express a mutant KRas G12C protein. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in which the cancer has one or more cells that express a mutant KRas G12C protein. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day.

In another aspect, disclosed herein is a method of treating cancer, comprising administering to a patient in need thereof, a dose of about 150 mg of the compound of Formula I, or a pharmaceutically acceptable salt thereof, in which the cancer has one or more cells that express a mutant KRas G12C protein. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day.

In another aspect, disclosed herein is a method of treating a KRAS G12C mutant cancer comprising administering to a patient in need of such treatment, a dose of about 150 mg of compound of Formula I, or a pharmaceutically acceptable salt thereof. In one embodiment, the KRAS G12C mutant cancer is selected from the group consisting of KRAS G12C-mutant advanced NSCLC, KRAS G12C-mutant lung cancer, KRAS G12C-mutant colorectal cancer, KRAS G12C-mutant pancreatic cancer, KRAS G12C-mutant bladder cancer, KRAS G12C-mutant cervical cancer, KRAS G12C-mutant endometrial cancer, KRAS G12C-mutant ovarian cancer, KRAS G12C-mutant cholangiocarcinoma, and KRAS G12C-mutant esophageal cancer. In another embodiment, the KRAS G12C mutant cancer is selected from the group consisting of KRAS G12C-mutant non-small cell lung cancer, KRAS G12C-mutant pancreatic cancer, or KRAS G12C-mutant colorectal cancer. In another embodiment, the KRAS G12C mutant cancer is KRAS G12C-mutant non-small cell lung cancer. In another embodiment, the KRAS G12C mutant cancer is KRAS G12C-mutant pancreatic cancer. In another embodiment, the KRAS G12C mutant cancer is KRAS G12C-mutant colorectal cancer.

In another aspect, disclosed herein is a method of treating a KRAS G12C mutant cancer, comprising administering to a patient in need thereof, a dose of about 200 mg of the compound of Formula I, or a pharmaceutically acceptable salt thereof, in which the cancer has one or more cells that express a mutant KRas G12C protein. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, wherein after the administration of the compound of Formula I, or a pharmaceutically acceptable salt thereof, the patient achieves a partial response (PR).

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, wherein after the administration of the compound of Formula I, or a pharmaceutically acceptable salt thereof, the patient achieves a complete response.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, wherein after the administration of the compound of Formula I, or a pharmaceutically acceptable salt thereof, the patient achieves a stable disease (“SD”).

In yet another aspect, disclosed herein is a method of treating a KRAS G12C mutant cancer, comprising administering to a patient in need of such treatment, a first dose of about 150 mg of the compound of Formula I, or a pharmaceutically acceptable salt thereof, monitoring the patient for a DLT; and administering a second dose of the compound, or a pharmaceutically acceptable salt thereof, if the patient exhibits the DLT, wherein the second dose is reduced as compared to the first dose. In one embodiment, the KRAS G12C mutant cancer is selected from the group consisting of KRAS G12C-mutant advanced NSCLC, KRAS G12C-mutant lung cancer, KRAS G12C-mutant colorectal cancer, KRAS G12C-mutant pancreatic cancer, KRAS G12C-mutant bladder cancer, KRAS G12C-mutant cervical cancer, KRAS G12C-mutant endometrial cancer, KRAS G12C-mutant ovarian cancer, KRAS G12C-mutant cholangiocarcinoma, and KRAS G12C-mutant esophageal cancer. In another embodiment, the KRAS G12C mutant cancer is selected from the group consisting of KRAS G12C-mutant non-small cell lung cancer, KRAS G12C-mutant pancreatic cancer, or KRAS G12C-mutant colorectal cancer. In another embodiment, the KRAS G12C mutant cancer is KRAS G12C-mutant non-small cell lung cancer. In another embodiment, the KRAS G12C mutant cancer is KRAS G12C-mutant pancreatic cancer. In another embodiment, the KRAS G12C mutant cancer is KRAS G12C-mutant colorectal cancer.

In another aspect, disclosed herein is a method of treating a KRAS G12C mutant cancer comprising administering to a patient in need thereof, a dose between about 50 mg and about 200 mg of the compound of Formula I, or a pharmaceutically acceptable salt thereof, in simultaneous, separate or sequential combination with one or more of a second therapeutic agent. In an embodiment the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

In another aspect, disclosed herein is a method of treating a KRAS G12C mutant cancer comprising administering to a patient in need thereof, a dose of about 150 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer comprising administering to a patient in need thereof, a dose of about 100 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer comprising administering to a patient in need thereof, a dose of about 50 mg of the compound of Formula I, or a pharmaceutically acceptable salt thereof, in simultaneous, separate or sequential combination with a second therapeutic agent.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC. In an embodiment, simultaneous, separate or sequential combination includes where the patient has received no more than one previous cycle of pembrolizumab. In an embodiment, pembrolizumab is dosed (or administered) 200 mg once every three weeks. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49% In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50%. In an embodiment, the KRAS G12C-mutant advanced NSCLC has a tumor proportion score (TPS) status of greater than or equal to 50%. In an embodiment, simultaneous, separate or sequential combination includes where the patient has received no more than one previous cycle of pembrolizumab. In an embodiment, pembrolizumab is dosed (or administered) 200 mg once every three weeks. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50%. In an embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is dosed (or administered) 200 mg once every three weeks. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100% In yet another embodiment, the patient in need thereof has a tumor proportion score (TPS) status of 0% to 49% In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50%. In an embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49% In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49% In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49%. In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy. In another embodiment, the patient in need thereof has received at least one treatment, including prior KRAS G12C inhibitor. In yet another embodiment, the patient in need thereof has a tumor proportion score (TPS) status of 0% to 100%. In yet another embodiment, the KRAS G12C-mutant advanced NSCLC has a tumor proportion score (TPS) status of 0% to 100%. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In yet another embodiment, the patient in need thereof has a tumor proportion score (TPS) status of 1% to 49%. In yet another embodiment, the KRAS G12C-mutant advanced NSCLC has a tumor proportion score (TPS) status of 1% to 49% In yet another embodiment, the patient in need thereof has a tumor proportion score (TPS) status of greater than or equal to 50%. In yet another embodiment, the KRAS G12C-mutant advanced NSCLC has a tumor proportion score (TPS) status of greater than or equal to 50%.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is a method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is a method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose selected from the group consisting of about 50 mg, about 100 mg, about 150 mg, and about 200 mg; the patient is monitored for DLT; and if the patient exhibits DLT, a second dose of the compound of Formula I, or a pharmaceutically acceptable salt thereof, is administered, wherein the second dose is reduced as compared to the first dose.

In another aspect, disclosed herein is use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose of about 150 mg; the patient is monitored for DLT; and if the patient exhibits DLT, a second dose of the compound of Formula I, or a pharmaceutically acceptable salt thereof, is administered, wherein the second dose is reduced as compared to the first dose. In one embodiment, the KRAS G12C mutant cancer is selected from the group consisting of KRAS G12C-mutant advanced NSCLC, KRAS G12C-mutant lung cancer, KRAS G12C-mutant colorectal cancer, KRAS G12C-mutant pancreatic cancer, KRAS G12C-mutant bladder cancer, KRAS G12C-mutant cervical cancer, KRAS G12C-mutant endometrial cancer, KRAS G12C-mutant ovarian cancer, KRAS G12C-mutant cholangiocarcinoma, and KRAS G12C-mutant esophageal cancer. In another embodiment, the KRAS G12C mutant cancer is selected from the group consisting of KRAS G12C-mutant non-small cell lung cancer, KRAS G12C-mutant pancreatic cancer, or KRAS G12C-mutant colorectal cancer. In another embodiment, the KRAS G12C mutant cancer is KRAS G12C-mutant non-small cell lung cancer. In another embodiment, the KRAS G12C mutant cancer is KRAS G12C-mutant pancreatic cancer. In another embodiment, the KRAS G12C mutant cancer is KRAS G12C-mutant colorectal cancer.

KRAS G12C Mutation Status

The KRAS G12C mutational status of one or more cancer cells can be determined by a number of assays known in the art. Typically, one or more biopsies containing one or more cancer cells are obtained, and subjected to sequencing and/or polymerase chain reaction (PCR). Circulating cell-free DNA can also be used, e.g. in advanced cancers. Non-limiting examples of sequencing and PCR techniques used to determine the mutational status (e.g. G12C mutational status, in one or more cancer cells or in circulating cell-free DNA) include direct sequencing, next-generation sequencing, reverse transcription polymerase chain reaction (RT-PCR), multiplex PCR, and pyrosequencing and multi-analyte profiling.

Combinations—Methods and Uses

In another aspect, disclosed herein is a method of treating a KRAS G12C mutant cancer comprising administering to a patient in need thereof, a dose between about 50 mg and about 200 mg of the compound of Formula I, or a pharmaceutically acceptable salt thereof, in simultaneous, separate or sequential combination with a second therapeutic agent. In an embodiment, the dose of the compound of Formula I, or a pharmaceutically acceptable salt thereof, is selected from the group consisting of about 50 mg, about 100 mg, about 150 mg, and about 200 mg.

In an embodiment the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

In another aspect, disclosed herein is a method of treating a KRAS G12C mutant cancer comprising: administering to a patient in need thereof, a dose between about 50 mg and about 200 mg of the compound of Formula I, or a pharmaceutically acceptable salt thereof, in simultaneous, separate or sequential combination with a PD-1 or PD-L1 inhibitor, for use in the treatment of a KRAS G12C mutant cancer. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC. In another embodiment, the PD-1 or PD-L1 inhibitor is pembrolizumab, wherein pembrolizumab is dosed (or administered) 200 mg once every three weeks. In another embodiment, the PD-1 or PD-L1 inhibitor is nivolumab. In another embodiment, the PD-1 or PD-L1 inhibitor is cimiplimab. In another embodiment, the PD-1 or PD-L1 inhibitor is sintilimab. In another embodiment, the PD-1 or PD-L1 inhibitor is atezolizumab. In another embodiment, the PD-1 or PD-L1 inhibitor is avelumab. In another embodiment, the PD-1 or PD-L1 inhibitor is durvalumab. In another embodiment, the PD-1 or PD-L1 inhibitor is lodapilimab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC, wherein pembrolizumab is dosed intravenously at 200 mg once every three weeks. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with a CDK4/CDK6 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC. In another embodiment, the CDK4/CDK6 inhibitor is abemaciclib, wherein abemaciclib is administered 150 mg BID. In another embodiment, the CDK4/CDK6 inhibitor is palbociclib. In another embodiment, the CDK4/CDK6 inhibitor is ribociclib. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with an EGFR inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC. In another embodiment, the EGFR inhibitor is erlotinib, wherein erlotinib is administered 150 mg once a day. In another embodiment, the EGFR inhibitor is afatinib. In another embodiment, the EGFR inhibitor is gefitinib. In another embodiment, the EGFR inhibitor is cetuximab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with cetuximab in the treatment of KRAS G12C-mutant advanced CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with an ERK inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC. In another embodiment, the ERK inhibitor is Temuterkib, wherein Temuterkib is administered 400 mg twice a day. In another embodiment, the ERK inhibitor is LTT462. In another embodiment, the ERK inhibitor is KO-947. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with a platinum agent. In another embodiment, the platinum agent is cisplatin. In another embodiment, the platinum agent is carboplatin. In another embodiment, the platinum agent is oxaliplatin. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg about 100 mg about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, for use in simultaneous, separate or sequential combination with an antifolate, for the treatment of a KRAS G12C mutant cancer. In another embodiment, the antifolate is pemetrexed. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and an Aurora A inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC. In another embodiment, the Aurora A inhibitor is an aminopyridine compound, or a pharmaceutically acceptable salt thereof. In another embodiment, the Aurora A inhibitor is an Aurora A selective inhibitor, or a pharmaceutically acceptable salt thereof. In another embodiment, the Aurora A inhibitor is alisertib as described in WO 2008/063525. In another embodiment, the Aurora A inhibitor is a pan Aurora inhibitor, or a pharmaceutically acceptable salt thereof. In another embodiment, the Aurora A inhibitor is tozasertib as described in WO 2004/000833. In another embodiment, the Aurora A inhibitor is danusertib as described in WO 2005/005427. In another embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:

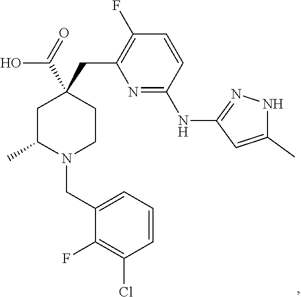

or a pharmaceutically acceptable salt thereof. In an embodiment, the pharmaceutically acceptable salt is an amine salt. One example of the amine salt is NH₃ amine salt. Another example of the amine salt is 2-methylpropan-2-amine salt.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, which has the following structure:

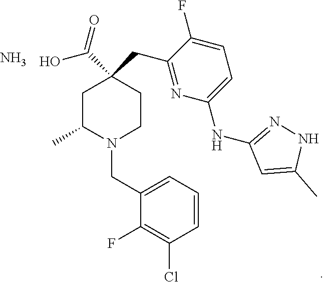

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt, which has the following structure:

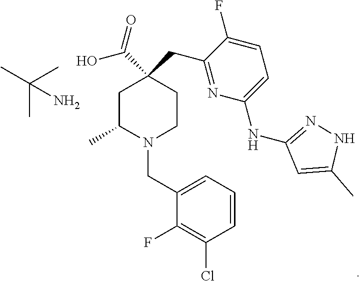

In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and the SHP2 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC. In an embodiment, the SHP2 inhibitor is a Type I SHP2 Inhibitor or a Type II SHP2 Inhibitor. In another embodiment, the Type I SHP2 inhibitor is PHPS1 or GS-493, or a pharmaceutically acceptable salt thereof. In another embodiment, the Type I SHP2 inhibitor is NSC-87877 or NSC-117199, or a pharmaceutically acceptable salt thereof. In another embodiment, the Type I SHP2 inhibitor is cefsulodin, or a pharmaceutically acceptable salt thereof. In another embodiment, the Type II SHP2 inhibitor is JAB-3068 or JAB-3312, or a pharmaceutically acceptable salt thereof. In another embodiment, the Type II SHP2 inhibitor is RMC-4550 or RMC-4630, or a pharmaceutically acceptable salt thereof. In another embodiment, the Type II SHP2 inhibitor is a SHP099, SHP244, SHP389, SHP394, or TNO155, or a pharmaceutically acceptable salt thereof. In another embodiment, the Type II SHP2 inhibitor is RG-6433 or RLY-1971, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and RMC-4630. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and JAB-3068. In another embodiment, the SHP2 inhibitor is BBP-398, IACS-15509, or IACS-13909, or a pharmaceutically acceptable salt thereof. In another embodiment, the SHP2 inhibitor is X37, or a pharmaceutically acceptable salt thereof. In another embodiment, the SHP2 inhibitor is ERAS-601, or a pharmaceutically acceptable salt thereof. In another embodiment, the SHP2 inhibitor is SH3809, or a pharmaceutically acceptable salt thereof. In another embodiment, the SHP2 inhibitor is HBI-2376, or a pharmaceutically acceptable salt thereof. In another embodiment, the SHP2 inhibitor is ETS-001, or a pharmaceutically acceptable salt thereof. In another embodiment, the SHP2 inhibitor is PCC0208023, or a pharmaceutically acceptable salt thereof. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a use of the compound of Formula I, or a pharmaceutically acceptable salt thereof, administering to a patient in need thereof, a dose between about 50 mg and about 200 mg of the compound of Formula I, or a pharmaceutically acceptable salt thereof, in simultaneous, separate or sequential combination with a second therapeutic agent.

In another aspect, disclosed herein is a use of the compound of Formula I, or a pharmaceutically acceptable salt thereof, in the manufacture of a medicament for treating a KRAS G12C mutant cancer, wherein the compound is administered at a dose between about 50 mg and about 200 mg, in simultaneous, separate or sequential combination with a second therapeutic agent, wherein the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

In another aspect, disclosed herein is a use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with a PD-1 or PD-L1 inhibitor, for treatment of a KRAS G12C mutant cancer. In another embodiment, the PD-1 or PD-L1 inhibitor is pembrolizumab, wherein pembrolizumab is dosed (or administered) 200 mg once every three weeks. In another embodiment, the PD-1 or PD-L1 inhibitor is nivolumab. In another embodiment, the PD-1 or PD-L1 inhibitor is cimiplimab. In another embodiment, the PD-1 or PD-L1 inhibitor is sintilimab. In another embodiment, the PD-1 or PD-L1 inhibitor is atezolizumab. In another embodiment, the PD-1 or PD-L1 inhibitor is avelumab. In another embodiment, the PD-1 or PD-L1 inhibitor is durvalumab. In another embodiment, the PD-1 or PD-L1 inhibitor is lodapilimab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC, wherein pembrolizumab is dosed intravenously at 200 mg once every three weeks. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a use of the compound of Formula I, or a pharmaceutically acceptable salt thereof, in the manufacture of a medicament, administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50% In an embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament, administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50%. In an embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament, administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC. In an embodiment, pembrolizumab is administered for up to thirty-five cycles. In an embodiment, the patient in need thereof is treatment naïve to KRAS G12Ci, PD-1, or PD-L1 therapy. In an embodiment, the patient in need thereof has received at least one treatment, including prior KRAS G12C inhibitor. In yet another embodiment, the patient in need thereof has a tumor proportion score (TPS) status of 0% to 49%. In yet another embodiment, the KRAS G12C-mutant advanced NSCLC has a tumor proportion score (TPS) status of 0% to 49%. In an embodiment, the patient in need thereof has a tumor proportion score (TPS) status of 1% to 49%. In an embodiment, the KRAS G12C-mutant advanced NSCLC has a tumor proportion score (TPS) status of 1% to 49%. In an embodiment, the patient in need thereof has a tumor proportion score (TPS) status of greater than or equal to 50%. In an embodiment, the KRAS G12C-mutant advanced NSCLC has a tumor proportion score (TPS) status of greater than or equal to 50%.

In another aspect, disclosed herein is a use of the compound of Formula I, or a pharmaceutically acceptable salt thereof, in the manufacture of a medicament, administering to a patient in need thereof, a dose of about 150 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 100 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 50 mg of the compound of Formula I, or a pharmaceutically acceptable salt thereof, in simultaneous, separate or sequential combination with pembrolizumab, pemetrexed, and cisplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, pembrolizumab is administered for up to thirty-five cycles. In an embodiment, cisplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 150 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 100 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 50 mg of the compound of Formula I, or a pharmaceutically acceptable salt thereof, in simultaneous, separate or sequential combination with pembrolizumab, pemetrexed, and carboplatin in the treatment of KRAS G12C-mutant advanced NSCLC. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, pembrolizumab is administered for up to thirty-five cycles. In an embodiment, carboplatin is administered for up to four cycles. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 150 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 100 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 50 mg of the compound of Formula I, or a pharmaceutically acceptable salt thereof, in simultaneous, separate or sequential combination with pembrolizumab, pemetrexed, and cisplatin, wherein the combination of the compound of Formula I, or a pharmaceutically acceptable salt thereof, pembrolizumab, pemetrexed, and cisplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, cisplatin is dosed (or administered) 75 mg/m2 once every three weeks. In an embodiment, pemetrexed and cisplatin are administered on the same day. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 150 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 100 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is a use of the compound of Formula I, or a pharmaceutically acceptable salt thereof, in the manufacture of a medicament, administering to a patient in need thereof, a dose of about 50 mg of the compound of Formula I, or a pharmaceutically acceptable salt thereof, in simultaneous, separate or sequential combination with pembrolizumab, pemetrexed, and carboplatin, wherein the combination of the compound of Formula I, or a pharmaceutically acceptable salt thereof, pembrolizumab, pemetrexed, and carboplatin is administered for up to four cycles followed by administering to a patient in need thereof, a dose of about 150 mg of the compound of Formula I, or a pharmaceutically acceptable salt thereof, in simultaneous, separate or sequential combination with pembrolizumab and pemetrexed in the treatment of KRAS G12C-mutant advanced NSCLC, wherein pembrolizumab is administered for up to an additional thirty-one cycles. In an embodiment, pembrolizumab is dosed (or administered) 200 mg once every three weeks. In an embodiment, pemetrexed is dosed (or administered) 500 mg/m2 once every three weeks. In an embodiment, carboplatin is dosed (or administered) area under the curve (AUC) 5 mg/ml/minute once every three weeks for a maximum dose of 750 mg. In an embodiment, pemetrexed and carboplatin are administered on the same day. In an embodiment, the patient in need thereof is treatment naïve to KRAS G12Ci, PD-1, or PD-L1 therapy.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with a CDK4/CDK6 inhibitor, or a pharmaceutically acceptable salt thereof. In another embodiment, the CDK4/CDK6 inhibitor is abemaciclib, wherein abemaciclib is administered 150 mg BID. In another embodiment, the CDK4/CDK6 inhibitor is palbociclib. In another embodiment, the CDK4/CDK6 inhibitor is ribociclib. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with an EGFR inhibitor, or a pharmaceutically acceptable salt thereof. In another embodiment, the EGFR inhibitor is erlotinib, wherein erlotinib is administered 150 mg once a day. In another embodiment, the EGFR inhibitor is afatinib. In another embodiment, the EGFR inhibitor is gefitinib. In another embodiment, the EGFR inhibitor is cetuximab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with cetuximab in the treatment of KRAS G12C-mutant advanced CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with an ERK inhibitor, or a pharmaceutically acceptable salt thereof. In another embodiment, the ERK inhibitor is Temuterkib, wherein Temuterkib is administered 400 mg twice a day. In another embodiment, the ERK inhibitor is LTT462. In another embodiment, the ERK inhibitor is KO-947. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with a platinum agent. In another embodiment, the platinum agent is cisplatin. In another embodiment, the platinum agent is carboplatin. In another embodiment, the platinum agent is oxaliplatin. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, for use in simultaneous, separate or sequential combination with an antifolate, for the treatment of a KRAS G12C mutant cancer. In another embodiment, the antifolate is pemetrexed. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, and an Aurora A inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC. In another embodiment, the Aurora A inhibitor is an aminopyridine compound, or a pharmaceutically acceptable salt thereof. In another embodiment, the Aurora A inhibitor is an Aurora A selective inhibitor, or a pharmaceutically acceptable salt thereof. In another embodiment, the Aurora A inhibitor is alisertib as described in WO 2008/063525. In another embodiment, the Aurora A inhibitor is a pan Aurora inhibitor, or a pharmaceutically acceptable salt thereof. In another embodiment, the Aurora A inhibitor is tozasertib as described in WO 2004/000833. In another embodiment, the Aurora A inhibitor is danusertib as described in WO 2005/005427. In another embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, and the SHP2 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC. In an embodiment, the SHP2 inhibitor is a Type I SHP2 Inhibitor or a Type II SHP2 Inhibitor. In another embodiment, the Type I SHP2 inhibitor is PHPS1 or GS-493, or a pharmaceutically acceptable salt thereof. In another embodiment, the Type I SHP2 inhibitor is NSC-87877 or NSC-117199, or a pharmaceutically acceptable salt thereof. In another embodiment, the Type I SHP2 inhibitor is cefsulodin, or a pharmaceutically acceptable salt thereof. In another embodiment, the Type II SHP2 inhibitor is JAB-3068 or JAB-3312, or a pharmaceutically acceptable salt thereof. In another embodiment, the Type II SHP2 inhibitor is RMC-4550 or RMC-4630, or a pharmaceutically acceptable salt thereof. In another embodiment, the Type II SHP2 inhibitor is a SHP099, SHP244, SHP389, SHP394, or TNO155, or a pharmaceutically acceptable salt thereof. In another embodiment, the Type II SHP2 inhibitor is RG-6433 or RLY-1971, or a pharmaceutically acceptable salt thereof. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and RMC-4630. In another aspect, disclosed herein is a method of treating a KRAS G12C mutant cancer, comprising administering to a patient in need thereof, a dose between about 50 mg and about 200 mg of the compound of Formula I, or a pharmaceutically acceptable salt thereof, and JAB-3068. In another embodiment, the SHP2 inhibitor is BBP-398, IACS-15509, or IACS-13909, or a pharmaceutically acceptable salt thereof. In another embodiment, the SHP2 inhibitor is X37, or a pharmaceutically acceptable salt thereof. In another embodiment, the SHP2 inhibitor is ERAS-601, or a pharmaceutically acceptable salt thereof. In another embodiment, the SHP2 inhibitor is SH3809, or a pharmaceutically acceptable salt thereof. In another embodiment, the SHP2 inhibitor is HBI-2376, or a pharmaceutically acceptable salt thereof. In another embodiment, the SHP2 inhibitor is ETS-001, or a pharmaceutically acceptable salt thereof. In another embodiment, the SHP2 inhibitor is PCC0208023, or a pharmaceutically acceptable salt thereof. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of the compound of Formula I, or a pharmaceutically acceptable salt thereof, in the manufacture of a medicament, administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

### EXAMPLES

### Example 1

### Anti-Tumor Growth Activity in Lung Cancer H358 Xenograft Mouse Model

The purpose of this assay is to determine anti-tumor activity of a compound of Formula I in a lung cancer H358 mouse xenograft model. H358 lung tumor cells (10×10⁶) are implanted by subcutaneous injection in hind leg of nude female mice (Taconic Biosciences). A total of 4 mice in each group are used for the efficacy study. Treatment is initiated with oral administration (gavage) of the test compound or vehicle (20% Captisol®, 25 mM phosphate, pH 2.0 in 0.2 mL volume) once or twice daily for 28 days when the tumor size reaches approximately 300 mg. Tumor growth and body weight are monitored over time to evaluate efficacy and signs of toxicity. The summarized results are provided in Table 1. At 30 mg/kg on a twice daily dosing schedule, −67.68 to −72.14% tumor regression was observed for a compound of Formula I. No significant animal body weight loss was observed through the whole study for this compound.

TABLE 1
Anti-tumor Growth Activity in Lung Cancer H358 Xenograft Mouse Model

| | Dose | Dosing | % Regression | % Regression |
|---|---|---|---|---|
| Treatment | (mg/kg) | Schedule | (Day 12) | (Day 26) |
| a compound of | 30 | PO, BID | −72.14 | −67.68 |
| Formula I | | | | |

### Example 2

### Anti-Tumor Growth Activities in Other Lung Cancer Models and Models of Colorectal, Pancreatic, Bladder and Esophageal Cancers

In addition to H358 xenograft model, a compound of Formula I is tested in other xenograft or patient-derived xenograft (PDX) models of lung, colorectal, pancreatic, bladder, and esophageal cancer at different doses. For xenograft models of H1373, HCC44, MiaPaca-2, SW1463, SW837, KYSE-410 and UM-UC-3, typically 5-10×10⁶ cells in a 1:1 matrigel mix (0.2 mL total volume) are implanted by subcutaneous injection in hind leg of nude mice. Generally, a total of 4 mice each group are used for efficacy study. Treatment is initiated with oral administration (gavage) of testing compound or vehicle (20% captisol, 25 mM phosphate, pH2.0) in 0.2 mL volume when tumor size reaches approximately 200-300 mg. Tumor growth and body weight are monitored over time to evaluate efficacy and signs of toxicity. For EL3187 PDX model, frozen vials containing tumor fragments are thawed at 37° C. in a water bath. The tumor fragments are transferred to 50 mL Falcon tube and the ice cold DMEM medium is slowly added into the tube to a total volume of 35 mL. Then the tumor fragments are centrifuged at 130×g for 2 minutes at 4° C. and supernatant is aspirated. This washing step is repeated twice and the tumor fragments are resuspended in 10 mL DMEM for implantation into athymic nude-Foxn1nu mice (Envigo RMS, Inc., Mount Comfort, Indiana). Once tumor volumes reach 800 to 1000 mm³, animals are sacrificed and tumors are harvested using aseptic technique. Fresh tumors are cut into 10 to 15 mm³ fragments and placed into cold Gibco hibernate medium. The tumor fragments are subcutaneously implanted into animals with a 10 g trochar needle. When tumor size reaches 200 to 300 mm³, the animals are randomized for compound treatment.

The anti-tumor growth or regression activities of a compound of Formula I are summarized in Table 2. Among models listed in Table 2, EL3187 is a patient-derived xenograft (PDX) model, and all other are tumor xenograft models. As illustrated in Table 2, a compound of Formula I demonstrates dose-dependent anti-tumor activities in all of the models, suggesting that a compound of Formula I is active against cancers with KRasG12C mutation, including lung, colorectal, pancreatic, bladder, and esophageal cancer.

TABLE 2
Anti-tumor Growth Activity by a compound of Formula I in Lung, Colorectal,
Pancreatic, Bladder and Esophageal Cancer Xenograft or PDX Models.

| | a compound of Formula I tumor growth inhibition (%) or regression | | | | | | | | | |
|---|---|---|---|---|---|---|---|---|---|---|
| Xenograft/ | (− %) | | | | | | | | | |
| PDX model | 1 mg/kg | | 3 mg/kg | | 5 mg/kg | | 10 mg/kg | | 30 mg/kg | |
| (tumor | QD | BID | QD | BID | QD | BID | QD | BID | QD | BID |
| type) | x28 | x28 | x28 | x28 | x28 | x28 | x28 | x28 | x28 | x28 |
| H1373 | | | | | 97.9 | −26.3 | −21.3 | −49 | −49.7 | |
| (lung) | | | | | | | | | | |
| HCC44 | | | | | 28.3 | 57.5 | 32.7 | 61.8 | 77 | 84.1 |
| (lung) | | | | | | | | | | |
| EL3187 | 52 | 89.2 | −39.8 | −91 | | | −96 | −96 | | |
| (lung) | | | | | | | | | | |
| MiaPaca-2 | | | | | 81.7 | −36.1 | −27 | −45.2 | −42 | −43.6 |
| (pancreas) | | | | | | | | | | |
| SW1463 | | | | | 52.5 | 48.8 | 78.3 | 79.5 | 89.8 | 98.8 |
| (CRC) | | | | | | | | | | |
| SW837 | | | | | 18.2 | 83.6 | 95.2 | 62.1 | 83.5 | −23.8 |
| (CRC) | | | | | | | | | | |
| KYSE-410 | | | | | | | 25.3 | 51.5 | 66.7 | 61.8 |
| (Esophagus) | | | | | | | | | | |
| UM-UC-3 | | | | | 34.3 | 45.3 | 33.6 | 75 | 83.1 | 85 |
| (bladder) | | | | | | | | | | |

### Example 3

### Study of a Compound of Formula I in Patients with KRAS G12C-Mutant Advanced Solid Tumors

Study, NCT04956640, is a first-in-human, multicenter, open-label Phase 1a/1b study to evaluate the safety, tolerability, and preliminary efficacy of orally administered compound of Formula I. The compound of Formula I is being evaluated as monotherapy and as part of combination therapy in patients with KRAS G12C-mutant advanced solid tumor types including, but not limited to, NSCLC and CRC.

NCT04956640 includes Phase 1a dose escalation (Part A of NCT04956640, see Table 3) followed by a Phase 1b dose expansion (Parts B to E of NCT04956640, see Table 4). The Phase 1a dose escalation compound of Formula I monotherapy cohorts (See Table 3) will enroll any eligible patient with KRAS G12C-mutant advanced solid tumor. Phase 1b dose expansion will include 12 cohorts (See Table 4) to further evaluate safety and clinical activity.

KRAS G12C mutations will be identified through standard-of-care testing as routinely performed at each participating site utilizing material collected prior to patient consent to this protocol. Molecular assays utilized for enrollment are required to be performed in Clinical Laboratory Improvement Amendments (CLIA), International Organization for Standardization/International Electrotechnical Commission (ISO/IEC), College of American Pathologists (CAP), or similarly certified laboratory.

TABLE 3
NCT04956640 Phase 1a dose escalation

| | Study Drug | | | |
|---|---|---|---|---|
| | (Route of | | Dose | Dose |
| | Administration) | Cohort | Amount^{a} | Frequencyª |
| Monotherapy/ | a compound of | A1 | 50 mg | BID |
| Part A | Formula I (PO) | A2 | 100 mg | BID |
| | | A3 | 150 mg | BID |
| | | A4 | 200 mg^{b} | BID |

Abbreviations: BID = twice daily; DL = dose level; PO = orally; PK = pharmacokinetics; QD = once daily; SRC = Safety Review Committee
^{a}Lower or intermediate DLs, as well as alternative dosing schedules (i.e., QD), may be considered by SRC based upon totality of safety, PK/pharmacodynamics, and efficacy data. Higher dosing levels may be supported by clinical safety determined at lower dosing levels.

TABLE 4
NCT04956640 Phase 1b dose expansion

| | | Study Drug(s) | Dose Amount^{a} |
|---|---|---|---|
| | Cohort | (Route of Administration) | (Dosing Frequency) |
| Part B: | B1 | a compound of Formula I, or | 50-200 mg (BID) [e.g., 50 |
| KRAS G12C- | | pharmaceutically acceptable | mg, 100 mg, 150 mg, or |
| mutant | | salt thereof (PO) | 200 mg (BID)] |
| advanced | B2 | a compound of Formula I, or | 50-200 mg (BID) [e.g., 50 |
| NSCLC | | pharmaceutically acceptable | mg, 100 mg, 150 mg, or |
| | | salt thereof (PO) | 200 mg (BID)] |
| | | LY2835219 (abemaciclib) (PO) | 150 mg (BID) |
| | B3 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | Erlotinib (PO) | 150 mg (QD) |
| | B4 | a compound of Formula I, or | 50-200 mg (BID) [E.G., |
| | | pharmaceutically acceptable | 50 mg, 100 mg, 150 mg, |
| | | salt thereof (PO) | OR 200 mg (BID)] |
| | | Pembrolizumab (IV) | 200 mg (Q3W) |
| | B5 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | Temuterkib (ERK inhibitor) | 400 mg (QD) |
| | | (PO) | |
| | B6 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | LY3295668 | 25 mg (BID) |
| | | (Aurora A kinase inhibitor) | |
| | | (PO) | |
| | B7 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | TNO155 | Dose escalation: 20-60 mg |
| | | (SHP2 inhibitor) (PO) | (QD) [e.g., 20 mg, 40 mg, |
| | | | or 60 mg (QD)] (QD; 2 |
| | | | weeks on, 1 week off) |
| | B8 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | B9 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | Pembrolizumab (IV) | 200 mg (Q3W) |
| | | Pemetrexed (IV) | 500 mg/m2 (Q3W) |
| | | Cisplatin (IV) | 75 mg/m2 (Q3W) |
| | | OR | OR |
| | | Carboplatin (IV) | AUC 5 mg/ml/min (Q3W) |
| | | | (max dose = 750 mg) |
| Part C: | C1 | a compound of Formula I, or | 50-200 mg (BID) [e.g., 50 |
| KRAS G12C- | | pharmaceutically acceptable | mg, 100 mg, 150 mg, or |
| mutant | | salt thereof (PO) | 200 mg (BID)] |
| advanced | | Temuterkib (ERK inhibitor) | 400 mg (QD) |
| CRC | | (PO) | |
| | C2 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | Cetuximab (IV) | 400 mg/m² loading dose |
| | | | C1D1; |
| | | | 250 mg/m² every week |
| | | | thereafter (QW) |
| | C3 | a compound of Formula I, or | 50-200 mg (BID) [e.g., 50 |
| | | pharmaceutically acceptable | mg, 100 mg, 150 mg, or |
| | | salt thereof (PO) | 200 mg (BID)] |
| | | TNO155 | Dose escalation: 20-60 mg |
| | | (SHP2 inhibitor) (PO) | (QD) [e.g., 20 mg, 40 mg, |
| | | | or 60 mg (QD)] (QD; 2 |
| | | | weeks on, 1 week off) |
| Part D: | D1 | a compound of Formula I, or | 50-200 mg (BID) [e.g., 50 |
| KRAS G12C- | | pharmaceutically acceptable | mg, 100 mg, 150 mg, or |
| mutant | | salt thereof (PO) | 200 mg (BID)] |
| advanced solid | | | |
| tumors (except | | | |
| NSCLC and | | | |
| CRC) | | | |
| Part E: | E1 | a compound of Formula I, or | 50-200 mg (BID) [e.g., 50 |
| KRAS G12C- | | pharmaceutically acceptable | mg, 100 mg, 150 mg, or |
| mutant | | salt thereof (PO) | 200 mg (BID)] |
| advanced | | | |
| NSCLC who | | | |
| have previously | | | |
| been treated | | | |
| with a KRAS | | | |
| G12C inhibitor | | | |
| Part F: | F1 | a compound of Formula I, or | 50-200 mg (BID) [e.g., 50 |
| KRAS G12C- | | pharmaceutically acceptable | mg, 100 mg, 150 mg, or |
| mutant | | salt thereof (PO) | 200 mg (BID)] |
| advanced | | | |
| pancreatic | | | |
| cancer | | | |
| Part G: | G1 | a compound of Formula I, or | 50-150 mg (BID) [e.g., 50 |
| Previously | | pharmaceutically acceptable | mg, 100 mg, or 150 mg |
| untreated, PD- | | salt thereof (PO) | (BID)] |
| L1-positive | | Pembrolizumab (IV) | 200 mg (Q3W) |
| KRAS G12C- | | | |
| mutant | G2 | a compound of Formula I, or | 50-150 mg (BID) [e.g., 50 |
| advanced | | pharmaceutically acceptable | mg, 100 mg, or 150 mg |
| NSCLC | | salt thereof (PO) | (BID)] |
| | | Pembrolizumab (IV) | 200 mg (Q3W) |

Abbreviations: BID = twice daily; CRC = colorectal cancer; ERK = extracellular signal regulated kinase; IV = intravenous; NSCLC = non-small cell lung cancer; PO = orally; QD = once daily; QW = every week; Q3W = every 3 weeks.
^{a}Lower or intermediate DLs, as well as alternative dosing schedules (i.e., QD), may be considered by SRC based upon totality of safety, PK/pharmacodynamics, and efficacy data. Higher dosing levels may be supported by clinical safety determined at lower dosing levels.

Eligibility Criteria:

Inclusion Criteria

Each patient must meet all of the following inclusion criteria to be eligible to participate in the NCT04956640 study:
1. Each patient must be ≥18 years of age at the time of signing the informed consent.
2. Patients are eligible if they have measurable disease per Response Evaluation Criteria in Solid Tumors version 1.1 (RECIST v1.1).
3. Patients must have disease with evidence of KRAS G12C mutation in tumor tissue or circulating tumor deoxyribonucleic acid (DNA) as determined by molecular testing routinely performed at a CLIA, ISO/IEC, College of American Pathologists (CAP), or other similar certified laboratory. For dose escalation backfill cohorts and Cohort E1, NSCLC patients who have progressed on a prior KRAS G12C inhibitor must have KRAS G12C mutation confirmed in a blood or tumor tissue sample collected after discontinuation of prior KRAS G12C inhibitor.
4. Each patient must have a histological or a cytologically proven diagnosis of locally advanced, unresectable, and/or metastatic cancer and meet cohort-specific criteria:
   a) Phase 1a Dose Escalation:
      Patients must be appropriate candidates for experimental therapy, where palliative measures are no longer effective or are not considered appropriate or safe in the opinion of an Investigator.
   b) Phase 1b Dose Expansion Cohorts B (NSCLC):
      Patients must not have a known targetable oncogenic driver mutation or alteration in genes such as EGFR, ALK, BRAF (V600E), MET (exon 14), ROS1, RET, or NTRK1/2/3.
      Patients must be appropriate candidates for experimental therapy, after progressing on, being intolerant to, or ineligible for immunotherapy and platinum-based therapy, and where palliative measures are no longer effective or are not considered appropriate or safe in the opinion of the Investigator. If the available standard therapy is not considered appropriate or safe in the opinion of the Investigator, the rationale for ineligibility shall be provided and documented in the CRF. Furthermore, in the opinion of the Investigator, if the patient is intolerant to standard therapy, drug information, toxicity, and grade must be documented in the CRF.
      Cohort B9 Only Specific Criteria
      Histologically or cytologically confirmed Stage IIIB-IIIC or Stage IV NSCLC that is previously untreated in the advanced/metastatic setting and not suitable for curative intent radical surgery or radiation therapy. Staging will be according to the American Joint Committee on Cancer (AJCC) Staging System (8th ed). The histology of the tumor must be predominantly nonsquamous. Squamous cell and/or mixed small cell/non-small cell histology is not permitted.
      Patients may have received up to one 21-day cycle of pembrolizumab plus pemetrexed with either cisplatin or carboplatin at the dose levels defined for Cohort B9 in Table 4. Such therapy must have been initiated within 21 days (+14 days) prior to enrollment.
      Cohort B8 Only Specific Criteria
      Patients must have at least 1 untreated, active brain metastasis, defined as a new and/or growing lesion at least 5 mm in diameter, without any subsequent local treatment before the start of study treatment, and without associated neurological symptoms requiring urgent intervention with locoregional therapy. Patients with leptomeningeal disease are not eligible. Prophylactic anticonvulsants are permitted, provided the participant is on a stable dose for ≥14 days prior to Cycle 1 Day 1 (C1D1), except those taking enzyme-inducing anti-epileptic drugs must have discontinued such therapy with a washout period equivalent to 5 half-lives of the drug.
      Remaining Part B Cohorts
      Patients must be appropriate candidates for study treatment, after progressing on, being intolerant to, or ineligible for immunotherapy and platinum-based therapy. Progressing on, being intolerant to, or ineligible for immunotherapy is NOT applicable to patients in Cohort B4.
   c) Phase 1b Dose Expansion Cohorts C (CRC):
      Patients must be appropriate candidates for experimental therapy, and must have received cytotoxic chemotherapy containing fluoropyrimidine, oxaliplatin, and irinotecan for advanced or metastatic CRC, and where palliative measures are no longer effective or are not considered appropriate or safe in the opinion of the Investigator.
      For Cohort C2, patients are only be enrolled if, in the Investigator's judgment, the patient is appropriate to initiate cetuximab at the standard dose for cetuximab in the cetuximab package insert.
   d) Phase 1b Dose Expansion Cohort D (solid tumors except NSCLC and CRC):
      Patients must have histological or cytologically proven recurrent/metastatic, unresectable solid tumors except NSCLC and CRC, and with KRAS G12C mutation.
      Patients must be appropriate candidates for experimental therapy, where palliative measures are no longer effective or are not considered appropriate or safe in the opinion of the Investigator.
   e) Phase 1b Dose Expansion Cohort E (NSCLC):
      Patients must have progressed on a KRAS G12C inhibitor.
      Patients must be appropriate candidates for experimental therapy, where palliative measures are no longer effective or are not considered appropriate or safe in the opinion of the Investigator.
   f) Phase 1b Dose Expansion Cohort F (pancreatic cancer):
      Patients must have histological or cytologically proven recurrent/metastatic, unresectable pancreatic cancer with KRAS G12C mutation.
      Patients must be appropriate candidates for experimental therapy, where palliative measures are no longer effective or are not considered appropriate or safe in the opinion of the Investigator.
   g) Phase 1b Dose Optimization Part G (NSCLC)
      Patients must not have additional validated oncogenic drivers in NSCLC, if known, e.g., activating alterations in genes such as EGFR, ALK, BRAF (V600E), MET (exon 14), ROS1, RET, or NTRK1/2/3.
      Patients must have histologically or cytologically confirmed Stage IIIB-IIIC or Stage IV NSCLC, previously untreated in the advanced/metastatic setting and not suitable for curative intent radical surgery or radiation therapy. Staging will be according to the AJCC Staging System (8th ed).
      Patient may have received up to one previous cycle of pembrolizumab prior to administering to a patient in need thereof, a dose of the compound of Formula I, or a pharmaceutically acceptable salt thereof. The one previous cycle of pembrolizumab must have been administered no more than 35 days prior to administering to a patient in need thereof, a dose of the compound of Formula I, or a pharmaceutically acceptable salt thereof.
      Patients may have a PD-L1-positive (TPS ≥1%) tumor as determined by IHC using anti-PD-L1 antibody clone 22C3 at a local or sponsor-designated laboratory with CLIA, ISO/IEC, CAP, or other similar certification as per local guidelines including, but not limited to, IVDR compliance as applicable.
5. Have an Eastern Cooperative Oncology Group (ECOG) Performance Status of 0 or 1 (See Oken M M, Creech R H, Tormey D C, et al. Toxicity and response criteria of the Eastern Cooperative Oncology Group. Am J Clin Oncol. 1982; 5(6):649-655).
6. Have adequate organ function, as defined in Table 5 below:

TABLE 5

| | |
|---|---|
| System | Laboratory Value |

Hematologic

| | |
|---|---|
| ANC | ≥1.5 × 10⁹/L |
| Platelets | ≥100 × 10⁹/L |
| Hemoglobin | ≥ 9 g/dL |

Hepatic

| | |
|---|---|
| TBL | ≤1.5 × ULN, |
| | Patients with Gilber's syndrome with a TBL |
| | ≤3.0 times ULN and direct bilirubin within |
| | normal limits are permitted. |
| ALT and AST | ≤2.5 × ULN or |
| | ≤5 × ULN if the liver has tumor involvement. |

Renal

| | |
|---|---|
| Serum creatinine or | <1.5 × ULN or |
| Measured creatinine | ≥50 mL/min (measured OR calculated) |
| clearance or Calculated | |
| creatinine clearance | |

Abbreviations: ALT = alanine aminotransferase; ANC = absolute neutrophil count; AST = aspartate aminotransferase; G-CSF = granulocyte colony-stimulating factor; TBL = total bilirubin; ULN = upper limit of normal.
Note:
Transfusions to increase a patient's hemoglobin level or initiation of erythropoietin or G-CSF therapy to meet enrollment criteria are not allowed in the 14 days prior to enrollment. If a patient receives transfusions, erythropoietin, or G-CSF therapy ≥14 days prior to enrollment, the hematologic criteria listed above must be met following the 14-day window and prior to enrollment.

7. Each patient must have discontinued all previous treatments for cancer with resolution of any AEs, with the exception of alopecia and Grade 2 neuropathy, and of all clinically significant toxic effects of prior locoregional therapy, surgery, radiotherapy, or systemic anticancer therapy to Grade ≤1 or prior baseline. Patients with ongoing endocrinopathies due to prior treatment that have not resolved but are on appropriate replacement therapy (e.g., thyroid, adrenal or pituitary, or pancreatic) are permitted to enroll.
   For Cohort B9 only: Participants with therapy-related toxicities that have developed and are related to a single prior cycle of pembrolizumab plus pemetrexed with carboplatin or cisplatin therapy (with the exception of the immune-related toxicities outlined in Exclusion Criterion #24) may enroll, if the Investigator determines these toxicities are not required to be resolved at the time of study entry and the participant is still an appropriate candidate for study treatment.
8. Each patient must have discontinued from previous treatments, as defined in Table 6 below:

TABLE 6

| | Length of Time |
|---|---|
| | Prior to Start of |
| Previous Treatment | Study Drug(s) |
| Cytotoxic therapies or targeted agents that are | ≥14 days or |
| small molecule inhibitors | ≥5 half-lives, |
| | whichever is shorter |
| Biologic agents that are large molecules including | ≥21 days |
| immunotherapy | |
| Radiotherapy | |
| Limited-field radiotherapy with palliative intent | ≥7 days |
| Other radiotherapy | ≥21 days |
| Major surgery | ≥28 days |

9. Each patient must be able to swallow capsules.
10. Each patient must agree and adhere to contraceptive use by men or women that is consistent with local regulations regarding the methods of contraception for those participating in clinical studies.
11. Each patient that is a woman of childbearing potential (WOCBP) must have a negative serum or urine pregnancy test documented within 7 days prior to enrollment.
12. Each patient must have an estimated life expectancy of ≥12 weeks.
13. Each patient is reliable and is willing to make themselves available for the duration of the study and is willing to follow study procedures.
14. Each patient must be capable of giving signed informed consent.

Exclusion Criteria

Each patient who meets any of the following criteria will be excluded from the LOXO-RAS-20001 study:
1. Disease suitable for local therapy administered with curative intent.
2. Have an active fungal, bacterial, and/or active untreated viral infection, including human immunodeficiency virus (HIV) or viral (A, B, or C) hepatitis.
   Note: For Cohort B4, Cohort B9, and Part G only:
   a) HIV-infected patients must be on anti-retroviral therapy (ART) and have a well-controlled HIV infection/disease defined as:
      Each patient on ART must have a CD4+ T-cell count >350 cells/mm³ at time of screening.
      Each patient on ART must have achieved and maintained virologic suppression defined as confirmed HIV RNA level below 50 copies/mL or the lower limit of qualification (below the limit of detection) using the locally available assay at the time of screening and for at least 12 weeks prior to screening.
      Each patient on ART must have been on a stable regimen, without changes in drugs or dose modification, for at least 4 weeks prior to study entry (Day 1).
      Each HIV-infected patient with a history of Kaposi sarcoma and/or Multicentric Castleman Disease are excluded.
   b) Each patient who are HBsAg positive are eligible if they have received HBV antiviral therapy for at least 4 weeks and have undetectable HBV viral load prior to randomization.
   Note: Each patient should remain on anti-viral therapy throughout study intervention and follow local guidelines for HBV anti-viral therapy post completion of study intervention.
   Hepatitis B screening tests are not required unless:
      Known history of HBV infection
      As mandated by local health authority
   c) Each patient with history of HCV infection are eligible if HCV viral load is undetectable at screening.
   Note: Each patient must have completed curative anti-viral therapy at least 4 weeks prior to randomization.
   Hepatitis C screening tests are not required unless:
      Known history of HCV infection
      As mandated by local health authority
3. The patient has a serious pre-existing medical condition(s) that, in the judgment of the Investigator, would preclude participation in this study, including interstitial lung disease (ILD), severe dyspnea at rest, or requiring oxygen therapy.
4. The patient has clinically significant, active cardiovascular disease, unstable angina, or history of myocardial infarction within 6 months prior to planned start of a compound of Formula I, or QT interval corrected for heart rate (QTcF) of ≥470 msec on screening electrocardiogram (ECG) as calculated using Fridericia's formula (QTcF) at several consecutive days of assessment.
5. Patient has a second active primary malignancy or has been diagnosed and/or treated for an additional malignancy within 3 years prior to enrollment with the exception of curatively-treated basal cell carcinoma of the skin, nonmetastatic prostate cancer treated with observation only, squamous cell carcinoma of the skin, and/or curatively resected in situ cervical and/or breast cancers.
6. For all patients except those in Cohort B8: Patient has a symptomatic central nervous system (CNS) malignancy or metastasis or carcinomatous meningitis. Patients with treated CNS metastases are eligible for this study if they are not currently receiving corticosteroids in excess of 10 mg/day prednisone/prednisolone (or equivalent) and their disease is asymptomatic and radiographically stable for at least 30 days and patients have completed prior CNS-directed therapy (including radiation and/or surgery) ≥28 days prior to the first dose of study intervention, and symptomatically and radiologically stable disease (i.e., without evidence of progression for ≥28 days by repeat imaging). Clinically stable without requirement of steroid treatment within 14 days prior to randomization. Prophylactic anticonvulsants are permitted, provided the patient is on a stable dose for ≥14 days prior to C1D1, except participants taking enzyme-inducing anti-epileptic drugs must have discontinued such therapy with a washout period equivalent to 5 half-lives of the drug.

Prior/Concomitant Therapy
7. Each patient must not have received prior treatment with any KRAS G12C small molecule inhibitor, excepting the following scenarios where such prior therapy is allowed:
   a) Phase 1a dose escalation backfill cohort (NSCLC only)
   b) Cohort E1
   c) Cohort B4
8. Each patient having been treated with drugs known to be strong inhibitors or inducers of cytochrome P450 (CYP)3A (e.g., ketoconazole and rifampin) are not eligible to be enrolled in Cohorts B2, B3, B5/C1, except if the strong inhibitors or inducers of CYP3A treatment has been discontinued (or switched to a different medication) and a wash-out of at least 5 half-lives is implemented prior to starting study drug. Patients treated with drugs carrying risk for QTc prolongation are not eligible to be enrolled in Cohorts B7 and C3.
9. For patients who have received prior immunotherapy, the following patients will be excluded from Cohort B4, Cohort B9, and Part G:
   a) Experienced a Grade 3 immune-related toxicity or any immune-related toxicity that led to permanent discontinuation of prior anti-PD-1, anti-PD-L1, or other immunotherapy
   b) Experienced a 5 Grade 3 immune-related AE (irAE) that has not recovered to Grade 1 after use of corticosteroids that occurred during prior immunotherapy with the exception that endocrine disorders where patients with prior endocrine AEs are permitted to enroll if they are asymptomatic, considered clinically stable, and maintained on appropriate replacement therapy.
   c) Any grade:
      i. Ocular irAE,
      ii. Serious neurologic irAE (e.g., Guillain-Barre syndrome, myasthenia gravis, encephalitis), or
      iii. Serious cardiovascular irAE (e.g., myocarditis).
   d) Required immunosuppressive agents other than corticosteroids for the management of irAE or currently requires maintenance doses of ≥10 mg prednisone/prednisolone (or equivalent) per day for irAE.
10. Each patient will be excluded from Cohort B4, Cohort B9, and Part G with any of the following:
   a) Has an active autoimmune disease that has required systemic antiautoimmune treatment in the past 2 years (i.e., with use of disease modifying agents, corticosteroids, or immunosuppressive drugs). Replacement therapy (e.g., thyroxine, insulin, or physiologic corticosteroid replacement therapy for adrenal or pituitary insufficiency) is not considered a form of systemic treatment and is allowed.
   b).
   c) Has received a live vaccine within 30 days prior to the first dose of study drug. Examples of live vaccines include, but are not limited to, the following: measles, mumps, rubella, varicella/zoster (chicken pox), yellow fever, rabies, Bacillus Calmette-Guerin (BCG), and typhoid vaccine. Seasonal influenza vaccines for injection are generally killed virus vaccines and are allowed; however, intranasal influenza vaccines are live attenuated vaccines and therefore are not allowed.
   d) Has had an allogeneic tissue/solid organ transplant.
   e) Has severe hypersensitivity (≥Grade 3) to pembrolizumab and/or any of its excipients.
   f) Has received radiation therapy to the lung that is >30 Gy within 6 months of the first dose of a compound of Formula I.
      Note: For Cohort B9 and Part G only:
   g) Patient received prior systemic therapy (chemotherapy, immunotherapy, or biological therapy) for advanced or metastatic disease, except as allowed for participants in Cohort B9 in Inclusion Criterion #4. Patient who received adjuvant or neoadjuvant therapy are eligible if the last dose of the systemic treatment was completed at least 6 months prior to enrollment.
      For patients who received immunotherapy in either the neoadjuvant or adjuvant settings, relapse/recurrence of metastatic disease should have occurred at least 6 months after last dose.
      Note: For Cohort B9 only
   h) Patient is unable to interrupt nonsteroidal anti-inflammatory drugs (NSAIDs) 2 days before (5 days for long-acting NSAIDs), the day of, and 2 days following administration of pemetrexed.
   i) Patient is unable or unwilling to take folic acid, dexamethasone, or vitamin B12 supplementation.
j) Patient has a known hypersensitivity to any of the excipients of carboplatin, cisplatin, or pemetrexed.

Other Exclusions
11. Each patient that is pregnant, breastfeeding, or expecting to conceive or father children within the projected duration of the trial, starting with the screening visit through 180 days after the last dose of study medication.
12. Each patient that has a known allergic reaction against any of the components of the study treatments.

For Cohort B4, Cohort B9, and Part G only, patients with a history of (non-infectious) pneumonitis/interstitial lung disease that required steroids or has current pneumonitis/interstitial lung disease.

Patient has a prior enrollment in another cohort in this study.

Table 7 illustrates preliminary NCT04956640 Phase 1a clinical pharmacokinetics data.

TABLE 7

| | 50 mg BID | 100 mg BID | 150 mg BID | 200 mg BID |
|---|---|---|---|---|
| | N = 12 | N = 15 | N = 17 | N = 6 |
| Cmax ng/ml | 144 (45%) | 305 (75%) | 368 (51%) | 325 (61%) |
| CL/F L/h | 79 (53%) | 67.0 (77%) | 83.6 (47%) | 145 (59%) |
| AUC | 634 (53%) | 1475 (77%) | 1795 (47%) | 1372 (59%) |
| ng.h/mL | | | | |
| t½ h | 3.03 (41%)^{c} | 3.01 (33%)^{b} | 3.41 (32%) | 2.35 (58%) |
| Ctrough- | 23.8 (90%)^{c} | 44.9 (73%) | 54.5 (60%) | 40.3 (73%) |
| Cmin ng/mL | | >TEC90 | >TEC90 | >TEC90 |
| | | 25 ng/ml | 25 ng/ml | 25 ng/ml |

N is the number of patients.
^{b}is 14 patients.
^{c}is 11 patients.

200 mg BID has been supported by NCT04956640 Phase 1a clinical safety determined at lower dosing levels. 200 mg BID is supported by clearing DLT at 150 mg BID. 150 mg BID has been supported by clearing DLT at 100 mg BID. 100 mg BID has been supported by clearing DLT at 50 mg BID. Higher dosing levels may be supported by clinical safety determined at lower dosing levels.

Table 8 illustrates preliminary NCT04956640 Phase 1a clinical response data including intrapatient dose escalation.

TABLE 8

| Starting dose | | ORR/ | SD (by | | PD (by |
|---|---|---|---|---|---|
| level | N | DCR | indication) | PR (by indication) | indication) |
| Dose level 1: | 12 | 0%/75% | 9 (3 CRC, 1 | 0 | 3 (1 NSCLC |
| 50 mg BID | | | NSCLC prior | | naïve to |
| | | | KRAS G12Ci, 1 | | KRAS |
| | | | PANC, 4 other) | | G12Ci, 2 |
| | | | | | NSCLC prior |
| | | | | | KRAS |
| | | | | | G12Ci) |
| Dose level 2: | 10 | 40%/80% | 4 (3 CRC, 1 | 4 (1 CRC, 1 | 2 (2 PANC) |
| 100 mg BID | | | other tumor | NSCLC prior | |
| | | | type) | KRAS G12Ci, 1 | |
| | | | | PANC, 1 other | |
| | | | | tumor type) | |
| Dose level 3: | 12⁺ | 18.2%/90.9% | 8 (3 CRC, 1 | 2 (1 PANC, 1 | 1 (1 NSCLC |
| 150 mg BID | | | NSCLC naïve to | other tumor type) | prior KRAS |
| | | | KRAS G12Ci, 2 | | G12Ci) |
| | | | NSCLC prior | | |
| | | | KRAS G12Ci, 1 | | |
| | | | PANC, 1 other | | |
| | | | tumor type) | | |
| Dose level 4: | 7* | 20%/100% | 4 (4 CRC) | 1 (1 other tumor | |
| 200 mg BID | | | | type) | |

⁺11 patients with scan data. *5 patients with scan data.

Table 9 illustrates preliminary NCT04956640 Phase 1a clinical response data by response.

TABLE 9

| | | NSCLC naïve | NSCLC with | | |
|---|---|---|---|---|---|
| Best observed | | to KRAS | prior KRAS | | other tumor |
| response | CRC | G12Ci | G12Ci | PANC | types |
| SD (N = 24) | 13 | 1 | 3 | 2 | 6 |
| (63.2%) | | | | | |
| PR (includes | 1 | | 1 | 2 | 3 |
| both | | | | | |
| unconfirmed | | | | | |
| and | | | | | |
| confirmed) | | | | | |
| (N = 7) | | | | | |
| (18.4%) | | | | | |
| PD (includes | 1 | 1 | 3 | 2 | |
| both | | | | | |
| unconfirmed | | | | | |
| and | | | | | |
| confirmed) | | | | | |
| (N = 7) | | | | | |
| (18.4%) | | | | | |

Tables 10, 11, and 12 illustrate preliminary NCT04956640 Phase 1a, Phase 1b Cohort B4, and Phase 1b Cohort C2 data as of January 2023, respectively, of Treatment-Emergent Adverse Events by Starting Dose of a compound of Formula L, or pharmaceutically acceptable salt thereof.

Tables 10A and 11A illustrate preliminary NCT04956640 Phase 1a and Phase 1b Cohort B4 data as of April 2023, respectively, of Treatment-Emergent Adverse Events by Starting Dose of a compound of Formula L, or pharmaceutically acceptable salt thereof.

TABLE 10
Preliminary NCT04956640 Phase 1a data of Treatment-Emergent
Adverse Events by Starting Dose of a compound of Formula I, or pharmaceutically
acceptable salt thereof

| | Total | 50 mg BID | 100 mg BID | 150 mg BID | 200 mg BID |
|---|---|---|---|---|---|
| | (N = 84) | (N = 15) | (N = 29) | (N = 27) | (N = 13) |
| Patients (Pts) with | 80 | 14 | 27 | 26 | 13 |
| TEAEs, n (%) | (95.2) | (93.3) | (93.1) | (96.3) | (100.0) |
| Pts with TEAEs | 58 | 6 | 19 | 21 | 12 |
| Related to a | (69.0) | (40.0) | (65.5) | (77.8) | (92.3) |
| compound of | | | | | |
| Formula I, n (%) | | | | | |
| Pts with TEAEs | 28 | 6 | 9 | 9 | 4 |
| Maximum Severity 3 | (33.3) | (40.0) | (31.0) | (33.3) | (30.8) |
| or 4, n (%) | | | | | |
| Pts with TEAEs | 4 | 0 | 0 | 2 | 2 |
| Maximum Severity 3 | (4.8) | (0.0) | (0.0) | (7.4) | (15.4) |
| or 4 and Related to a | | | | | |
| compound of | | | | | |
| Formula I, n (%) | | | | | |
| Pts with Serious | 17 | 5 | 8 | 3 | 1 |
| TEAEs, n (%) | (20.2) | (33.3) | (27.6) | (11.1) | (7.7) |
| Pts with Serious | 0 | 0 | 0 | 0 | 0 |
| TEAEs and Related | (0.0) | (0.0) | (0.0) | (0.0) | (0.0) |
| to a compound of | | | | | |
| Formula I, n (%) | | | | | |
| Pts with Fatal | 2 | 1 | 1 | 0 | 0 |
| TEAEs, n (%) | (2.4) | (6.7) | (3.4) | (0.0) | (0.0) |
| Pts with Fatal | 0 | 0 | 0 | 0 | 0 |
| TEAEs and Related | (0.0) | (0.0) | (0.0) | (0.0) | (0.0) |
| to a compound of | | | | | |
| Formula I, n (%) | | | | | |

As can be seen in Table 10 above, Phase 1a patients did not experience a serious TEAE related to a compound of Formula I, or pharmaceutically acceptable salt thereof. As can be seen in Table 10 above, Phase 1a patients did not experience a fatal TEAE related to a compound of Formula I, or pharmaceutically acceptable salt thereof.

TABLE 10A
Preliminary NCT04956640 Phase 1a data of Treatment-Emergent
Adverse Events by Starting Dose of a compound of Formula I, or pharmaceutically
acceptable salt thereof

| | Total | 50 mg BID | 100 mg BID | 150 mg BID | 200 mg BID |
|---|---|---|---|---|---|
| | (N = 109) | (N = 16) | (N = 32) | (N = 48) | (N = 13) |
| Patients (Pts) with | 99 | 16 | 29 | 41 | 13 |
| TEAEs, n (%) | (90.8) | (100) | (90.6) | (85.4) | (100.0) |
| Pts with TEAEs | 67 | 8 | 19 | 28 | 12 |
| Related to a | (61.5) | (50.0) | (59.4) | (58.3) | (92.3) |
| compound of | | | | | |
| Formula I, n (%) | | | | | |
| Pts with TEAEs | 38 | 7 | 14 | 14 | 3 |
| Maximum Severity | (34.9) | (43.8) | (43.8) | (29.2) | (23.1) |
| 3 or 4, n (%) | | | | | |
| Pts with TEAEs | 6 | 0 | 0 | 5 | 1 |
| Maximum Severity | (5.5) | (0.0) | (0.0) | (10.4) | (7.7) |
| 3 or 4 and Related to | | | | | |
| a compound of | | | | | |
| Formula I, n (%) | | | | | |
| Pts with Serious | 25 | 6 | 13 | 5 | 1 |
| TEAEs, n (%) | (22.9) | (37.5) | (40.6) | (10.4) | (7.7) |
| Pts with Serious | 1 | 0 | 0 | 1 | 0 |
| TEAEs and Related | (0.9) | (0.0) | (0.0) | (2.1) | (0.0) |
| to a compound of | | | | | |
| Formula I, n (%) | | | | | |
| Pts with Fatal | 2 | 1 | 1 | 0 | 0 |
| TEAEs, n (%) | (1.8) | (6.3) | (3.1) | (0.0) | (0.0) |
| Pts with Fatal | 0 | 0 | 0 | 0 | 0 |
| TEAEs and Related | (0.0) | (0.0) | (0.0) | (0.0) | (0.0) |
| to a compound of | | | | | |
| Formula I, n (%) | | | | | |

TABLE 11
Preliminary NCT04956640 Phase 1b Cohort B4 data of Treatment-
Emergent Adverse Events by Starting Dose of a compound of Formula I, or
pharmaceutically acceptable salt thereof

| | Total | 50 mg BID | 100 mg BID | 150 mg BID |
|---|---|---|---|---|
| | (N = 16) | (N = 4) | (N = 6) | (N = 6) |
| Patients (Pts) with TEAEs, n (%) | 13 | 2 | 5 | 6 |
| | (81.3) | (50.0) | (83.3) | (100.0) |
| Pts with TEAEs Related to a | 11 | 2 | 3 | 6 |
| compound of Formula I, n (%) | (68.8) | (50.0) | (50.0) | (100.0) |
| Pts with TEAEs Related to | 11 | 2 | 3 | 6 |
| Pembrolizumab, n (%) | (68.8) | (50.0) | (50.0) | (100.0) |
| Pts with TEAEs Maximum | 6 | 1 | 1 | 4 |
| Severity 3 or 4, n (%) | (37.5) | (25.0) | (16.7) | (66.7) |
| Pts with TEAEs Maximum | 4 | 1 | 1 | 2 |
| Severity 3 or 4 and Related to a | (25.0) | (25.0) | (16.7) | (33.3) |
| compound of Formula I, n (%) | | | | |
| Pts with TEAEs Maximum | 3 | 1 | 0 | 2 |
| Severity 3 or 4 and Related to | (18.8) | (25.0) | (0.0) | (33.3) |
| Pembrolizumab, n (%) | | | | |
| Pts with a compound of Formula | 4 | 1 | 1 | 2 |
| I,, or pharmaceutically | (25.0) | (25.0) | (16.7) | (33.3) |
| acceptable salt thereof and/or | | | | |
| Pembrolizumab related grade | | | | |
| >=3 TEAEs, n (%) | | | | |
| Pts with Serious TEAEs, n (%) | 2 | 1 | 0 | 1 |
| | (12.5) | (25.0) | (0.0) | (16.7) |
| Pts with Serious TEAEs and | 1 | 1 | 0 | 0 |
| Related to a compound of | (6.3) | (25.0) | (0.0) | (0.0) |
| Formula I, n (%) | | | | |
| Pts with Serious TEAEs and | 1 | 1 | 0 | 0 |
| Related to Pembrolizumab, n | (6.3) | (25.0) | (0.0) | (0.0) |
| % | | | | |
| Pts with Fatal TEAEs, n (%) | 0 | 0 | 0 | 0 |
| | (0.0) | (0.0) | (0.0) | (0.0) |
| Pts with Fatal TEAEs and | 0 | 0 | 0 | 0 |
| Related to a compound of | (0.0) | (0.0) | (0.0) | (0.0) |
| Formula I, n (%) | | | | |
| Pts with Fatal TEAEs and | 0 | 0 | 0 | 0 |
| Related to Pembrolizumab, n | (0.0) | (0.0) | (0.0) | (0.0) |
| (%) | | | | |

As can be seen in Table 11 above, Phase 1b Cohort B4 patients did not experience a fatal TEAE related to a compound of Formula I or a fatal TEAE related to pembrolizumab.

TABLE 11A
Preliminary NCT04956640 Phase 1b Cohort B4 data of Treatment-
Emergent Adverse Events by Starting Dose of a compound of Formula I

| | Total | 50 mg BID | 100 mg BID | 150 mg BID |
|---|---|---|---|---|
| | (N = 25) | (N = 5) | (N = 14) | (N = 6) |
| Patients (Pts) with TEAEs, n (%) | 20 | 5 | 9 | 6 |
| | (80.0) | (100.0) | (64.3) | (100.0) |
| Pts with TEAEs Related to a | 16 | 4 | 6 | 6 |
| compound of Formula I, n (%) | (64.0) | (80.0) | (42.9) | (100.0) |
| Pts with TEAEs Related to | 18 | 5 | 7 | 6 |
| Pembrolizumab, n (%) | (72.0) | (100) | (50.0) | (100.0) |
| Pts with TEAEs Maximum | 9 | 2 | 3 | 4 |
| Severity 3 or 4, n (%) | (36.0) | (40.0) | (21.4) | (66.7) |
| Pts with TEAEs Maximum | 6 | 2 | 2 | 2 |
| Severity 3 or 4 and Related to a | (24.0) | (40.0) | (14.3) | (33.3) |
| compound of Formula I, n (%) | | | | |
| Pts with TEAEs Maximum | 6 | 2 | 2 | 2 |
| Severity 3 or 4 and Related to | (24.0) | (40.0) | (14.3) | (33.3) |
| Pembrolizumab, n (%) | | | | |
| Pts with a compound of Formula | 7 | 2 | 3 | 2 |
| I, and/or Pembrolizumab related | (28.0) | (40.0) | (21.4) | (33.3) |
| grade >=3 TEAEs, n (%) | | | | |
| Pts with Serious TEAEs, n (%) | 4 | 1 | 1 | 2 |
| | (16.0) | (20.0) | (7.1) | (33.3) |
| Pts with Serious TEAEs and | 1 | 1 | 0 | 0 |
| Related to a compound of | (4.0) | (20.0) | (0.0) | (0.0) |
| Formula I, n (%) | | | | |
| Pts with Serious TEAEs and | 2 | 1 | 1 | 0 |
| Related to Pembrolizumab, n | (8.0) | (20.0) | (0.0) | (0.0) |
| (%) | | | | |
| Pts with Fatal TEAEs, n (%) | 0 | 0 | 0 | 0 |
| | (0.0) | (0.0) | (0.0) | (0.0) |
| Pts with Fatal TEAEs and | 0 | 0 | 0 | 0 |
| Related to a compound of | (0.0) | (0.0) | (0.0) | (0.0) |
| Formula I, n (%) | | | | |
| Pts with Fatal TEAEs and | 0 | 0 | 0 | 0 |
| Related to Pembrolizumab, n | (0.0) | (0.0) | (0.0) | (0.0) |
| (%) | | | | |

TABLE 12
Preliminary NCT04956640 Phase 1b Cohort C2 data of Treatment-
Emergent Adverse Events by Starting Dose of a compound of Formula I

| | Total | 100 mg BID | 150 mg BID |
|---|---|---|---|
| | (N = 20) | (N = 13) | (N = 7) |
| Patients (Pts) with TEAEs, | 18 | 11 | 7 |
| n (%) | (90.0) | (84.6) | (100.0) |
| Pts with TEAEs Related to a | 10 | 6 | 4 |
| compound of Formula I, n | (50.0) | (46.2) | (57.1) |
| (%) | | | |
| Pts with TEAEs Related to | 15 | 9 | 6 |
| Cetuximab, n (%) | (75.0) | (69.2) | (85.7) |
| Pts with TEAEs Maximum | 3 | 2 | 1 |
| Severity 3 or 4, n (%) | (15.0) | (15.4) | (14.3) |
| Pts with TEAEs Maximum | 2 | 2 | 0 |
| Severity 3 or 4 and Related to | (10.0) | (15.4) | (0.0) |
| a compound of Formula I, n | | | |
| (%) | | | |
| Pts with TEAEs Maximum | 1 | 1 | 0 |
| Severity 3 or 4 and Related to | (5.0) | (7.7) | (0.0) |
| Cetuximab, n (%) | | | |
| Pts with a compound of | 2 | 2 | 0 |
| Formula I and/or Cetuximab | (10.0) | (15.4) | (0.0) |
| related grade >=3 TEAEs, n | | | |
| (%) | | | |
| Pts with Serious TEAEs, n | 1 | 0 | 1 |
| (%) | (5.0) | (0.0) | (14.3) |
| Pts with Serious TEAEs and | 0 | 0 | 0 |
| Related to a compound of | (0.0) | (0.0) | (0.0) |
| Formula I, n (%) | | | |
| Pts with Serious TEAEs and | 0 | 0 | 0 |
| Related to Cetuximab, n (%) | (0.0) | (0.0) | (0.0) |
| Pts with Fatal TEAEs, n (%) | 0 | 0 | 0 |
| | (0.0) | (0.0) | (0.0) |
| Pts with Fatal TEAEs and | 0 | 0 | 0 |
| Related to a compound of | (0.0) | (0.0) | (0.0) |
| Formula I, n (%) | | | |
| Pts with Fatal TEAEs and | 0 | 0 | 0 |
| Related to Cetuximab, n (%) | (0.0) | (0.0) | (0.0) |

As can be seen in Table 12 above, Phase 1b Cohort C2 patients did not experience a serious TEAE related to a compound of Formula I. As can be seen in Table 12 above, Phase 1b Cohort C2 patients did not experience a serious TEAE related to cetuximab. As can be seen in Table 12 above, Phase 1b Cohort C2 patients did not experience a fatal TEAE.

As can be seen in Tables 10-12 above that as of January 2023, four Phase 1a, Phase 1b Cohort B4, and Phase 1b Cohort C2 patients had a grade 3 or greater TEAE. No DLTs were observed and MTD was not reached.

As of January 2023, one patient among Phase 1a, Phase 1b Cohort B4, and Phase 1b Cohort C2 experienced a dose reduction to TRAE. As of January 2023, one patient among Phase 1a, Phase 1b Cohort B4, and Phase 1b Cohort C2 experienced permanent discontinuation due to TRAE.

Tables 13, 14, and 15 illustrate preliminary NCT04956640 Phase 1a, Phase 1b Cohort B4, and Phase 1b Cohort C2 data as of January 2023, respectively, of TRAEs by Starting Dose of a compound of Formula I and by Severity.

TABLE 13
Preliminary NCT04956640 Phase 1a data of TRAEs by Starting Dose of
a compound of Formula I and by Severity

| | Patients (Pts) with a compound of Formula I related TEAEs by | | | | |
|---|---|---|---|---|---|
| | severity, n (%) | | | | |
| | | | 100 mg | 150 mg | 200 mg |
| | Total | 50 mg BID | BID | BID | BID |
| | (N = 84) | (N = 15) | (N = 29) | (N = 27) | (N = 13) |
| Total | 58 | 6 | 19 | 21 | 12 |
| | (69.0) | (40.0) | (65.5) | (77.8) | (92.3) |
| Grade 1 | 56 | 6 | 19 | 21 | 10 |
| | (66.7) | (40.0) | (65.5) | (77.8) | (76.9) |
| Grade 2 | 14 | 0 | 7 | 5 | 2 |
| | (16.7) | (0.0) | (24.1) | (18.5) | (15.4) |
| Grade 3 | 4 | 0 | 0 | 2 | 2 |
| | (4.8) | (0.0) | (0.0) | (7.4) | (15.4) |
| Grade 4 | 0 | 0 | 0 | 0 | 0 |
| | (0.0) | (0.0) | (0.0) | (0.0) | (0.0) |
| Grade 5 | 0 | 0 | 0 | 0 | 0 |
| | (0.0) | (0.0) | (0.0) | (0.0) | (0.0) |

As can be seen in Table 13 above, Phase 1a patients did not experience a Grade 4 or Grade 5 TEAE related to a compound of Formula I.

TABLE 14
Preliminary NCT04956640 Phase 1b Cohort B4 data of TRAEs by
Starting Dose of a compound of Formula I and by Severity

| | Patients (Pts) with TEAEs by severity, n (%) | | | |
|---|---|---|---|---|
| | Total | 50 mg BID | 100 mg BID | 150 mg BID |
| | (N = 16) | (N = 4) | (N = 6) | (N = 6) |
| Pts with a compound of | 11 | 2 | 3 | 6 |
| Formula I, Related | (68.8) | (50.0) | (50.0) | (100.0) |
| TEAEs, Total, n (%) | | | | |
| Grade 1, n (%) | 10 | 2 | 2 | 6 |
| | (62.5) | (50.0) | (33.3) | (100.0) |
| Grade 2, n (%) | 4 | 0 | 1 | 3 |
| | (25.0) | (0.0) | (16.7) | (50.0) |
| Grade 3, n (%) | 4 | 1 | 1 | 2 |
| | (25.0) | (25.0) | (16.7) | (33.3) |
| Grade 4, n (%) | 1 | 0 | 0 | 1 |
| | (6.3) | (0.0) | (0.0) | (16.7) |
| Grade 5, n (%) | 0 | 0 | 0 | 0 |
| | (0.0) | (0.0) | (0.0) | (0.0) |
| Pts with Pembrolizumab | 11 | 2 | 3 | 6 |
| related TEAEs, Total, | (68.8) | (50.0) | (50.0) | (100.0) |
| n (%) | | | | |
| Grade 1, n (%) | 11 | 2 | 3 | 6 |
| | (68.8) | (50.0) | (50.0) | (100.0) |
| Grade 2, n (%) | 3 | 0 | 0 | 3 |
| | (18.8) | (0.0) | (0.0) | (50.0) |
| Grade 3, n (%) | 3 | 1 | 0 | 2 |
| | (18.8) | (25.0) | (0.0) | (33.3) |
| Grade 4, n (%) | 1 | 0 | 0 | 1 |
| | (6.3) | (0.0) | (0.0) | (16.7) |
| Grade 5, n (%) | 0 | 0 | 0 | 0 |
| | (0.0) | (0.0) | (0.0) | (0.0) |
| Pts with a compound of | 12 | 2 | 4 | 6 |
| Formula I, and/or | (75.0) | (50.0) | (66.7) | (100.0) |
| Pembrolizumab related | | | | |
| TEAEs, Total, n (%) | | | | |
| Grade 1, n (%) | 11 | 2 | 3 | 6 |
| | (68.8) | (50.0) | (50.0) | (100.0) |
| Grade 2, n (%) | 5 | 0 | 1 | 4 |
| | (31.3) | (0.0) | (16.7) | (66.7) |
| Grade 3, n (%) | 4 | 1 | 1 | 2 |
| | (25.0) | (25.0) | (16.7) | (33.3) |
| Grade 4, n (%) | 1 | 0 | 0 | 1 |
| | (6.3) | (0.0) | (0.0) | (16.7) |
| Grade 5, n (%) | 0 | 0 | 0 | 0 |
| | (0.0) | (0.0) | (0.0) | (0.0) |

As can be seen in Table 14 above, Phase 1b Cohort B4 patients did not experience a Grade 5 TEAE. No DLTs were observed among Phase 1b Cohort B4 patients. Delayed AST/ALT ratio elevations were observed at 150 mg BID among Phase 1b Cohort B4 patients. There were no dose reductions at 50 mg and 100 mg BID doses among Phase 1b Cohort B4 patients. There was one discontinuation due to TRAE at 50 mg BID.

TABLE 15
Preliminary NCT04956640 Phase 1b Cohort C2 data of TRAEs by
Starting Dose of a compound of Formula I and by Severity

| | Patients (Pts) with TEAEs by | | |
|---|---|---|---|
| | severity, n (%) | | |
| | Total | 100 mg BID | 150 mg BID |
| | (N = 20) | (N = 13) | (N = 7) |
| Pts with a compound of | 10 | 6 | 4 |
| Formula I related TEAEs, Total, | (50.0) | (46.2) | (57.1) |
| n (%) | | | |
| Grade 1, n (%) | 10 | 6 | 4 |
| | (50.0) | (46.2) | (57.1) |
| Grade 2, n (%) | 7 | 4 | 3 |
| | (35.0) | (30.8) | (42.9) |
| Grade 3, n (%) | 2 | 2 | 0 |
| | (10.0) | (15.4) | (0.0) |
| Grade 4, n (%) | 0 | 0 | 0 |
| | (0.0) | (0.0) | (0.0) |
| Grade 5, n (%) | 0 | 0 | 0 |
| | (0.0) | (0.0) | (0.0) |
| Pts with Cetuximab related | 15 | 9 | 6 |
| TEAEs, n (%) | (75.0) | (69.2) | (85.7) |
| Grade 1, n (%) | 14 | 8 | 6 |
| | (70.0) | (61.5) | (85.7) |
| Grade 2, n (%) | 10 | 6 | 4 |
| | (50.0) | (46.2) | (57.1) |
| Grade 3, n (%) | 1 | 1 | 0 |
| | (5.0) | (7.7) | (0.0) |
| Grade 4, n (%) | 0 | 0 | 0 |
| | (0.0) | (0.0) | (0.0) |
| Grade 5, n (%) | 0 | 0 | 0 |
| | (0.0) | (0.0) | (0.0) |
| Pts with a compound of | 15 | 9 | 6 |
| Formula I and/or Cetuximab | (75.0) | (69.2) | (85.7) |
| related TEAEs, Total, n (%) | | | |
| Grade 1, n (%) | 14 | 8 | 6 |
| | (70.0) | (61.5) | (85.7) |
| Grade 2, n (%) | 12 | 7 | 5 |
| | (60.0) | (53.8) | (71.4) |
| Grade 3, n (%) | 2 | 2 | 0 |
| | (10.0) | (15.4) | (0.0) |
| Grade 4, n (%) | 0 | 0 | 0 |
| | (0.0) | (0.0) | (0.0) |
| Grade 5, n (%) | 0 | 0 | 0 |
| | (0.0) | (0.0) | (0.0) |

As can be seen in Table 15 above, Phase 1b Cohort C2 patients did not experience a Grade 4 TEAE or a Grade 5 TEAE. One DLT, a grade 3 ALT/AST ratio increase, was observed at 100 mg BID among Phase 1b Cohort C2 patients. 10% of Phase 1b Cohort C2 patients dose reduced a compound of Formula I due to TRAEs. No Phase 1b Cohort C2 patient permanently discontinued due to TRAEs.

Seven Phase 1a, Phase 1b Cohort B4, or Phase 1b Cohort C2 patients previously discontinued a prior KRAS G12C inhibitor due to toxicity. These seven patients have experienced less than or equal to grade 2 TRAEs due to the compound of Formula I. Each of these seven patients are continuing on treatment.

Tables 16, 17, and 18 illustrate preliminary NCT04956640 Phase 1a, Phase 1b Cohort B4, and Phase 1b Cohort C2 data as of January 2023, respectively, of Best Overall Response, Objective Response Rate, and Disease Control Rate by Starting Dose of a compound of Formula I. Table 16A illustrates preliminary NCT04956640 Phase 1a data of Best Overall Response, Objective Response Rate, and Disease Control Rate by Tumor Type of a compound of Formula I. Table 16B illustrates preliminary NCT04956640 Phase 1a data of Best Overall Response, Objective Response Rate, and Disease Control Rate of a compound of Formula I, for NSCLC patients with or without prior KRAS G12C inhibitor usage.

TABLE 16
Preliminary NCT04956640 Phase 1a data of Best Overall Response,
Objective Response Rate, and Disease Control Rate by Starting Dose of a compound of
Formula I

| | Patients (Pts) with a compound of Formula I, by Starting Dose, n | | | | |
|---|---|---|---|---|---|
| | (%) | | | | |
| | | 50 mg | 100 mg | 150 mg | 200 mg |
| | Total | BID | BID | BID | BID |
| | (N = 75) | (N = 14) | (N = 24) | (N = 25) | (N = 12) |
| Best overall | | | | | |
| response (BOR), n | | | | | |
| (%) | | | | | |
| Complete | 0 | 0 | 0 | 0 | 0 |
| Response (CR) | (0.0) | (0.0) | (0.0) | (0.0) | (0.0) |
| Partial | 19 | 2 | 7 | 6 | 4 |
| Response (PR) | (25.3) | (14.3) | (29.2) | (24.0) | (33.3) |
| Partial | 3 | 0 | 2 | 1 | 0 |
| Response, pending | (4.0) | (0.0) | (8.3) | (4.0) | (0.0) |
| confirmation and | | | | | |
| ongoing (uPR) | | | | | |
| Stable | 43 | 9 | 12 | 16 | 6 |
| Disease (SD) | (57.3) | (64.3) | (50.0) | (64.0) | (50.0) |
| Progressive | 9 | 3 | 2 | 2 | 2 |
| Disease (PD) | (12.0) | (21.4) | (8.3) | (8.0) | (16.7) |
| No | 1 | 0 | 1 | 0 | 0 |
| Evaluable (NE) | (1.3) | (0.0) | (4.2) | (0.0) | (0.0) |
| Objective response | | | | | |
| rate (CR + PR) | | | | | |
| Number of | 19 | 2 | 7 | 6 | 4 |
| patients, n (%) | (25.3) | (14.3) | (29.2) | (24.0) | (33.3) |
| 95% | (16.0, 36.7) | (1.8, 42.8) | (12.6, 51.1) | (9.4, 45.1) | (9.9, 65.1) |
| confidence interval | | | | | |
| Objective response | | | | | |
| rate, confirmed | | | | | |
| and unconfirmed | | | | | |
| (CR + PR + uPR) | | | | | |
| Number of | 22 | 2 | 9 | 7 | 4 |
| patients, n (%) | (29.3) | (14.3) | (37.5) | (28.0) | (33.3) |
| 95% | (19.4, 41.0) | (1.8, 42.8) | (18.8, 59.4) | (12.1, 49.4) | (9.9, 65.1) |
| confidence interval | | | | | |
| Disease control | | | | | |
| rate (CR + PR + | | | | | |
| uPR + SD) | | | | | |
| Number of | 65 | 11 | 21 | 23 | 10 |
| patients, n (%) | (86.7) | (78.6) | (87.5) | (92.0) | (83.3) |
| 95% | (76.8, 93.4) | (49.2, 95.3) | (67.6, 97.3) | (74.0, 99.0) | (51.6, 97.9) |
| confidence interval | | | | | |

As can be seen in Table 16 above, responses have been observed at each starting dose level of a compound of Formula I: 50 mg BID, 100 mg BID, 150 mg BID, and 200 mg BID.

TABLE 16A
Preliminary NCT04956640 Phase 1a data of Best Overall Response,
Objective Response Rate, and Disease Control Rate by Tumor Type of a compound of
Formula I

| | Patients (Pts) with a compound of Formula I, by Tumor Type, n (%) | | | | |
|---|---|---|---|---|---|
| | | | | Pancreatic | |
| | Total | NSCLC | CRC | Cancer | Other |
| | (N = 75) | (N = 22) | (N = 20) | (N = 12) | (N = 21) |
| Best overall | | | | | |
| response (BOR), n | | | | | |
| (%) | | | | | |
| Complete | 0 | 0 | 0 | 0 | 0 |
| Response (CR) | (0.0) | (0.0) | (0.0) | (0.0) | (0.0) |
| Partial | 19 | 4 | 1 | 4 | 10 |
| Response (PR) | (25.3) | (18.2) | (5.0) | (33.3) | (47.6) |
| Partial | 3 | 0 | 1 | 1 | 1 |
| Response, pending | (4.0) | (0.0) | (5.0) | (8.3) | (4.8) |
| confirmation and | | | | | |
| ongoing (uPR) | | | | | |
| Stable | 43 | 12 | 16 | 6 | 9 |
| Disease (SD) | (57.3) | (54.5) | (80.0) | (50.0) | (42.9) |
| Progressive | 9 | 5 | 2 | 1 | 1 |
| Disease (PD) | (12.0) | (22.7) | (10.0) | (8.3) | (4.8) |
| No | 1 | 1 | 0 | 0 | 0 |
| Evaluable (NE) | (1.3) | (4.5) | (0.0) | (0.0) | (0.0) |
| Objective response | | | | | |
| rate (CR + PR) | | | | | |
| Number of | 19 | 4 | 1 | 4 | 10 |
| patients, n (%) | (25.3) | (18.2) | (5.0) | (33.3) | (47.6) |
| 95% | (16.0, 36.7) | (5.2, 40.3) | (0.1, 24.9) | (9.9, 65.1) | (25.7, 70.2) |
| confidence interval | | | | | |
| Objective response | | | | | |
| rate, confirmed | | | | | |
| and unconfirmed | | | | | |
| (CR + PR + uPR) | | | | | |
| Number of | 22 | 4 | 2 | 5 | 11 |
| patients, n (%) | (29.3) | (18.2) | (10.0) | (41.7) | (52.4) |
| 95% | (19.4, 41.0) | (5.2, 40.3) | (1.2, 31.7) | (15.2, 72.3) | (29.8, 74.3) |
| confidence interval | | | | | |
| Disease control | | | | | |
| rate (CR + PR + | | | | | |
| uPR + SD) | | | | | |
| Number of | 65 | 16 | 18 | 11 | 20 |
| patients, n (%) | (86.7) | (72.7) | (90.0) | (91.7) | (95.2) |
| 95% | (76.8, 93.4) | (49.8, 89.3) | (68.3, 98.8) | (61.5, 99.8) | (76.2, 99.9) |
| confidence interval | | | | | |

Other Tumor Types include Biliary Tract, Cholangiocarcinoma, Chondrosarcoma, Distal CBD cancer, Duodenal Carcinoma, Jejunal Adenocarcinoma, Large Cell Neuroendocrine of Lung, Nasal Malignant Melanoma, Ovarian, Salivary Adenoid Cystic Carcinoma, Small Intestine, Tracheal Basaloid Squamous Cell Carcinoma, and Upper Tract Urothelial Carcinoma.

TABLE 16B
NSCLC Patients (Pts) with a compound of Formula I, with or without
prior KRAS G12C inhibitor usage

| | NSCLC Patients (Pts) with a compound of Formula I, with or | | |
|---|---|---|---|
| | without prior KRAS G12C inhibitor usage, n (%) | | |
| | | With Prior | Without Prior KRAS |
| Best overall response | Total | KRAS G12Ci | G12Ci |
| (BOR), n (%) | (N = 22) | (N = 14) | (N = 8) |
| Complete | 0 | 0 | 0 |
| Response (CR) | (0.0) | (0.0) | (0.0) |
| Partial Response | 4 | 1 | 3 |
| (PR) | (18.2) | (7.1) | (37.5) |
| Partial Response, | 0 | 0 | 0 |
| pending confirmation | (0.0) | (0.0) | (0.0) |
| and ongoing (uPR) | | | |
| Stable Disease | 12 | 8 | 4 |
| (SD) | (54.5) | (57.1) | (50.0) |
| Progressive | 5 | 4 | 1 |
| Disease (PD) | (22.7) | (28.6) | (12.5) |
| No Evaluable | 1 | 1 | 0 |
| (NE) | (4.5) | (7.1) | (0.0) |
| Objective response rate | | | |
| (CR + PR) | | | |
| Number of | 4 | 1 | 3 |
| patients, n (%) | (18.2) | (7.1) | (37.5) |
| 95% confidence | (5.2, 40.3) | (0.2, 33.9) | (8.5, 75.5) |
| interval | | | |
| Objective response rate, | | | |
| confirmed and | | | |
| unconfirmed (CR + PR + | | | |
| uPR) | | | |
| Number of | 4 | 1 | 3 |
| patients, n (%) | (18.2) | (7.1) | (37.5) |
| 95% confidence | (5.2, 40.3) | (0.2, 33.9) | (8.5, 75.5) |
| interval | | | |
| Disease control rate | | | |
| (CR + PR + uPR + SD) | | | |
| Number of | 16 | 9 | 7 |
| patients, n (%) | (72.7) | (64.3) | (87.5) |
| 95% confidence | (49.8, 89.3) | (35.1, 87.2) | (47.3, 99.7) |
| interval | | | |

TABLE 17
Preliminary NCT04956640 Phase 1b Cohort B4 data of Best Overall
Response, Objective Response Rate, and Disease Control Rate by Starting Dose of a
compound of Formula I

| | Patients (Pts) with a compound of Formula I, by | | | |
|---|---|---|---|---|
| | Starting Dose, n (%) | | | |
| Characteristic | Total | 50 mg BID | 100 mg BID | 150 mg BID |
| Statistic/Category | (N = 13) | (N = 1) | (N = 6) | (N = 6) |
| Best overall response (BOR), n | | | | |
| (%) | | | | |
| Complete Response | 0 | 0 | 0 | 0 |
| (CR) | (0.0) | (0.0) | (0.0) | (0.0) |
| Partial Response (PR) | 5 | 0 | 2 | 3 |
| | (38.5) | (0.0) | (33.3) | (50.0) |
| Partial Response, | 4 | 0 | 2 | 2 |
| pending confirmation and | (30.8) | (0.0) | (33.3) | (33.3) |
| ongoing (uPR) | | | | |
| Stable Disease (SD) | 3 | 0 | 2 | 1 |
| | (23.1) | (0.0) | (33.3) | (16.7) |
| Progressive Disease | 1 | 1 | 0 | 0 |
| (PD) | (7.7) | (100.0) | (0.0) | (0.0) |
| Not Evaluable (NE) | 0 | 0 | 0 | 0(0.0) |
| Objective response rate (CR + | (0.0) | (0.0) | (0.0) | |
| PR) | | | | |
| Number of patients, n | 5 | 0 | 2 | 3 |
| (%) | (38.5) | (0.0) | (33.3) | (50.0) |
| 95% confidence | (13.9, 68.4) | (0.0, 97.5) | (4.3, 77.7) | (11.8, 88.2) |
| interval | | | | |
| Objective response rate, | | | | |
| confirmed and unconfirmed | | | | |
| (CR + PR + uPR) | | | | |
| Number of patients, n | 9 | 0 | 4 | 5 |
| %) | (69.2) | (0.0) | (66.7) | (83.3) |
| 95% confidence | (38.6, 90.9) | (0.0, 97.5) | (22.3, 95.7) | (35.9, 99.6) |
| interval | | | | |
| Disease control rate (CR + | | | | |
| PR + IPR + SD) | | | | |
| Number of patients, n | 12 | 0 | 6 | 6 |
| (%) | (92.3) | (0.0) | (100.0) | (100.0) |
| 95% confidence | (64.0, 99.8) | (0.0, 97.5) | (54.1, 100.0) | (54.1, 100.0) |
| interval | | | | |

As can be seen in Table 17 above, responses have been observed at starting dose levels of a compound of Formula I: 100 mg BID, and 150 mg BID.

TABLE 18
preliminary NCT04956640 Phase 1b Cohort C2 data of Best Overall
Response, Objective Response Rate, and Disease Control Rate by Starting Dose of a
compound of Formula I

| | Patients (Pts) with a compound of Formula I, by | | |
|---|---|---|---|
| | Starting Dose, n (%) | | |
| | Total | 100 mg BID | 150 mg BID |
| | (N = 11) | (N = 6) | (N = 5) |
| Best overall response (BOR), n (%) | | | |
| Complete Response (CR) | 0 (0.0) | 0 (0.0) | 0 (0.0) |
| Partial Response (PR) | 3 (27.3) | 3 (50.0) | 0 (0.0) |
| Partial Response, pending | 2 (18.2) | 0 (0.0) | 2 (40.0) |
| confirmation and ongoing (uPR) | | | |
| Stable Disease (SD) | 6 (54.5) | 3 (50.0) | 3 (60.0) |
| Progressive Disease (PD) | 0 (0.0) | 0 (0.0) | 0 (0.0) |
| Not Evaluable (NE) | 0 (0.0) | 0 (0.0) | 0 (0.0) |
| Objective response rate (CR + PR) | | | |
| Number of patients, n (%) | 3 (27.3) | 3 (50.0) | 0 (0.0) |
| 95% confidence interval | (6.0, 61.0) | (11.8, 88.2) | (0.0, 52.2) |
| Objective response rate, confirmed | | | |
| and unconfirmed (CR + PR + uPR) | | | |
| Number of patients, n (%) | 5 (45.5) | 3 (50.0) | 2 (40.0) |
| 95% confidence interval | (16.7, 76.6) | (11.8, 88.2) | (5.3, 85.3) |
| Disease control rate (CR + PR + | | | |
| iPR + SD) | | | |
| Number of patients, n (%) | 11 (100.0) | 6 (100.0) | 5 (100.0) |
| 95% confidence interval | (71.5, 100.0) | (54.1, 100.0) | (47.8, 100.0) |

As can be seen in Table 18 above, responses have been observed at both starting dose levels of a compound of Formula c:100 mg BID, and 150 mg BID.

Tables 19, 20, and 21 illustrate preliminary NCT04956640 Phase 1a, Phase 1b Cohort B4, and Phase 1b Cohort C2 data as of January 2023, respectively, of Duration of Response Summary by Starting Dose of a compound of Formula I.

TABLE 19
Preliminary NCT04956640 Phase 1a data of Duration of Response
Summary by Starting Dose of a compound of Formula I

| | Patients (Pts) with a compound of Formula I, by Starting Dose, n | | | | |
|---|---|---|---|---|---|
| | (%) | | | | |
| | Total | 50 mg BID | 100 mg BID | 150 mg BID | 200 mg BID |
| | (N = 75) | (N = 14) | (N = 24) | (N = 25) | (N = 12) |
| Patients with best | 19 (25.3) | 2 (14.3) | 7 (29.2) | 6 (24.0) | 4 (33.3) |
| overall response of | | | | | |
| CR or PR, N′ (%) | | | | | |
| Duration of | | | | | |
| response (DOR), n (%) | | | | | |
| <6 months | 16 (84.2) | 2 (100.0) | 7 (100.0) | 4 (66.7) | 3 (75.0) |
| 6 to <9 months | 3 (15.8) | 0 (0.0) | 0 (0.0) | 2 (33.3) | 1 (25.0) |
| Duration of | | | | | |
| response (months)¹ | | | | | |
| Median | 6.24 | NE | 5.45 | 7.21 | 6.93 |
| 95% confidence interval | 5.45, 8.18 | NE, NE | 2.86, 5.45 | 6.24, 8.18 | NE, NE |
| Duration of follow-up | | | | | |
| (months)¹ | | | | | |
| Median | 4.30 | 4.96 | 4.17 | 4.17 | 4.37 |
| 95% confidence interval | 2.79, 5.62 | 4.30, 5.62 | 1.41, NE | 1.35, NE | 2.79, NE |

¹Estimates based on Kaplan-Meier method with 95% confidence intervals calculated using Brookmeyer and Crowley method.

Median time on treatment among Phase 1a patients was 3.8 months.

TABLE 20
Preliminary NCT04956640 Phase 1a, Phase 1b Cohort B4, and Phase 1b
Cohort C2 data as of January 2023, respectively, of TRAEs by Starting Dose of a
compound of Formula I and by Severity

| | Patients (Pts) with a compound of Formula I, by | | | |
|---|---|---|---|---|
| | Starting Dose, n (%) | | | |
| | Total | 50 mg BID | 100 mg BID | 150 mg BID |
| | (N = 13) | (N = 1) | (N = 6) | (N = 6) |
| Patients with best overall | 5 (38.5) | 0 (0.0) | 2 (33.3) | 3 (50.0) |
| response of CR or PR, N′ (%) | | | | |
| Duration of response (DOR), n (%) | | | | |
| <6 months | 5 (100.0) | 0 (NE) | 2 (100.0) | 3 (100.0) |

Median time on treatment among Phase 1b Cohort B4 patients was 2.5 months.

TABLE 21
Preliminary NCT04956640 Phase 1b Cohort C2 data of Duration of
Response Summary by Starting Dose of a compound of Formula I

| | Patients (Pts) with a compound of | | |
|---|---|---|---|
| | Formula I, by Starting Dose, n (%) | | |
| | Total | 100 mg BID | 150 mg BID |
| | (N = 11) | (N = 6) | (N = 5) |
| Patients with best overall response | 3 (27.3) | 3 (50.0) | 0 (0.0) |
| of CR or PR, N′ (%) | | | |
| Overall response status, n (%) | | | |
| Censored | 3 (100.0) | 3 (100.0) | 0 (NE) |
| Reason censored, n (%) | | | |
| Alive without documented | 3 (100.0) | 3 (100.0) | 0 (NE) |
| disease progression | | | |
| Duration of response (DOR), n (%) | | | |
| <6 months | 3 (100.0) | 3 (100.0) | 0 (NE) |

Tables 22, 23, and 24 illustrate preliminary NCT04956640 Phase 1a, Phase 1b Cohort B4, and Phase 1b Cohort C2 data as of January 2023, respectively, of Time to Response by Starting Dose of a compound of Formula I.

TABLE 22
Preliminary NCT04956640 Phase 1a data of Time to Response by
Starting Dose of a compound of Formula I

| | Patients (Pts) with a compound of Formula I, by Starting | | | | |
|---|---|---|---|---|---|
| | Dose, n (%) | | | | |
| | Total | 50 mg BID | 100 mg BID | 150 mg BID | 200 mg BID |
| | (N = 75) | (N = 14) | (N = 24) | (N = 25) | (N = 12) |
| Patients with best overall | 19 (25.3) | 2 (14.3) | 7 (29.2) | 6 (24.0) | 4 (33.3) |
| response of CR or PR, N′ (%) | | | | | |
| Time to response (TTR), n (%) | | | | | |
| <2 months | 14 (73.7) | 2 (100.0) | 5 (71.4) | 4 (66.7) | 3 (75.0) |
| 2 to <4 months | 2 (10.5) | 0 (0.0) | 1 (14.3) | 0 (0.0) | 1 (25.0) |
| 4 to <6 months | 2 (10.5) | 0 (0.0) | 0 (0.0) | 2 (33.3) | 0 (0.0) |
| 6 to <9 months | 1 (5.3) | 0 (0.0) | 1 (14.3) | 0 (0.0) | 0 (0.0) |
| Time to response (months) | | | | | |
| Median | 1.41 | 1.25 | 1.41 | 1.46 | 1.33 |
| Interquartile range | 1.45 | 0.13 | 1.68 | 2.69 | 0.87 |
| Min, Max | 1.2, 7.7 | 1.2, 1.3 | 1.3, 7.7 | 1.4, 4.2 | 1.2, 2.8 |

As can be seen in Table 22 above, Phase 1a patients experienced a 1.4 month median time to response.

TABLE 23
Preliminary NCT04956640 Phase 1b Cohort B4 data of Time to
Response by Starting Dose of a compound of Formula I

| | Patients (Pts) with a compound of | | | |
|---|---|---|---|---|
| | Formula I, by Starting Dose, n (%) | | | |
| | | 50 mg | 100 mg | 150 mg |
| | Total | BID | BID | BID |
| | (N = 13) | (N = 1) | (N = 6) | (N = 6) |
| Patients with best overall | 5 (38.5) | 0 (0.0) | 2 (33.3) | 3 (50.0) |
| response of CR or PR, N′ (%) | | | | |
| Time to response (TTR), n (%) | | | | |
| <2 months | 4 (80.0) | 0 (NE) | 2 (100.0) | 2 (66.7) |
| 2 to <4 months | 1 (20.0) | 0 (NE) | 0 (0.0) | 1 (33.3) |
| Time to response (months) | | | | |
| Median | 1.38 | NE | 1.22 | 1.41 |
| Interquartile range | 0.07 | NE | 0.26 | 1.61 |
| Min, Max | 1.1, 3.0 | NE, NE | 1.1, 1.3 | 1.4, 3.0 |

As can be seen in Table 23 above, Phase 1b Cohort B4 patients experienced a 1.4 month median time to response.

TABLE 24
Preliminary NCT04956640 Phase 1b Cohort C2 data of Time to
Response by Starting Dose of a compound of Formula I

| | Patients (Pts) with a compound of | | |
|---|---|---|---|
| | Formula I, by Starting Dose, n (%) | | |
| | | 100 mg | 150 mg |
| | Total | BID | BID |
| | (N = 11) | (N = 6) | (N = 5) |
| Patients with best overall response | 3 (27.3) | 3 (50.0) | 0 (0.0) |
| of CR or PR, N′ (%) | | | |
| Time to response (TTR), n (%) | | | |
| <2 months | 3 (100.0) | 3 (100.0) | 0 (NE) |
| Time to response (months) | | | |
| Median | 1.31 | 1.31 | NE |
| Interquartile range | 0.33 | 0.33 | NE |
| Min, Max | 1.2, 1.6 | 1.2, 1.6 | NE, NE |

As can be seen in Table 24 above, Phase 1b Cohort C2 patients experienced a 1.3 month median time to response.

Phase 1a, Phase 1b Cohort B4, and Phase 1b Cohort C2 patients experienced a 1.4 month median time to response. At a median follow-up of 4.2 months, 55% of Phase 1a, Phase 1b Cohort B4, and Phase 1b Cohort C2 patients remain ongoing. 100% of Phase 1b Cohort B4 patients remain ongoing.

Tables 25, 26, and 27 illustrate preliminary NCT04956640 Phase 1a, Phase 1b Cohort B4, and Phase 1b Cohort C2 data as of January 2023, respectively, by Starting Dose of the compound of Formula I, by prior KRAS inhibitor (“KRAS G12Ci”), by % Change from Baseline, and by Best Response.

TABLE 25
Preliminary NCT04956640 Phase 1a data by Starting Dose of the
compound of Formula I, by prior KRAS inhibitor (“KRAS G12Ci”),
by % Change from Baseline, and by Best Response

| | Starting | Prior | % Change from Baseline in | |
|---|---|---|---|---|
| | Dose | KRAS | Sum of Diameters for all | Best |
| Patient | (BID) | G12Ci² | Target Lesions | Response |
| 1 | 50 | Y | 50.0 | PD |
| 2 | 150 | | 27.3 | PD |
| 3 | 50 | | 9.7 | PD |
| 4 | 200 | Y | 5.3 | PD |
| 5 | 150 | | 4.3 | SD |
| 6 | 150 | | 3.8 | SD |
| 7 | 150 | | 3.4 | SD |
| 8 | 200 | Y | 1.4 | SD |
| 9 | 100 | | 0.7 | SD |
| 10 | 100 | | 0.5 | SD |
| 11 | 150 | | 0.0 | SD |
| 12 | 50 | | −0.3 | SD |
| 13 | 150 | | −0.7 | SD |
| 14 | 150 | Y | −0.7 | SD |
| 15 | 150 | Y | −3.3 | PD |
| 16 | 200 | | −4.2 | SD |
| 17 | 100 | Y | −4.3 | SD |
| 18 | 100 | | −4.8 | SD |
| 19 | 200 | | −5.0 | SD |
| 20 | 150 | | −5.3 | SD |
| 21 | 100 | | −6.0 | SD |
| 22 | 50 | | −6.0 | SD |
| 23 | 150 | | −7.1 | SD |
| 24 | 100 | | −8.1 | SD |
| 25 | 150 | | −8.2 | SD |
| 26 | 50 | | −8.6 | SD |
| 27 | 200 | | −9.6 | PD |
| 28 | 200 | | −9.9 | SD |
| 29 | 50 | | −9.9 | SD |
| 30 | 50 | | −10.5 | SD |
| 31 | 150 | | −11.5 | SD |
| 32 | 50 | Y | −13.0 | SD |
| 33 | 50 | | −13.1 | SD |
| 34 | 200 | | −14.3 | SD |
| 35 | 150 | | −16.7 | SD |
| 36 | 50 | | −17.1 | SD |
| 37 | 100 | | −17.3 | PD |
| 38 | 150 | | −18.8 | SD |
| 39 | 100 | | −21.1 | SD |
| 40 | 100 | | −22.4 | PD |
| 41 | 100 | | −22.7 | SD |
| 42 | 150 | | −23.3 | SD |
| 43 | 150 | | −24.0 | SD |
| 44 | 100 | | −25.1 | SD |
| 45 | 150 | Y | −25.8 | SD |
| 46 | 50 | | −26.8 | SD |
| 47 | 150 | Y | −29.7 | SD |
| 48 | 100 | | −29.8 | PR Pending |
| | | | | Confirmation |
| | | | | and Ongoing |
| 49 | 100 | | −30.0 | SD |
| 50 | 50 | | −31.6 | PR Confirmed |
| 51 | 100 | | −32.3 | SD |
| 52 | 200 | | −32.3 | PR Confirmed |
| 53 | 100 | | −32.3 | PR Pending |
| | | | | Confirmation |
| | | | | and Ongoing |
| 54 | 100 | | −35.4 | PR Confirmed |
| 55 | 150 | | −37.0 | PR Pending |
| | | | | Confirmation |
| | | | | and Ongoing |
| 56 | 200 | | −37.5 | PR Confirmed |
| 57 | 100 | Y | −40.0 | SD |
| 58 | 150 | | −41.6 | PR Confirmed |
| 59 | 100 | Y | −44.7 | PR Confirmed |
| 60 | 50 | | −45.0 | PR Confirmed |
| 61 | 150 | | −51.0 | PR Confirmed |
| 62 | 100 | | −51.4 | PR Confirmed |
| 63 | 150 | | −52.4 | PR Confirmed |
| 64 | 100 | | −54.0 | PR Confirmed |
| 65 | 100 | | −54.8 | PR Confirmed |
| 66 | 100 | | −57.1 | PR Confirmed |
| 67 | 150 | | −60.3 | PR Confirmed |
| 68 | 200 | | −65.9 | PR Confirmed |
| 69 | 150 | | −67.9 | PR Confirmed |
| 70 | 200 | | −75.0 | PR Confirmed |
| 71 | 100 | | −84.4 | PR Confirmed |
| 72 | 150 | | −91.1 | PR Confirmed |

²If patient has had at least one prior KRAS G12Ci then Y. If patient has not had a prior KRAS G12Ci then blank.

A patient discontinued treatment before the first post-baseline assessment and is therefore not presented in Table 25. Two patients have post-baseline assessments, but not G all target lesions were assessed as of data extraction and are therefore not presented in Table 25.

As can be seen in Table 25 above, Phase 1a patients with prior KRAS G12Ci experienced PD, SD, and confirmed PR. Median time on treatment among Phase 1a patients is 3.8 months.

TABLE 26
Preliminary NCT04956640 Phase 1b Cohort B4 data by Starting Dose
of the compound of Formula I, by prior KRAS inhibitor (“KRAS G12Ci”),
by % Change from Baseline, and by Best Response

| | Starting | Prior | % Change from Baseline in | |
|---|---|---|---|---|
| | Dose | KRAS | Sum of Diameters for all | |
| Patient | (BID) | G12Ci² | Target Lesions | Best Response |
| 1 | 150 | Y | 7.3 | SD |
| 2 | 50 | Y | 1.1 | PD |
| 3 | 100 | | 0.0 | SD |
| 4 | 100 | Y | 0.0 | SD |
| 5 | 150 | | −29.5 | PR Pending |
| | | | | Confirmation |
| | | | | and Ongoing |
| 6 | 150 | Y | −32.1 | PR Confirmed |
| 7 | 150 | | −38.2 | PR Confirmed |
| 8 | 100 | | −40.9 | PR Pending |
| | | | | Confirmation |
| | | | | and Ongoing |
| 9 | 100 | | −45.1 | PR Pending |
| | | | | Confirmation |
| | | | | and Ongoing |
| 10 | 150 | | −49.2 | PR Confirmed |
| 11 | 150 | | −57.7 | SD |
| 12 | 100 | | −59.4 | PR Confirmed |
| 13 | 100 | | −82.8 | PR Confirmed |

Median time on treatment among Phase 1b Cohort B4 patients is 2.5 months.

TABLE 27
Preliminary NCT04956640 Phase 1b Cohort C2 data by Starting Dose
of the compound of Formula I, by prior KRAS inhibitor (“KRAS G12Ci”),
by % Change from Baseline, and by Best Response

| | Starting | Prior | % Change from Baseline in | |
|---|---|---|---|---|
| | Dose | KRAS | Sum of Diameters for all | |
| Patient | (BID) | G12Ci² | Target Lesions | Best Response |
| 1 | 150 | | −5.7 | SD |
| 2 | 150 | | −13.6 | SD |
| 3 | 150 | | −19.4 | SD |
| 4 | 100 | | −22.0 | SD |
| 5 | 100 | | −23.4 | SD |
| 6 | 150 | | −36.7 | PR Pending |
| | | | | Confirmation |
| | | | | and Ongoing |
| 7 | 100 | | −38.9 | PR Confirmed |
| 8 | 150 | | −45.7 | PR Pending |
| | | | | Confirmation |
| | | | | and Ongoing |
| 9 | 100 | | −48.7 | PR Confirmed |
| 10 | 100 | | −55.4 | PR Confirmed |
| 11 | 100 | | −67.6 | SD |

Median time on treatment among Phase 1b Cohort C2 patients is 1.8 months.

Tables 28, 29, and 30 illustrate preliminary NCT04956640 Phase 1a, Phase 1b Cohort B4, and Phase 1b Cohort C2 data as of January 2023, respectively, by Starting Dose of the compound of Formula I, by prior KRAS inhibitor (“KRAS G12Ci”), by Time, and by Parameter wherein the Parameter identifies a condition experienced at the corresponding Time.

TABLE 28
Preliminary NCT04956640 Phase 1a data by Starting Dose of the
compound of Formula I, by prior KRAS inhibitor (“KRAS G12Ci”), by Time, and by
Parameter wherein the Parameter identifies a condition experienced at the corresponding Time

| | Starting | Prior | | |
|---|---|---|---|---|
| | Dose | KRAS | Time | |
| Patient | (BID) | G12Ci² | (months) | Parameter |
| 1 | 100 | | 1.4 | Partial Response - Confirmed |
| 1 | 100 | | 4.9 | Time on Treatment |
| 2 | 50 | | 6.7 | Progressive Disease |
| 2 | 50 | | 6.7 | Time on Treatment |
| 2 | 50 | | 9.8 | Death |
| 3 | 100 | | 1.2 | Progressive Disease |
| 3 | 100 | | 1.4 | Time on Treatment |
| 3 | 100 | | 1.7 | Death |
| 4 | 100 | | 1.3 | Progressive Disease |
| 4 | 100 | | 2.8 | Time on Treatment |
| 5 | 150 | Y | 9.2 | Time on Treatment |
| 6 | 200 | | 2.5 | Progressive Disease |
| 6 | 200 | | 2.7 | Time on Treatment |
| 7 | 200 | | 1.3 | Progressive Disease |
| 7 | 200 | | 1.4 | Time on Treatment |
| 7 | 200 | | 4.7 | Death |
| 8 | 100 | Y | 0.3 | Time on Treatment |
| 9 | 50 | Y | 1.3 | Progressive Disease |
| 9 | 50 | Y | 3.6 | Time on Treatment |
| 9 | 50 | Y | 8.4 | Death |
| 10 | 200 | | 1.4 | Partial Response - Confirmed |
| 10 | 200 | | 7.6 | Time on Treatment |
| 10 | 200 | | 8.3 | Progressive Disease |
| 11 | 100 | | 1.4 | Partial Response - Confirmed |
| 11 | 100 | | 6.7 | Time on Treatment |
| 12 | 200 | Y | 4.4 | Time on Treatment |
| 13 | 100 | Y | 6.4 | Time on Treatment |
| 14 | 100 | Y | 3.1 | Partial Response - Confirmed |
| 14 | 100 | Y | 5.4 | Time on Treatment |
| 15 | 200 | | 7.4 | Progressive Disease |
| 15 | 200 | | 7.6 | Time on Treatment |
| 16 | 50 | | 2.8 | Progressive Disease |
| 16 | 50 | | 2.8 | Time on Treatment |
| 16 | 50 | | 5.7 | Death |
| 17 | 50 | | 1.2 | Progressive Disease |
| 17 | 50 | | 1.3 | Time on Treatment |
| 17 | 50 | | 1.8 | Death |
| 18 | 150 | | 6.3 | Time on Treatment |
| 19 | 200 | | 1.2 | Partial Response - Confirmed |
| 19 | 200 | | 5.8 | Time on Treatment |
| 20 | 50 | | 5.3 | Progressive Disease |
| 20 | 50 | | 7.2 | Time on Treatment |
| 20 | 50 | | 7.4 | Death |
| 21 | 150 | | 7.8 | Time on Treatment |
| 22 | 50 | | 2.7 | Progressive Disease |
| 22 | 50 | | 4.3 | Time on Treatment |
| 22 | 50 | | 11.5 | Death |
| 23 | 150 | | 11.7 | Time on Treatment |
| 24 | 50 | | 11.1 | Progressive Disease |
| 24 | 50 | | 11.1 | Time on Treatment |
| 25 | 150 | Y | 4.1 | Progressive Disease |
| 25 | 150 | Y | 4.2 | Time on Treatment |
| 26 | 200 | Y | 3.0 | Time on Treatment |
| 27 | 100 | | 2.0 | Time on Treatment |
| 28 | 50 | Y | 1.0 | Time on Treatment |
| 28 | 50 | Y | 1.0 | Progressive Disease |
| 28 | 50 | Y | 1.8 | Death |
| 29 | 150 | Y | 1.2 | Progressive Disease |
| 29 | 150 | Y | 1.3 | Time on Treatment |
| 29 | 150 | Y | 3.3 | Death |
| 30 | 50 | | 17.2 | Time on Treatment |
| 31 | 100 | | 9.5 | Progressive Disease |
| 31 | 100 | | 9.5 | Time on Treatment |
| 32 | 150 | | 1.4 | Partial Response - Confirmed |
| 32 | 150 | | 4.6 | Time on Treatment |
| 33 | 100 | | 7.7 | Partial Response - Confirmed |
| 33 | 100 | | 13.4 | Time on Treatment |
| 34 | 150 | | 2.3 | Time on Treatment |
| 35 | 50 | | 1.3 | Partial Response - Confirmed |
| 35 | 50 | | 7.1 | Time on Treatment |
| 36 | 200 | Y | 1.3 | Progressive Disease |
| 36 | 200 | Y | 2.8 | Time on Treatment |
| 37 | 50 | | 1.2 | Partial Response - Confirmed |
| 37 | 50 | | 7.1 | Time on Treatment |
| 38 | 150 | | 2.1 | Time on Treatment |
| 38 | 150 | | 3.3 | Progressive Disease |
| 38 | 150 | | 4.9 | Death |
| 39 | 150 | | 1.4 | Time on Treatment |
| 40 | 200 | | 1.2 | Partial Response - Confirmed |
| 40 | 200 | | 6.7 | Time on Treatment |
| 41 | 100 | | 4.6 | Time on Treatment |
| 42 | 150 | Y | 2.3 | Time on Treatment |
| 43 | 100 | | 2.3 | Time on Treatment |
| 44 | 150 | | 1.2 | Progressive Disease |
| 44 | 150 | | 1.3 | Time on Treatment |
| 45 | 100 | | 2.3 | Time on Treatment |
| 46 | 150 | | 1.4 | Time on Treatment |
| 47 | 50 | | 2.8 | Time on Treatment |
| 47 | 50 | | 2.8 | Progressive Disease |
| 48 | 50 | | 7.2 | Time on Treatment |
| 49 | 100 | | 2.8 | Time on Treatment |
| 49 | 100 | | 2.9 | Progressive Disease |
| 50 | 150 | | 1.5 | Partial Response - Confirmed |
| 50 | 150 | | 9.7 | Progressive Disease |
| 50 | 150 | | 10.1 | Time on Treatment |
| 51 | 150 | | 4.1 | Partial Response - Confirmed |
| 51 | 150 | | 11.2 | Time on Treatment |
| 52 | 150 | | 6.7 | Progressive Disease |
| 52 | 150 | | 6.9 | Time on Treatment |
| 53 | 100 | | 1.1 | Time on Treatment |
| 54 | 150 | | 4.2 | Partial Response - Confirmed |
| 54 | 150 | | 5.7 | Time on Treatment |
| 55 | 100 | | 5.5 | Partial Response - Pending |
| | | | | Confirmation and Ongoing |
| 55 | 100 | | 5.7 | Time on Treatment |
| 56 | 150 | | 1.4 | Partial Response - Confirmed |
| 56 | 150 | | 3.9 | Time on Treatment |
| 57 | 100 | | 2.6 | Time on Treatment |
| 57 | 100 | | 2.7 | Progressive Disease |
| 58 | 150 | | 2.8 | Time on Treatment |
| 59 | 150 | | 2.3 | Time on Treatment |
| 60 | 100 | | 1.3 | Partial Response - Confirmed |
| 60 | 100 | | 4.1 | Time on Treatment |
| 60 | 100 | | 4.2 | Progressive Disease |
| 60 | 100 | | 7.0 | Death |
| 61 | 50 | Y | 5.5 | Time on Treatment |
| 61 | 50 | Y | 5.6 | Progressive Disease |
| 62 | 150 | | 11.7 | Time on Treatment |
| 63 | 150 | | 1.4 | Partial Response - Confirmed |
| 63 | 150 | | 7.6 | Progressive Disease |
| 63 | 150 | | 9.6 | Time on Treatment |
| 64 | 100 | | 1.4 | Partial Response - Confirmed |
| 64 | 100 | | 6.9 | Progressive Disease |
| 64 | 100 | | 7.1 | Time on Treatment |
| 64 | 100 | | 10.3 | Death |
| 65 | 100 | Y | 2.8 | Time on Treatment |
| 65 | 100 | Y | 2.8 | Progressive Disease |
| 65 | 100 | Y | 4.2 | Death |
| 66 | 100 | | 4.1 | Time on Treatment |
| 66 | 100 | | 4.1 | Progressive Disease |
| 66 | 100 | | 7.6 | Death |
| 67 | 200 | | 7.1 | Time on Treatment |
| 68 | 200 | | 2.8 | Time on Treatment |
| 68 | 200 | | 2.9 | Progressive Disease |
| 69 | 150 | | 4.1 | Time on Treatment |
| 69 | 150 | | 4.1 | Progressive Disease |
| 69 | 150 | | 5.3 | Death |
| 70 | 200 | | 2.8 | Partial Response - Confirmed |
| 70 | 200 | | 7.3 | Time on Treatment |
| 71 | 100 | | 1.4 | Partial Response - Confirmed |
| 71 | 100 | | 5.5 | Time on Treatment |
| 71 | 100 | | 5.6 | Progressive Disease |
| 72 | 150 | | 2.9 | Time on Treatment |
| 73 | 100 | | 2.3 | Time on Treatment |
| 74 | 100 | | 1.2 | Partial Response - Pending |
| | | | | Confirmation and Ongoing |
| 74 | 100 | | 2.6 | Time on Treatment |
| 75 | 150 | | 1.2 | Partial Response - Pending |
| | | | | Confirmation and Ongoing |
| 75 | 150 | | 2.6 | Time on Treatment |

TABLE 29
Preliminary NCT04956640 Phase 1b Cohort B4 data by Starting Dose
of the compound of Formula I, by prior KRAS inhibitor (″KRAS G12Ci″),
by Time, and by Parameter wherein the Parameter identifies a condition
experienced at the corresponding Time

| | Starting | Prior | | |
|---|---|---|---|---|
| | Dose | KRAS | Time | |
| Patient | (BID) | G12Ci² | (months) | Parameter |
| 1 | 100 | | 5.6 | Time on Treatment |
| 2 | 50 | Y | 1.1 | Progressive Disease |
| 2 | 50 | Y | 1.1 | Time on Treatment |
| 3 | 100 | | 1.3 | Partial Response - Confirmed |
| 3 | 100 | | 6.0 | Time on Treatment |
| 4 | 150 | | 1.4 | Partial Response - Confirmed |
| 4 | 150 | | 4.4 | Time on Treatment |
| 5 | 100 | | 1.3 | Partial Response - Pending |
| | | | | Confirmation and Ongoing |
| 5 | 100 | | 1.8 | Time on Treatment |
| 6 | 100 | Y | 1.8 | Time on Treatment |
| 7 | 150 | Y | 3.7 | Time on Treatment |
| 8 | 150 | Y | 1.1 | Partial Response - Pending |
| | | | | Confirmation and Ongoing |
| 8 | 150 | Y | 2.9 | Time on Treatment |
| 9 | 100 | | 1.4 | Partial Response - Pending |
| | | | | Confirmation and Ongoing |
| 9 | 100 | | 2.1 | Time on Treatment |
| 10 | 150 | | 3.0 | Partial Response - Confirmed |
| 10 | 150 | | 4.5 | Time on Treatment |
| 11 | 100 | | 1.1 | Partial Response - Confirmed |
| 11 | 100 | | 5.5 | Time on Treatment |
| 12 | 150 | | 1.4 | Partial Response - Confirmed |
| 12 | 150 | | 4.1 | Time on Treatment |
| 13 | 150 | | 1.0 | Time on Treatment |

TABLE 30
Preliminary NCT04956640 Phase 1b Cohort C2 data by Starting Dose
of the compound of Formula I, by prior KRAS inhibitor (“KRAS G12Ci”),
by Time, and by Parameter wherein the Parameter identifies a condition
experienced at the corresponding Time

| | Starting | Prior | | |
|---|---|---|---|---|
| | Dose | KRAS | Time | |
| Patient | (BID) | G12Ci² | (months) | Parameter |
| 1 | 100 | | 3.4 | Time on Treatment |
| 2 | 100 | | 1.3 | Partial Response - Confirmed |
| 2 | 100 | | 4.1 | Time on Treatment |
| 3 | 100 | | 3.7 | Time on Treatment |
| 4 | 150 | | 2.3 | Time on Treatment |
| 5 | 100 | | 1.6 | Partial Response - Confirmed |
| 5 | 100 | | 3.4 | Time on Treatment |
| 6 | 100 | | 1.2 | Partial Response - Confirmed |
| 6 | 100 | | 4.6 | Time on Treatment |
| 7 | 150 | | 2.0 | Time on Treatment |
| 8 | 150 | | 2.1 | Partial Response - Pending |
| | | | | Confirmation and Ongoing |
| 8 | 150 | | 2.3 | Time on Treatment |
| 9 | 150 | | 1.6 | Time on Treatment |
| 10 | 100 | | 4.4 | Progressive Disease |
| 10 | 100 | | 5.1 | Time on Treatment |
| 11 | 150 | | 1.4 | Partial Response - Pending |
| | | | | Confirmation and Ongoing |
| 11 | 150 | | 2.5 | Time on Treatment |

Tables 31, 32, and 33 illustrate preliminary NCT04956640 Phase 1a, Phase 1b Cohort B4, and Phase 1b Cohort C2 data as of April 2023, respectively, of Treatment Duration by Starting Dose of the compound of Formula I.

TABLE 31
Preliminary NCT04956640 Phase 1a data as of April 2023 of Treatment
Duration by Starting Dose of the compound of Formula I

| | Total | 50 mg BID | 100 mg BID | 150 mg BID | 200 mg BID |
|---|---|---|---|---|---|
| | (N = 109) | (N = 16) | (N = 32) | (N = 48) | (N = 13) |
| Time on Treatment (TOT) in | 5.16 | 6.65 | 5.11 | 4.50 | 5.91 |
| Months of the compound of | (4.1) | (5.5) | (3.6) | (4.0) | (3.2) |
| Formula I, Mean (SD) | | | | | |
| TOT in Months of the | 4.11 | 4.99 | 4.16 | 2.91 | 7.10 |
| compound of Formula I, | | | | | |
| Median | | | | | |
| TOT in Months of the | 2.37, 7.16 | 2.78, 9.05 | 2.78, 6.75 | 1.66, 6.21 | 2.83, 8.18 |
| compound of Formula I, Q1, | | | | | |
| Q3 | | | | | |
| TOT in Months of the | 0.20, 21.03 | 0.99, 21.03 | 0.26, 17.22 | 0.20, 15.54 | 1.41, 10.48 |
| compound of Formula I, Min, | | | | | |
| Max | | | | | |
| Time on Study (TOS) in | 6.88 | 9.74 | 6.71 | 5.31 | 9.57 |
| Months, Mean (SD) | (4.6) | (5.8) | (3.5) | (4.4) | (3.7) |
| TOS in Months, Median | 6.08 | 8.95 | 6.67 | 3.58 | 10.48 |
| TOS in Months, Q1, Q3 | 2.99, 10.12 | 5.11, 13.21 | 3.84, 9.35 | 1.97, 6.80 | 8.18, 12.75 |
| TOS in Months, Min, Max | 0.89, 21.03 | 1.77, 21.03 | 1.28, 17.22 | 0.89, 15.54 | 3.02, 13.77 |

TABLE 32
Preliminary NCT04956640 Phase 1b Cohort B4 data as of April 2023 of
Treatment Duration by Starting Dose of the compound of Formula I

| | Total | 50 mg BID | 100 mg BID | 150 mg BID |
|---|---|---|---|---|
| | (N = 25) | (N = 5) | (N = 14) | (N = 6) |
| Time on Treatment (TOT) in Months | 4.17 | 3.54 | 3.81 | 5.53 |
| of the the compound of Formula I, Mean (SD) | (3.0) | (1.4) | (3.5) | (2.6) |
| TOT in Months of the compound of | 4.04 | 4.04 | 2.53 | 5.95 |
| Formula I, Median | | | | |
| TOT in Months of the compound of | 1.41, | 2.79, | 0.85, | 4.86, |
| Formula I, Q1, Q3 | 5.91 | 4.67 | 5.91 | 7.13 |
| TOT in Months of the compound of | 0.33, | 1.41, | 0.33, | 0.95, |
| Formula I, Min, Max | 9.79 | 4.76 | 9.79 | 8.34 |
| Time on Treatment (TOT) in Months of | 3.76 | 2.86 | 3.74 | 4.54 |
| Pembrolizumab, Mean (SD) | (3.0) | (1.6) | (3.5) | (2.9) |
| TOT in Months of Pembrolizumab, Median | 2.66 | 2.79 | 2.30 | 5.19 |
| TOT in Months of Pembrolizumab, | 1.25, | 2.10, | 0.85, | 1.45, |
| Q1, Q3 | 5.62 | 4.04 | 5.91 | 6.37 |
| TOT in Months of Pembrolizumab, | 0.33, | 0.72, | 0.33, | 0.72, |
| Min, Max | 9.79 | 4.67 | 9.79 | 8.34 |
| Time on Treatment (TOT) in Months | 3.76 | 2.86 | 3.74 | 4.54 |
| of Combination, Mean (SD) | (3.0) | (1.6) | (3.5) | (2.9) |
| TOT in Months of Combination, Median | 2.66 | 2.79 | 2.30 | 5.19 |
| TOT in Months of Combination, Q1, Q3 | 1.25, | 2.10, | 0.85, | 1.45, |
| | 5.62 | 4.04 | 5.91 | 6.37 |
| TOT in Months of Combination, Min, Max | 0.33, | 0.72, | 0.33, | 0.72, |
| | 9.79 | 4.67 | 9.79 | 8.34 |
| Time on Treatment (TOT) in Months | 4.17 | 3.54 | 3.81 | 5.53 |
| of any treatment, Mean (SD) | (3.0) | (1.4) | (3.5) | (2.6) |
| TOT in Months of any treatment, Median | 4.04 | 4.04 | 2.53 | 5.95 |
| TOT in Months of any treatment, Q1, Q3 | 1.41, | 2.79, | 0.85, | 4.86, |
| | 5.91 | 4.67 | 5.91 | 7.13 |
| TOT in Months of any treatment, Min, Max | 0.33, | 1.41, | 0.33, | 0.95, |
| | 9.79 | 4.76 | 9.79 | 8.34 |
| Time on Study (TOS) in Months, Mean (SD) | 4.76 | 4.42 | 4.02 | 6.75 |
| | (3.0) | (0.8) | (3.5) | (1.7) |
| TOS in Months, Median | 4.76 | 4.67 | 2.53 | 7.34 |
| TOS in Months, Q1, Q3 | 2.40, | 4.04, | 0.85, | 4.86, |
| | 6.74 | 4.76 | 5.91 | 8.18 |
| TOS in Months, Min, Max | 0.33, | 3.19, | 0.33, | 4.44, |
| | 9.79 | 5.42 | 9.79 | 8.34 |

In summary, Phase 1a patients well tolerated the compound of Formula I. The compound of Formula I demonstrated preliminary efficacy across dose levels and in multiple tumor types among Phase 1a patients.

Phase 1b Cohort B4 patients and Phase 1b Cohort C2 patients demonstrate surprisingly promising safety profiles. Phase 1b Cohort B4 patient safety data at 50 mg BID and 100 mg BID is notable for low rate of immune-related AEs, including hepatoxicity, with 3 Phase 1b Cohort B4 patients treated for greater than or equal to 4 months.

Embodiment 1. A method of treating a KRAS G12C mutant cancer comprising: administering to a patient in need of such treatment, a dose between about 50 mg and about 200 mg of a compound of Formula I:

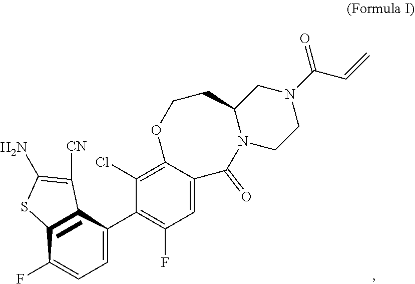

or a pharmaceutically acceptable salt thereof.

Embodiment 2. The method of embodiment 1, wherein the dose between about 50 mg and about 200 mg is of the compound of Formula I.

Embodiment 3. The method of embodiment 1, wherein the dose is a maximum daily dose selected from the group consisting of 50 mg, 100 mg, 150 mg, 200 mg, 300 mg, 400 mg, 500 mg, 600 mg, 700 mg, and 800 mg.

Embodiment 4. The method of embodiment 2, wherein the maximum daily dose is about 50 mg.

Embodiment 5. The method of embodiment 2, wherein the maximum daily dose is about 100 mg.

Embodiment 6. The method of embodiment 2, wherein the maximum daily dose is about 200 mg.

Embodiment 7. The method of embodiment 2, wherein the maximum daily dose is about 300 mg.

Embodiment 8. The method of embodiment 2, wherein the maximum daily dose is about 400 mg.

Embodiment 8-1. A method of treating a KRAS G12C mutant cancer comprising administering to a patient in need of such treatment, a dose between about 50 mg and about 200 mg of a compound of Formula I.

Embodiment 9. The method of any one of embodiments 1-8, wherein the KRAS G12C mutant cancer is selected from the group consisting of lung cancer, colorectal cancer, pancreatic cancer, bladder cancer, cervical cancer, endometrial cancer, ovarian cancer, cholangiocarcinoma, and esophageal cancer.

Embodiment 10. The method of any one of embodiments 1-9, wherein the KRAS G12C mutant cancer is non-small cell lung cancer, pancreatic cancer, or colorectal cancer.

Embodiment 11. The method of any one of embodiments 1-10, wherein the KRAS G12C mutant cancer is non-small cell lung cancer or colorectal cancer.

Embodiment 12. The method of any one of embodiments 1-9, wherein the KRAS G12C mutant cancer is non-small cell lung cancer.

Embodiment 13. The method of any one of embodiments 1-9, wherein the KRAS G12C mutant cancer is pancreatic cancer.

Embodiment 14. The method of any one of embodiments 1-9, wherein the KRAS G12C mutant cancer is colorectal cancer.

Embodiment 15. The method of any one of embodiments 1 to 11, wherein the dose is administered as a dose between about 50 mg and about 200 mg to the patient at least once a day.

Embodiment 16. The method of embodiment 15, wherein the dose is selected from the group consisting of about 50 mg, about 100 mg, about 150 mg, and about 200 mg.

Embodiment 17. The method of any one of embodiments 1 to 16, wherein the dose is in a capsule containing about 5 mg, about 25 mg, or about 50 mg of the compound of Formula I, or a pharmaceutically acceptable salt thereof.

Embodiment 18. The method of any one of embodiments 1 to 16, wherein the dose is administered as a dose between about 50 mg and about 200 mg to the patient at least twice a day.

Embodiment 19. The method of any one of embodiments 1 to 18, wherein the dose is administered as a dose of about 50 mg to the patient at least twice a day.

Embodiment 20. The method of any one of embodiments 1 to 18, wherein the dose is administered as a dose of about 100 mg to the patient at least twice a day.

Embodiment 21. The method of any one of embodiments 1 to 18, wherein the dose is administered as a dose of about 150 mg to the patient at least twice a day.

Embodiment 22. The method of any one of embodiments 1 to 18, wherein the dose is administered as a dose of about 200 mg to the patient at least twice a day.

Embodiment 23. The method of any one of embodiments 1 to 22, wherein the step of administering the dose occurs up to the end of the patient's life.

Embodiment 24. The method of any one of embodiments 1 to 23, comprising:
monitoring the patient for a DLT; and
administering a second dose of the compound of Formula I, or a pharmaceutically acceptable salt thereof, if the patient exhibits the DLT, wherein the second dose is reduced as compared to a first dose.

Embodiment 25. A method of treating a KRAS G12C mutant cancer comprising:
(a) administering to a patient in need of such treatment a first dose between about 50 mg to about 200 mg of the compound of Formula I, or a pharmaceutically acceptable salt thereof,
(b) monitoring the patient for a DLT; and
(c) administering a second dose of the compound of Formula I, or a pharmaceutically acceptable salt thereof, if the patient exhibits the DLT, wherein the second dose is reduced as compared to the first dose.

Embodiment 26. The method of embodiment 25, wherein the first dose is selected from the group consisting of about 50 mg, about 100 mg, about 150 mg, and about 200 mg.

Embodiment 27. The method of embodiments 25 or 26, wherein the second dose is selected from the group consisting of 5 mg, 10 mg, 15 mg, 20 mg, 25 mg, 30 mg, 35 mg, 40 mg, 45 mg, 50 mg, 55 mg, 6 mg, 65 mg, 70 mg, 75 mg, 80 mg, 85 mg, 90 mg, 95 mg, 100 mg, 110 mg, 120 mg, 130 mg, 140 mg, and about 150 mg.

Embodiment 28. The method of any one of embodiments 25 to 27, wherein the second dose is selected from the group consisting of about 50 mg, about 100 mg, and about 150 mg.

Embodiment 29. The method of any one of embodiments 24 to 28, wherein the DLT includes a treatment-emergent adverse event and a clinically significant determination.

Embodiment 30. The method of any one of embodiments 24 to 29, wherein the DLT occurs during the first 21 days of administering the first dose.

Embodiment 31. The method of embodiments 29 or 30, wherein the treatment-emergent adverse event is hematologic toxicity, febrile neutropenia, hepatotoxicity, mucositis, diarrhea, ocular toxicity, QT prolongation, confirmed ILD, pneumonitis, constipation, nausea or vomiting, fatigue, AST/ALT ratio, creatinine elevations, elevated or increased bilirubin, neutropenia, anemia, or thrombocytopenia.

Embodiment 32. The method of any one of embodiments 29 to 31, wherein the treatment-emergent adverse event is hematologic toxicity.

Embodiment 33. The method of any one of embodiments 29 to 31, wherein the treatment-emergent adverse event is febrile neutropenia.

Embodiment 34. The method of any one of embodiments 29 to 31, wherein the treatment-emergent adverse event is hepatotoxicity.

Embodiment 35. The method of any one of embodiments 29 to 31, wherein the treatment-emergent adverse event is mucositis.

Embodiment 36. The method of any one of embodiments 29 to 31, wherein the treatment-emergent adverse event is diarrhea.

Embodiment 37. The method of any one of embodiments 29 to 31, wherein the treatment-emergent adverse event is ocular toxicity.

Embodiment 38. The method of any one of embodiments 29 to 31, wherein the treatment-emergent adverse event is QT prolongation.

Embodiment 39. The method of any one of embodiments 29 to 31, wherein the treatment-emergent adverse event is confirmed ILD.

Embodiment 40. The method of any one of embodiments 29 to 31, wherein the treatment-emergent adverse event is pneumonitis.

Embodiment 41. The method of any one of embodiments 29 to 31, wherein the treatment-emergent adverse event is constipation.

Embodiment 42. The method of any one of embodiments 29 to 31, wherein the treatment-emergent adverse event is nausea or vomiting.

Embodiment 43. The method of any one of embodiments 29 to 31, wherein the treatment-emergent adverse event is fatigue.

Embodiment 44. The method of any one of embodiments 29 to 31, wherein the treatment-emergent adverse event is AST/ALT ratio.

Embodiment 45. The method of any one of embodiments 29 to 31, wherein the treatment-emergent adverse event is creatinine elevations.

Embodiment 46. The method of any one of embodiments 29 to 31, wherein the treatment-emergent adverse event is elevated or increased bilirubin.

Embodiment 47. The method of any one of embodiments 29 to 31, wherein the treatment-emergent adverse event is neutropenia.

Embodiment 48. The method of any one of embodiments 29 to 31, wherein the treatment-emergent adverse event is anemia.

Embodiment 49. The method of any one of embodiments 29 to 31, wherein the treatment-emergent adverse event is thrombocytopenia.

Embodiment 50. The method of any one of embodiments 24 to 49, wherein the second dose is reduced by 50 mg as compared to the first dose except for when the first dose is 50 mg, when the first dose is 50 mg, the second dose is reduced by 5 mg, 10 mg, 15 mg, 20 mg, 25 mg, 30 mg, 35 mg, 40 mg, or 45 mg.

Embodiment 51. The method of any one of embodiments 24 to 49, wherein the second dose is reduced by 100 mg as compared to the first dose except for when the first dose is 50 mg or 100 mg, when the first dose is 50 mg, the second dose is reduced by 5 mg, 10 mg, 15 mg, 20 mg, 25 mg, 30 mg, 35 mg, 40 mg, or 45 mg, when the first dose is 100 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg.

Embodiment 52. The method of any one of embodiments 24 to 49, wherein the second dose is reduced by 150 mg as compared to the first dose except for when the first dose is 50 mg, 100 mg or 150 mg, when the first dose is 50 mg, the second dose is reduced by 5 mg, 10 mg, 15 mg, 20 mg, 25 mg, 30 mg, 35 mg, 40 mg, or 45 mg, when the first dose is 100 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, or 95 mg, when the first dose is 150 mg, the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, or 145 mg.

Embodiment 53. The method of any one of embodiments 24 to 49, wherein the second dose is reduced by 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, or 195 mg as compared to the first dose.

Embodiment 54. The method of any one of embodiments 25 to 53, further comprising the steps of:
(a) administering to a patient in need of such treatment the second dose selected from the group consisting of about 50 mg, about 100 mg, and about 150 mg of the compound of Formula I, or a pharmaceutically acceptable salt thereof,
(b) monitoring the patient for a DLT; and
(c) administering a third dose of the compound of Formula I, or a pharmaceutically acceptable salt thereof, if the patient exhibits a DLT, wherein the third dose is reduced as compared to the second dose.

Embodiment 55. The method of embodiment 54, wherein the DLT includes a treatment-emergent adverse event and a clinically significant determination.

Embodiment 56. The method of embodiment 54, wherein the DLT occurs during the first 21 days of administering the second dose.

Embodiment 57. The method of embodiments 55 or 56, wherein the treatment-emergent adverse event is grade 3 or higher.

Embodiment 58. The method of any one of embodiments 55 to 57, wherein the treatment-emergent adverse event is grade 4.

Embodiment 59. The method of embodiment 54, wherein the third dose is reduced by 50 mg as compared to the second dose.

Embodiment 60. The method of any one of embodiments 1 to 58, wherein after administration of the compound of Formula I, or a pharmaceutically acceptable salt thereof, the patient achieves a partial response.

Embodiment 61. The method of any one of embodiments 1 to 58, wherein after administration of the compound of Formula I, or a pharmaceutically acceptable salt thereof, the patient achieves a complete response.

Embodiment 62. The method of any one of embodiments 1 to 58, wherein after administration of the compound of Formula I, or a pharmaceutically acceptable salt thereof, the patient achieves a stable disease.

Embodiment 63. A method of treating a KRAS G12C mutant cancer comprising:
administering to a patient in need thereof, a dose between about 50 mg and about 200 mg of the compound of Formula I, or a pharmaceutically acceptable salt thereof, in simultaneous, separate or sequential combination with a second therapeutic agent.

Embodiment 64. The method of embodiment 63, wherein the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

Embodiment 65. The method of embodiment 64, wherein the PD-1 or PD-L1 inhibitor is pembrolizumab.

Embodiment 66. The method of embodiment 38, wherein the PD-1 or PD-L1 inhibitor is nivolumab.

Embodiment 67. The method of embodiment 38, wherein the PD-1 or PD-L1 inhibitor is cimiplimab.

Embodiment 68. The method of embodiment 38, wherein the PD-1 or PD-L1 inhibitor is sintilimab.

Embodiment 69. The method of embodiment 38, wherein the PD-1 or PD-L1 inhibitor is atezolizumab.

Embodiment 70. The method of embodiment 38, wherein the PD-1 or PD-L1 inhibitor is avelumab.

Embodiment 71. The method of embodiment 38, wherein the PD-1 or PD-L1 inhibitor is durvalumab.

Embodiment 72. The method of embodiment 38, wherein the PD-1 or PD-L1 inhibitor is lodapilimab.

Embodiment 73. The method of embodiment 38, wherein the CDK4/CDK6 inhibitor is abemaciclib.

Embodiment 74. The method of embodiment 38, wherein the CDK4/CDK6 inhibitor is palbociclib.

Embodiment 75. The method of embodiment 38, wherein the CDK4/CDK6 inhibitor is ribociclib.

Embodiment 76. The method of embodiment 38, wherein the EGFR inhibitor is erlotinib.

Embodiment 77. The method of embodiment 38, wherein the EGFR inhibitor is afatinib.

Embodiment 78. The method of embodiment 38, wherein the EGFR inhibitor is gefitinib.

Embodiment 79. The method of embodiment 38, wherein the EGFR inhibitor is cetuximab.

Embodiment 80. The method of embodiment 38, wherein the ERK inhibitor is Temuterkib.

Embodiment 81. The method of embodiment 38, wherein the ERK inhibitor is LTT462.

Embodiment 82. The method of embodiment 38, wherein the ERK inhibitor is KO-947.

Embodiment 83. The method of embodiment 38, wherein the platinum agent is cisplatin.

Embodiment 84. The method of embodiment 38, wherein the platinum agent is carboplatin.

Embodiment 85. The method of embodiment 38, wherein the platinum agent is oxaliplatin.

Embodiment 86. The method of embodiment 38, wherein the antifolate is pemetrexed.

Embodiment 87. The method of embodiment 38, wherein the Aurora A inhibitor is selected from the group consisting of an Aurora A selective inhibitor, or a pharmaceutically acceptable salt thereof, alisertib, a pan Aurora inhibitor, or a pharmaceutically acceptable salt thereof, tozasertib, danusertib, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

Embodiment 88. The method of embodiment 38, wherein the Aurora A inhibitor is (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt.

Embodiment 89. The method of embodiment 38, wherein the SHP2 inhibitor is selected from the group consisting of a Type I SHP2 inhibitor, a Type II SHP2 inhibitor, BBP-398, IACS-15509, or IACS-13909, X37, ERAS-601, SH3809, HBI-2376, ETS-001, and PCC0208023, or a pharmaceutically acceptable salt thereof.

Embodiment 90. The method of embodiment 89, wherein the Type I SHP2 inhibitor is selected from the group consisting of PHPS1 or GS-493, NSC-87877 or NSC-117199, and cefsulodin, or a pharmaceutically acceptable salt thereof.

Embodiment 91. The method of embodiment 89, wherein the Type II SHP2 inhibitor is selected from the group consisting of JAB-3068 or JAB-3312, RMC-4550 or RMC-4630, SHP099, SHP244, SHP389, SHP394, or TNO155, RG-6433 and RLY-1971, or a pharmaceutically acceptable salt thereof.

Embodiment 92. The method of embodiment 91, wherein the Type II SHP2 inhibitor is selected from the group consisting of JAB-3068, RMC-4630, TNO155, and RLY-1971, or a pharmaceutically acceptable salt thereof.

Embodiment 93. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC.

Embodiment 94. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with abemaciclib in the treatment of KRAS G12C-mutant advanced NSCLC.

Embodiment 95. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with erlotinib in the treatment of KRAS G12C-mutant advanced NSCLC.

Embodiment 96. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with Temuterkib in the treatment of KRAS G12C-mutant advanced NSCLC or CRC.

Embodiment 97. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 98. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with TNO155 in the treatment of KRAS G12C-mutant advanced NSCLC or CRC.

Embodiment 99. A method of treating a KRAS G12C mutant cancer, comprising: administering to a patient in need thereof, a dose between about 50 mg and about 200 mg of the compound of Formula I, or a pharmaceutically acceptable salt thereof, in simultaneous, separate or sequential combination with cetuximab in the treatment of KRAS G12C-mutant advanced CRC.

Embodiment 100. Use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose between about 50 mg and about 200 mg.

Embodiment 101. The use of embodiment 100, wherein the dose is selected from the group consisting of about 50 mg, about 100 mg, about 150 mg, and about 200 mg.

Embodiment 102. Use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose selected from the group consisting of about 50 mg, about 100 mg, about 150 mg, and about 200 mg;
the patient is monitored for DLT; and
if the patient exhibits DLT, a second dose of the compound of Formula I, or a pharmaceutically acceptable salt thereof, is administered, wherein the second dose is reduced as compared to the first dose.

Embodiment 103. Use of the compound of Formula I or a pharmaceutically acceptable salt thereof, in the manufacture of a medicament for treatment of a KRAS G12C mutant cancer, wherein a dose between about 50 mg and about 200 mg of a compound of Formula I or a pharmaceutically acceptable salt thereof is administered to a patient in need thereof, wherein the KRAS G12C mutant cancer is KRAS G12C-mutant advanced NSCLC.

Embodiment 104. Use of the compound of Formula I, or a pharmaceutically acceptable salt thereof, administering to a patient in need thereof, a dose between about 50 mg and about 200 mg of the compound of Formula I, or a pharmaceutically acceptable salt thereof, in simultaneous, separate or sequential combination with a second therapeutic agent.

Embodiment 105. The use of embodiment 103, wherein the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

Embodiment 106. The use of embodiment 105, wherein the dose of the compound of Formula I, or a pharmaceutically acceptable salt thereof, is a reduced dose.

Embodiment 107. The use of embodiment 106, wherein the reduced dose is about 50 mg.

Embodiment 108. The use of embodiment 106, wherein the reduced dose is about 100 mg.

Embodiment 109. The use of embodiment 106, wherein the reduced dose is about 150 mg.

Embodiment 110. The compound of Formula I, or a pharmaceutically acceptable salt thereof, for use in the treatment of a KRAS G12C mutant cancer wherein the dose of the compound administered is between about 50 mg and about 200 mg.

Embodiment 111. The compound of Formula I, or a pharmaceutically acceptable salt thereof, for use in the treatment of a KRAS G12C mutant cancer in a patient wherein:
(a) a first dose selected from the group consisting of about 50 mg, about 100 mg, about 150 mg, and about 200 mg of the compound of Formula I is administered to the patient;
(b) the patient is monitored for DLT; and
(c) if the patient exhibits DLT, a second dose of the compound of Formula I is administered which is reduced as compared to the first dose.

Embodiment 112. The compound for use according to embodiment 111, wherein the first dose is selected from the group consisting of about 50 mg, about 100 mg, about 150 mg, and about 200 mg.

Embodiment 113. The compound for use according to embodiments 111 or 112, wherein the second dose is selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, and about 150 mg.

Embodiment 114. The compound for use according to any one of embodiments 111 to 113, wherein the second dose is selected from the group consisting of about 50 mg, about 100 mg, and about 150 mg.

Embodiment 114. A method of treating a KRAS G12C mutant cancer comprising: administering to a patient in need of such treatment, a dose of about 150 mg of a compound of Formula I:

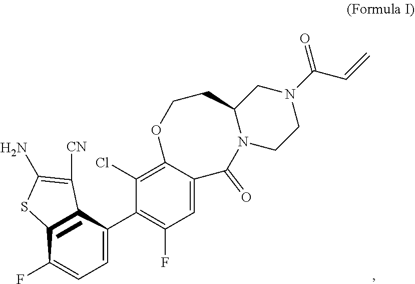

or a pharmaceutically acceptable salt thereof.

Embodiment 115. A method of treating a KRAS G12C mutant cancer comprising administering to a patient in need of such treatment, a dose of about 150 mg of a compound of Formula I.

Embodiment 116. A method of treating a KRAS G12C mutant cancer comprising administering to a patient in need of such treatment, a dose of about 150 mg of a compound of Formula I, wherein the KRAS G12C mutant cancer is KRAS G12C-mutant advanced NSCLC.

Embodiment 117. A method of treating a KRAS G12C mutant cancer, comprising administering to a patient in need of such treatment, a first dose of about 150 mg of the compound of Formula I, or a pharmaceutically acceptable salt thereof, monitoring the patient for a DLT; and administering a second dose of the compound of Formula I, or a pharmaceutically acceptable salt thereof, if the patient exhibits the DLT, wherein the second dose is reduced as compared to the first dose.

Embodiment 118. A method of treating a KRAS G12C mutant cancer comprising administering to a patient in need thereof, a dose of about 150 mg of the compound of Formula I, or a pharmaceutically acceptable salt thereof, in simultaneous, separate or sequential combination with a second therapeutic agent.

Embodiment 119. A method of treating a KRAS G12C mutant cancer comprising administering to a patient in need thereof, a dose of about 100 mg of the compound of Formula I, or a pharmaceutically acceptable salt thereof, in simultaneous, separate or sequential combination with a second therapeutic agent.

Embodiment 120. A method of treating a KRAS G12C mutant cancer comprising administering to a patient in need thereof, a dose of about 50 mg of the compound of Formula I, or a pharmaceutically acceptable salt thereof, in simultaneous, separate or sequential combination with a second therapeutic agent.

Embodiment 121. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC.

Embodiment 122. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC.

Embodiment 123. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC.

Embodiment 124. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with abemaciclib in the treatment of KRAS G12C-mutant advanced NSCLC.

Embodiment 125. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with abemaciclib in the treatment of KRAS G12C-mutant advanced NSCLC.

Embodiment 126. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with abemaciclib in the treatment of KRAS G12C-mutant advanced NSCLC.

Embodiment 127. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with erlotinib in the treatment of KRAS G12C-mutant advanced NSCLC.

Embodiment 128. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with erlotinib in the treatment of KRAS G12C-mutant advanced NSCLC.

Embodiment 129. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with erlotinib in the treatment of KRAS G12C-mutant advanced NSCLC.

Embodiment 130. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with Temuterkib in the treatment of KRAS G12C-mutant advanced NSCLC.

Embodiment 131. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with Temuterkib in the treatment of KRAS G12C-mutant advanced NSCLC.

Embodiment 132. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with Temuterkib in the treatment of KRAS G12C-mutant advanced NSCLC.

Embodiment 133. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with Temuterkib in the treatment of KRAS G12C-mutant advanced CRC.

Embodiment 134. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with Temuterkib in the treatment of KRAS G12C-mutant advanced CRC.

Embodiment 135. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with Temuterkib in the treatment of KRAS G12C-mutant advanced CRC.

Embodiment 136. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 137. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 138. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 139. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced NSCLC.

Embodiment 140. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced NSCLC.

Embodiment 141. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced NSCLC.

Embodiment 142. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with TNO155 in the treatment of KRAS G12C-mutant advanced CRC.

Embodiment 143. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with TNO155 in the treatment of KRAS G12C-mutant advanced CRC.

Embodiment 144. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with TNO155 in the treatment of KRAS G12C-mutant advanced CRC.

Embodiment 145. A method of treating a KRAS G12C mutant cancer, comprising administering to a patient in need thereof, a dose of about 150 mg of the compound of Formula I, or a pharmaceutically acceptable salt thereof, in simultaneous, separate or sequential combination with cetuximab in the treatment of KRAS G12C-mutant advanced CRC.

Embodiment 146. A method of treating a KRAS G12C mutant cancer, comprising administering to a patient in need thereof, a dose of about 100 mg of the compound of Formula I, or a pharmaceutically acceptable salt thereof, in simultaneous, separate or sequential combination with cetuximab in the treatment of KRAS G12C-mutant advanced CRC.

Embodiment 147. A method of treating a KRAS G12C mutant cancer, comprising administering to a patient in need thereof, a dose of about 50 mg of the compound of Formula I, or a pharmaceutically acceptable salt thereof, in simultaneous, separate or sequential combination with cetuximab in the treatment of KRAS G12C-mutant advanced CRC.

Embodiment 148. A use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose of about 150 mg.

Embodiment 149. A use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a dose of about 100 mg.

Embodiment 150. A use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose of about 150 mg; the patient is monitored for DLT; and if the patient exhibits DLT, a second dose of the compound of Formula I, or a pharmaceutically acceptable salt thereof, is administered, wherein the second dose is reduced as compared to the first dose.

Embodiment 151. A use of a compound of Formula I, or a pharmaceutically acceptable salt thereof, in the manufacture of a medicament for treatment of a KRAS G12C mutant cancer, wherein the compound of Formula I, or a pharmaceutically acceptable salt thereof, is administered at a first dose of about 100 mg; the patient is monitored for DLT; and if the patient exhibits DLT, a second dose of the compound of Formula I, or a pharmaceutically acceptable salt thereof, is administered, wherein the second dose is reduced as compared to the first dose.

Embodiment 152. A use of the compound of Formula I or a pharmaceutically acceptable salt thereof, in the manufacture of a medicament for treatment of a KRAS G12C mutant cancer, wherein a dose of about 150 mg of a compound of Formula I or a pharmaceutically acceptable salt thereof is administered to a patient in need thereof, wherein the KRAS G12C mutant cancer is KRAS G12C-mutant advanced NSCLC.

Embodiment 153. A use of the compound of Formula I, or a pharmaceutically acceptable salt thereof, administering to a patient in need thereof, a dose of about 150 mg of the compound of Formula I, or a pharmaceutically acceptable salt thereof, in simultaneous, separate or sequential combination with a second therapeutic agent.

Embodiment 154. A use of the compound of Formula I, or a pharmaceutically acceptable salt thereof, administering to a patient in need thereof, a dose of about 100 mg of the compound of Formula I, or a pharmaceutically acceptable salt thereof, in simultaneous, separate or sequential combination with a second therapeutic agent.

Embodiment 155. A use of the compound of Formula I, or a pharmaceutically acceptable salt thereof, administering to a patient in need thereof, a dose of about 50 mg of the compound of Formula I, or a pharmaceutically acceptable salt thereof, in simultaneous, separate or sequential combination with a second therapeutic agent.

Embodiment 156. A use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with pembrolizumab in the treatment of KRAS G12C-mutant advanced NSCLC.

Embodiment 157. A use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with pembrolizumab in the treatment of KRAS G12C-mutant advanced NSCLC.

Embodiment 158. A use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with pembrolizumab in the treatment of KRAS G12C-mutant advanced NSCLC.

Embodiment 159. A use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with abemaciclib in the treatment of KRAS G12C-mutant advanced NSCLC.

Embodiment 160. A use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with abemaciclib in the treatment of KRAS G12C-mutant advanced NSCLC.

Embodiment 161. A use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with abemaciclib in the treatment of KRAS G12C-mutant advanced NSCLC.

Embodiment 162. A use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with erlotinib in the treatment of KRAS G12C-mutant advanced NSCLC.

Embodiment 163. A use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with erlotinib in the treatment of KRAS G12C-mutant advanced NSCLC.

Embodiment 164. A use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with erlotinib in the treatment of KRAS G12C-mutant advanced NSCLC.

Embodiment 165. A use of the compound of Formula I, or a pharmaceutically acceptable salt thereof, in the manufacture of a medicament for treatment of a KRAS G12C mutant cancer, administering to a patient in need thereof, a dose between about 150 mg of the compound of Formula I, or a pharmaceutically acceptable salt thereof, in simultaneous, separate or sequential combination with Temuterkib in the treatment of KRAS G12C-mutant advanced NSCLC.

Embodiment 166. A use of the compound of Formula I, or a pharmaceutically acceptable salt thereof, in the manufacture of a medicament for treatment of a KRAS G12C mutant cancer, administering to a patient in need thereof, a dose between about 100 mg of the compound of Formula I, or a pharmaceutically acceptable salt thereof, in simultaneous, separate or sequential combination with Temuterkib in the treatment of KRAS G12C-mutant advanced NSCLC.

Embodiment 167. A use of the compound of Formula I, or a pharmaceutically acceptable salt thereof, in the manufacture of a medicament for treatment of a KRAS G12C mutant cancer, administering to a patient in need thereof, a dose between about 50 mg of the compound of Formula I, or a pharmaceutically acceptable salt thereof, in simultaneous, separate or sequential combination with Temuterkib in the treatment of KRAS G12C-mutant advanced NSCLC.

Embodiment 168. A use of the compound of Formula I, or a pharmaceutically acceptable salt thereof, in the manufacture of a medicament for treatment of a KRAS G12C mutant cancer, administering to a patient in need thereof, a dose between about 150 mg of the compound of Formula I, or a pharmaceutically acceptable salt thereof, in simultaneous, separate or sequential combination with Temuterkib in the treatment of KRAS G12C-mutant advanced CRC.

Embodiment 169. A use of the compound of Formula I, or a pharmaceutically acceptable salt thereof, in the manufacture of a medicament for treatment of a KRAS G12C mutant cancer, administering to a patient in need thereof, a dose between about 100 mg of the compound of Formula I, or a pharmaceutically acceptable salt thereof, in simultaneous, separate or sequential combination with Temuterkib in the treatment of KRAS G12C-mutant advanced CRC.

Embodiment 170. A use of the compound of Formula I, or a pharmaceutically acceptable salt thereof, in the manufacture of a medicament for treatment of a KRAS G12C mutant cancer, administering to a patient in need thereof, a dose between about 50 mg of the compound of Formula I, or a pharmaceutically acceptable salt thereof, in simultaneous, separate or sequential combination with Temuterkib in the treatment of KRAS G12C-mutant advanced CRC.

Embodiment 171. A use of the compound of Formula I, or a pharmaceutically acceptable salt thereof, in the manufacture of a medicament for treatment of a KRAS G12C mutant cancer, administering to a patient in need thereof, a dose of about 150 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 172. A use of the compound of Formula I, or a pharmaceutically acceptable salt thereof, in the manufacture of a medicament for treatment of a KRAS G12C mutant cancer, administering to a patient in need thereof, a dose of about 100 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

Embodiment 173. A use of the compound of Formula I, or a pharmaceutically acceptable salt thereof, in the manufacture of a medicament for treatment of a KRAS G12C mutant cancer, administering to a patient in need thereof, a dose of about 50 mg of the compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid:2-methylpropan-2-amine (1:1) salt in the treatment of KRAS G12C-mutant advanced NSCLC.

1. A method of treating a KRAS G12C mutant cancer comprising:
administering to a patient in need of such treatment, a dose between about 50 mg and about 200 mg of a compound of Formula I: 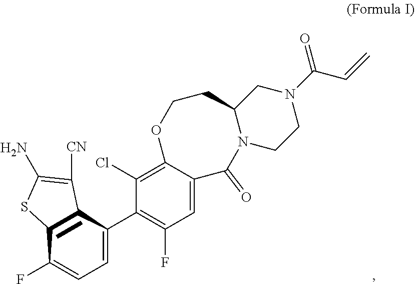
or a pharmaceutically acceptable salt thereof.

2. The method of claim 1, wherein administering the compound.

3. The method of claim 1, wherein the dose administered is a maximum daily dose selected from the group consisting of about 50 mg, about 100 mg, about 150 mg, about 200 mg, about 300 mg, about 400 mg, about 500 mg, about 600 mg, about 700 mg, and about 800 mg.

4. The method of claim 3, wherein the maximum daily dose is about 50 mg.

5. The method of claim 3, wherein the maximum daily dose is about 100 mg.

6. The method of claim 3, wherein the maximum daily dose is about 200 mg.

7. The method of claim 3, wherein the maximum daily dose is about 300 mg.

8. The method of claim 3, wherein the maximum daily dose is about 400 mg.

9. The method of claim 1, wherein the KRAS G12C mutant cancer is selected from the group consisting of lung cancer, colorectal cancer, pancreatic cancer, bladder cancer, cervical cancer, endometrial cancer, ovarian cancer, cholangiocarcinoma, and esophageal cancer.

10. The method of claim 9, wherein the KRAS G12C mutant cancer is non-small cell lung cancer, pancreatic cancer, or colorectal cancer.

11. The method of claim 10, wherein the KRAS G12C mutant cancer is non-small cell lung cancer or colorectal cancer.

12. The method of claim 9, wherein the KRAS G12C mutant cancer is non-small cell lung cancer.

13. The method of claim 9, wherein the KRAS G12C mutant cancer is pancreatic cancer.

14. The method of claim 9, wherein the KRAS G12C mutant cancer is colorectal cancer.

15. The method of claim 1, wherein the dose is administered to the patient at least once a day.

16. The method of claim 15, wherein the dose is selected from the group consisting of about 50 mg, about 100 mg, about 150 mg, and about 200 mg.

17. The method of claim 1, wherein the dose is in a capsule.

18. The method of claim 17 wherein the capsule contains about 25 mg or about 50 mg of the compound of Formula I, or a pharmaceutically acceptable salt thereof.

19. The method of claim 1, wherein the dose is administered to the patient at least twice a day.

20. The method of claim 1, wherein the dose is administered as a dose of about 50 mg to the patient at least twice a day.

21. The method of claim 1, wherein the dose is administered as a dose of about 100 mg to the patient at least twice a day.

22. The method of claim 1, wherein the dose is administered as a dose of about 150 mg to the patient at least twice a day.

23. The method of claim 1, wherein the dose is administered as a dose of about 200 mg to the patient at least twice a day.

24. The method of claim 1, comprising:
monitoring the patient for a dose limiting toxicity (DLT); and
administering a second dose of the compound of Formula I, or a pharmaceutically acceptable salt thereof, if the patient exhibits the DLT, wherein the second dose is reduced as compared to a first dose.

25. The method of claim 24, wherein the first dose is selected from the group consisting of about 50 mg, about 100 mg, about 150 mg, and about 200 mg.

26. The method of claim 24, wherein the second dose is selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg and about 150 mg.

27. The method of claim 24, wherein the second dose is reduced by 50 mg as compared to the first dose.

28. The method of claim 26, wherein the second dose is selected from the group consisting of about 50 mg, about 100 mg, and about 150 mg.

29. The method of claim 24, wherein the DLT includes a treatment-emergent adverse event and a clinically significant determination.

30. The method of claim 24, wherein the DLT occurs during the first 21 days of administering the first dose.

31. The method of claim 24, further comprising the steps of:
(a) monitoring the patient for a DLT; and
(b) administering a third dose of the compound of Formula I, or a pharmaceutically acceptable salt thereof, if the patient exhibits a DLT, wherein the third dose is reduced as compared to the second dose.

32. The method of claim 31, wherein the DLT includes a treatment-emergent adverse event and a clinically significant determination.

33. The method of claim 31, wherein the DLT occurs during the first 21 days of administering the second dose.

34. The method of claim 32, wherein the treatment-emergent adverse event is grade 3 or higher.

35. The method of claim 32, wherein the treatment-emergent adverse event is grade 4.

36. The method of claim 31, wherein the third dose is reduced by 50 mg as compared to the second dose.

37. A method of treating a KRAS G12C mutant cancer comprising:
administering to a patient in need thereof, a dose between about 50 mg and about 200 mg of the compound of Formula I, or a pharmaceutically acceptable salt thereof, in simultaneous, separate or sequential combination with a second therapeutic agent.

38. The method of claim 37, wherein the second therapeutic agent is selected from one or more of the group consisting of: a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

39. The method of claim 38, wherein the PD-1 or PD-L1 inhibitor is pembrolizumab.

40. The method of claim 38, wherein the platinum agent is cisplatin.

41. The method of claim 38, wherein the platinum agent is carboplatin.

42. The method of claim 38, wherein the antifolate is pemetrexed.

43. A method of treating a KRAS G12C mutant cancer, comprising:
administering to a patient in need thereof, a dose between about 50 mg and about 200 mg of the compound of Formula I, or a pharmaceutically acceptable salt thereof, in simultaneous, separate or sequential combination with pembrolizumab in the treatment of KRAS G12C-mutant advanced NSCLC.

44. A method of treating a KRAS G12C mutant cancer comprising:
administering to a patient in need of thereof, a dose between about 50 mg and about 200 mg of the compound of Formula I: 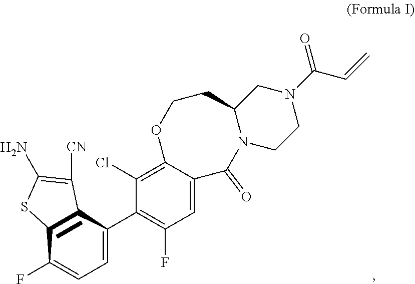
or a pharmaceutically acceptable salt thereof, wherein the KRAS G12C mutant cancer is KRAS G12C-mutant pancreatic cancer.
